SEQUENCE LISTING (90 sequences)

SEQ ID NO: 1 - RNA, 28 nt, Homo sapiens
gagaagcagaugaauaugaguccaccuc

SEQ ID NO: 2 - RNA, 28 nt, Homo sapiens
gaacgagguagagaagcagaugaauaug

SEQ ID NO: 3 - RNA, 28 nt, Homo sapiens
gaagcugagaagcuaaaggagcuacaga

SEQ ID NO: 4 - RNA, 28 nt, Homo sapiens
gggcuagagcgacaucaugguauucccc

SEQ ID NO: 5 - RNA, 28 nt, Homo sapiens
cugugugacaaauuuaguggccauccca

SEQ ID NO: 6 - RNA, 28 nt, Homo sapiens
gacuauggugcaacagcagaagagcugg

SEQ ID NO: 7 - RNA, 28 nt, Homo sapiens
cgagguagagaagcagaugaauaugagu

SEQ ID NO: 8 - RNA, 28 nt, Homo sapiens
cagugguuuuaacagcaggccccggggu

SEQ ID NO: 9 - RNA, 28 nt, Homo sapiens
agagcgacaucaugguauuccccuuacu

SEQ ID NO: 10 - RNA, 28 nt, Homo sapiens
gguagagaagcagaugaauaugagucca

SEQ ID NO: 11 - RNA, 28 nt, Homo sapiens
auugaggagaagauggaggcugaugccc

SEQ ID NO: 12 - RNA, 28 nt, Homo sapiens
ggaggaagaagcugagaagcuaaaggag

SEQ ID NO: 13 - RNA, 28 nt, Homo sapiens
aacgagguagagaagcagaugaauauga

SEQ ID NO: 14 - RNA, 20 nt, Artificial sequence
  Effector sequence for shmiR2
agcagaugaauaugagucca

SEQ ID NO: 15 - RNA, 21 nt, Artificial sequence
  Effector complement sequence for shmiR2
uggacucauauucaucugcuu

SEQ ID NO: 16 - RNA, 20 nt, Artificial sequence
  Effector sequence for shmiR3
gagguagagaagcagaugaa

SEQ ID NO: 17 - RNA, 21 nt, Artificial sequence
  Effector complement sequence for shmiR3
uucaucugcuucucuaccucg

SEQ ID NO: 18 - RNA, 20 nt, Artificial sequence
  Effector sequence for shmiR4
cugagaagcuaaaggagcua

SEQ ID NO: 19 - RNA, 21 nt, Artificial sequence
  Effector complement sequence for shmiR4
uagcuccuuuagcuucucagc

SEQ ID NO: 20 - RNA, 20 nt, Artificial sequence
  Effector sequence for shmiR5
uagagcgacaucaugguauu

SEQ ID NO: 21 - RNA, 21 nt, Artificial sequence
  Effector complement sequence for shmiR5
aauaccaugaugucgcucuag

SEQ ID NO: 22 - RNA, 20 nt, Artificial sequence
  Effector sequence for shmiR6
gugacaaauuuaguggccau

SEQ ID NO: 23 - RNA, 21 nt, Artificial sequence
  Effector complement sequence for shmiR6
auggccacuaaauuugucaca

SEQ ID NO: 24 - RNA, 20 nt, Artificial sequence
  Effector sequence for shmiR7
auggugcaacagcagaagag

SEQ ID NO: 25 - RNA, 21 nt, Artificial sequence
  Effector complement sequence for shmiR7
cucuucugcuguugcaccaua

SEQ ID NO: 26 - RNA, 20 nt, Artificial sequence
  Effector sequence for shmiR9
guagagaagcagaugaauau

SEQ ID NO: 27 - RNA, 21 nt, Artificial sequence
  Effector complement sequence for shmiR9
auauucaucugcuucucuacc

SEQ ID NO: 28 - RNA, 20 nt, Artificial sequence
  Effector sequence for shmiR11
gguuuuaacagcaggccccg

SEQ ID NO: 29 - RNA, 21 nt, Artificial sequence
  Effector complement sequence for shmiR11
cggggccugcuguuaaaacca

SEQ ID NO: 30 - RNA, 20 nt, Artificial sequence
  Effector sequence for shmiR13
cgacaucaugguauuccccu

SEQ ID NO: 31 - RNA, 21 nt, Artificial sequence
  Effector complement sequence for shmiR13
aggggaauaccaugaugucgc

SEQ ID NO: 32 - RNA, 20 nt, Artificial sequence
  Effector sequence for shmiR14
gagaagcagaugaauaugag

SEQ ID NO: 33 - RNA, 21 nt, Artificial sequence
  Effector complement sequence for shmiR14
cucauauucaucugcuucucu

SEQ ID NO: 34 - RNA, 20 nt, Artificial sequence
  Effector sequence for shmiR15
aggagaagauggaggcugau

SEQ ID NO: 35 - RNA, 21 nt, Artificial sequence
  Effector complement sequence for shmiR15
aucagccuccaucuucuccuc

SEQ ID NO: 36 - RNA, 20 nt, Artificial sequence
  Effector sequence for shmiR16
gaagaagcugagaagcuaaa

SEQ ID NO: 37 - RNA, 21 nt, Artificial sequence
  Effector complement sequence for shmiR16
uuuagcuucucagcuucuucc

SEQ ID NO: 38 - RNA, 20 nt, Artificial sequence
  Effector sequence for shmiR17
agguagagaagcagaugaau

SEQ ID NO: 39 - RNA, 21 nt, Artificial sequence
  Effector complement sequence for shmiR17
auucaucugcuucucuaccuc

SEQ ID NO: 40 - RNA, 18 nt, Artificial sequence
  Stem loop
acugugaagcagaugggu

SEQ ID NO: 41 - RNA, 26 nt, Artificial sequence
  5′ flanking sequence of the pri-miRNA backbone
  misc_feature (26)..(26): n is u or a
gguauauugcuguugacagugagcgn

SEQ ID NO: 42 - RNA, 22 nt, Artificial sequence
  3′ flanking sequence of the pri-miRNA backbone
cgccuacugccucggacuucaa

SEQ ID NO: 43 - RNA, 107 nt, Artificial sequence
  RNA sequence encoding shmiR2
gguauauugcuguugacagugagcguagcagaugaauaugaguccaacugugaagcagauggguuggacucauauucaucugcuucgccuacugccucggacuucaa

SEQ ID NO: 44 - RNA, 107 nt, Artificial sequence
  RNA sequence encoding shmiR3
gguauauugcuguugacagugagcgagagguagagaagcagaugaaacugugaagcagauggguuucaucugcuucucuaccucgcgccuacugccucggacuucaa

SEQ ID NO: 45 - RNA, 107 nt, Artificial sequence
  RNA sequence encoding shmiR4
gguauauugcuguugacagugagcgacugagaagcuaaaggagcuaacugugaagcagauggguuagcuccuuuagcuucucagccgccuacugccucggacuucaa

SEQ ID NO: 46 - RNA, 107 nt, Artificial sequence
  RNA sequence encoding shmiR5
gguauauugcuguugacagugagcgauagagcgacaucaugguauuacugugaagcagauggguaauaccaugaugucgcucuagcgccuacugccucggacuucaa

SEQ ID NO: 47 - RNA, 107 nt, Artificial sequence
  RNA sequence encoding shmiR6
gguauauugcuguugacagugagcgagugacaaauuuaguggccauacugugaagcagauggguauggccacuaaauuugucacacgccuacugccucggacuucaa

SEQ ID NO: 48 - RNA, 107 nt, Artificial sequence
  RNA sequence encoding shmiR7
gguauauugcuguugacagugagcgaauggugcaacagcagaagagacugugaagcagaugggucucuucugcuguugcaccauacgccuacugccucggacuucaa

SEQ ID NO: 49 - RNA, 107 nt, Artificial sequence
  RNA sequence encoding shmiR9
gguauauugcuguugacagugagcgaguagagaagcagaugaauauacugugaagcagauggguauauucaucugcuucucuacccgccuacugccucggacuucaa

SEQ ID NO: 50 - RNA, 107 nt, Artificial sequence
  RNA sequence encoding shmiR11
gguauauugcuguugacagugagcgagguuuuaacagcaggccccgacugugaagcagaugggucggggccugcuguuaaaaccacgccuacugccucggacuucaa

SEQ ID NO: 51 - RNA, 107 nt, Artificial sequence
  RNA sequence encoding shmiR13
gguauauugcuguugacagugagcgacgacaucaugguauuccccuacugugaagcagauggguaggggaauaccaugaugucgccgccuacugccucggacuucaa

SEQ ID NO: 52 - RNA, 107 nt, Artificial sequence
  RNA sequence encoding shmiR14
gguauauugcuguugacagugagcgugagaagcagaugaauaugagacugugaagcagaugggucucauauucaucugcuucucucgccuacugccucggacuucaa

SEQ ID NO: 53 - RNA, 107 nt, Artificial sequence
  RNA sequence encoding shmiR15
gguauauugcuguugacagugagcgaaggagaagauggaggcugauacugugaagcagauggguaucagccuccaucuucuccuccgccuacugccucggacuucaa

SEQ ID NO: 54 - RNA, 107 nt, Artificial sequence
  RNA sequence encoding shmiR16
gguauauugcuguugacagugagcgagaagaagcugagaagcuaaaacugugaagcagauggguuuuagcuucucagcuucuucccgccuacugccucggacuucaa

SEQ ID NO: 55 - RNA, 107 nt, Artificial sequence
  RNA sequence encoding shmiR17
gguauauugcuguugacagugagcgaagguagagaagcagaugaauacugugaagcagauggguauucaucugcuucucuaccuccgccuacugccucggacuucaa

SEQ ID NO: 56 - DNA, 107 bp, Artificial sequence
  DNA sequence encoding shmiR2
ggtatattgctgttgacagtgagcgtagcagatgaatatgagtccaactgtgaagcagatgggttggactcatattcatctgcttcgcctactgcctcggacttcaa

SEQ ID NO: 57 - DNA, 107 bp, Artificial sequence
  DNA sequence encoding shmiR3
ggtatattgctgttgacagtgagcgagaggtagagaagcagatgaaactgtgaagcagatgggtttcatctgcttctctacctcgcgcctactgcctcggacttcaa

SEQ ID NO: 58 - DNA, 107 bp, Artificial sequence
  DNA sequence encoding shmiR4
ggtatattgctgttgacagtgagcgactgagaagctaaaggagctaactgtgaagcagatgggttagctcctttagcttctcagccgcctactgcctcggacttcaa

SEQ ID NO: 59 - DNA, 107 bp, Artificial sequence
  DNA sequence encoding shmiR5
ggtatattgctgttgacagtgagcgatagagcgacatcatggtattactgtgaagcagatgggtaataccatgatgtcgctctagcgcctactgcctcggacttcaa

SEQ ID NO: 60 - DNA, 107 bp, Artificial sequence
  DNA sequence encoding shmiR6
ggtatattgctgttgacagtgagcgagtgacaaatttagtggccatactgtgaagcagatgggtatggccactaaatttgtcacacgcctactgcctcggacttcaa

SEQ ID NO: 61 - DNA, 107 bp, Artificial sequence
  DNA sequence encoding shmiR7
ggtatattgctgttgacagtgagcgaatggtgcaacagcagaagagactgtgaagcagatgggtctcttctgctgttgcaccatacgcctactgcctcggacttcaa

SEQ ID NO: 62 - DNA, 107 bp, Artificial sequence
  DNA sequence encoding shmiR9
ggtatattgctgttgacagtgagcgagtagagaagcagatgaatatactgtgaagcagatgggtatattcatctgcttctctacccgcctactgcctcggacttcaa

SEQ ID NO: 63 - DNA, 107 bp, Artificial sequence
  DNA sequence encoding shmiR11
ggtatattgctgttgacagtgagcgaggttttaacagcaggccccgactgtgaagcagatgggtcggggcctgctgttaaaaccacgcctactgcctcggacttcaa

SEQ ID NO: 64 - DNA, 107 bp, Artificial sequence
  DNA sequence encoding shmiR13
ggtatattgctgttgacagtgagcgacgacatcatggtattcccctactgtgaagcagatgggtaggggaataccatgatgtcgccgcctactgcctcggacttcaa

SEQ ID NO: 65 - DNA, 107 bp, Artificial sequence
  DNA sequence encoding shmiR14
ggtatattgctgttgacagtgagcgtgagaagcagatgaatatgagactgtgaagcagatgggtctcatattcatctgcttctctcgcctactgcctcggacttcaa

SEQ ID NO: 66 - DNA, 107 bp, Artificial sequence
  DNA sequence encoding shmiR15
ggtatattgctgttgacagtgagcgaaggagaagatggaggctgatactgtgaagcagatgggtatcagcctccatcttctcctccgcctactgcctcggacttcaa

SEQ ID NO: 67 - DNA, 107 bp, Artificial sequence
  DNA sequence encoding shmiR16
ggtatattgctgttgacagtgagcgagaagaagctgagaagctaaaactgtgaagcagatgggttttagcttctcagcttcttcccgcctactgcctcggacttcaa

SEQ ID NO: 68 - DNA, 107 bp, Artificial sequence
  DNA sequence encoding shmiR17
ggtatattgctgttgacagtgagcgaaggtagagaagcagatgaatactgtgaagcagatgggtattcatctgcttctctacctccgcctactgcctcggacttcaa

SEQ ID NO: 69 - DNA, 2532 bp, Artificial sequence
  Double expression construct version 1 coding for shmiR3, shmiR14 and codon optimized PABPN1
cgatcgcgcgcagatctgtcatgatgatcctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgatcattgaagttactattccgaagttcctattctctagaattcgccaccacgcgtggtatattgctgttgacagtgagcgagaggtagagaagcagatgaaactgtgaagcagatgggtttcatctgcttctctacctcgcgcctactgcctcggacttcaaatcatctactccatggccctctgcgtttgctgaagacagaaccgcaaagcaggacccgacaggattctccccgcctcttcagagactatgtttacaagatatcggtatattgctgttgacagtgagcgtgagaagcagatgaatatgagactgtgaagcagatgggtctcatattcatctgcttctctcgcctactgcctcggacttcaagtcgacgctagcaataaaggatcctttattttcattggatccgtgtgttggttttttgtgtgcggttaattaaggtacccgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatgccaccatggccgctgccgccgctgctgctgccgcagccggcgctgccggcggaagaggcagcggccctggcagacggcggcatctggtccctggcgccggaggggaggccggcgaaggcgcccctggcggagccggcgactacggcaacggcctggaaagcgaggaactggaacccgaggaactgctgctggaacctgagcccgagccagagcccgaggaagagccccctaggccaagagccccccctggcgccccaggaccaggaccaggctctggggcaccaggctctcaggaagaggaagaagagcccggcctcgtcgagggagacccaggcgatggcgctatcgaagatcccgagctggaagccatcaaggccagagtgcgggagatggaagaggaggccgaaaaattgaaagagctgcagaacgaagtcgaaaaacaaatgaacatgtccccccctcctggaaatgctggccctgtgatcatgagcatcgaggaaaagatggaagccgacgcccggtctatctacgtgggcaacgtggactacggcgccaccgccgaagaactggaagcccactttcacggctgtggcagcgtgaaccgggtgaccatcctgtgcgacaagttcagcggccaccccaagggcttcgcctacatcgagttcagcgacaaagaaagcgtgcggacctctctggctctcgacgagtctctgttcaggggaaggcagatcaaggtcatccccaagcggaccaacaggcccggcatcagcaccaccgacagaggcttccctagggctaggtacagagcccggaccaccaactacaacagcagcagaagccggttctacagcggcttcaattctcggcctagaggcagagtgtaccggggcagggccagggccacctcctggtacagcccctacgaacagaagctgatcagcgaggaagatctgtgatgagatatctgatgacatatgacgcgtttaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 70 - DNA, 2532 bp, Artificial sequence
  Double expression construct version 1 coding for shmiR17, shmiR13 and codon optimized PABPN1
cgatcgcgcgcagatctgtcatgatgatcctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgatcattgaagttactattccgaagttcctattctctagaattcgccaccacgcgtggtatattgctgttgacagtgagcgaaggtagagaagcagatgaatactgtgaagcagatgggtattcatctgcttctctacctccgcctactgcctcggacttcaaatcatctactccatggccctctgcgtttgctgaagacagaaccgcaaagcaggacccgacaggattctccccgcctcttcagagactatgtttacaagatatcggtatattgctgttgacagtgagcgacgacatcatggtattcccctactgtgaagcagatgggtaggggaataccatgatgtcgccgcctactgcctcggacttcaagtcgacgctagcaataaaggatcctttattttcattggatccgtgtgttggttttttgtgtgcggttaattaaggtacccgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatgccaccatggccgctgccgccgctgctgctgccgcagccggcgctgccggcggaagaggcagcggccctggcagacggcggcatctggtccctggcgccggaggggaggccggcgaaggcgcccctggcggagccggcgactacggcaacggcctggaaagcgaggaactggaacccgaggaactgctgctggaacctgagcccgagccagagcccgaggaagagccccctaggccaagagccccccctggcgccccaggaccaggaccaggctctggggcaccaggctctcaggaagaggaagaagagcccggcctcgtcgagggagacccaggcgatggcgctatcgaagatcccgagctggaagccatcaaggccagagtgcgggagatggaagaggaggccgaaaaattgaaagagctgcagaacgaagtcgaaaaacaaatgaacatgtccccccctcctggaaatgctggccctgtgatcatgagcatcgaggaaaagatggaagccgacgcccggtctatctacgtgggcaacgtggactacggcgccaccgccgaagaactggaagcccactttcacggctgtggcagcgtgaaccgggtgaccatcctgtgcgacaagttcagcggccaccccaagggcttcgcctacatcgagttcagcgacaaagaaagcgtgcggacctctctggctctcgacgagtctctgttcaggggaaggcagatcaaggtcatccccaagcggaccaacaggcccggcatcagcaccaccgacagaggcttccctagggctaggtacagagcccggaccaccaactacaacagcagcagaagccggttctacagcggcttcaattctcggcctagaggcagagtgtaccggggcagggccagggccacctcctggtacagcccctacgaacagaagctgatcagcgaggaagatctgtgatgagatatctgatgacatatgacgcgtttaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 71 - DNA, 1943 bp, Artificial sequence
  Double expression construct version 2 coding for shmiR3, shmiR14 and codon optimized PABPN1
cgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatgccaccatggccgctgccgccgctgctgctgccgcagccggcgctgccggcggaagaggcagcggccctggcagacggcggcatctggtccctggcgccggaggggaggccggcgaaggcgcccctggcggagccggcgactacggcaacggcctggaaagcgaggaactggaacccgaggaactgctgctggaacctgagcccgagccagagcccgaggaagagccccctaggccaagagccccccctggcgccccaggaccaggaccaggctctggggcaccaggctctcaggaagaggaagaagagcccggcctcgtcgagggagacccaggcgatggcgctatcgaagatcccgagctggaagccatcaaggccagagtgcgggagatggaagaggaggccgaaaaattgaaagagctgcagaacgaagtcgaaaaacaaatgaacatgtccccccctcctggaaatgctggccctgtgatcatgagcatcgaggaaaagatggaagccgacgcccggtctatctacgtgggcaacgtggactacggcgccaccgccgaagaactggaagcccactttcacggctgtggcagcgtgaaccgggtgaccatcctgtgcgacaagttcagcggccaccccaagggcttcgcctacatcgagttcagcgacaaagaaagcgtgcggacctctctggctctcgacgagtctctgttcaggggaaggcagatcaaggtcatccccaagcggaccaacaggcccggcatcagcaccaccgacagaggcttccctagggctaggtacagagcccggaccaccaactacaacagcagcagaagccggttctacagcggcttcaattctcggcctagaggcagagtgtaccggggcagggccagggccacctcctggtacagcccctactgatgacatatgacgcgtggtatattgctgttgacagtgagcgagaggtagagaagcagatgaaactgtgaagcagatgggtttcatctgcttctctacctcgcgcctactgcctcggacttcaaatcatctactccatggccctctgcgtttgctgaagacagaaccgcaaagcaggacccgacaggattctccccgcctcttcagagactatgtttacaagatatcggtatattgctgttgacagtgagcgtgagaagcagatgaatatgagactgtgaagcagatgggtctcatattcatctgcttctctcgcctactgcctcggacttcaagtcgacgctagcaataaaggatcctttattttcattggatccgtgtgttggttttttgtgtgcggttaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 72 - DNA, 1943 bp, Artificial sequence
  Double expression construct version 2 coding for shmiR17, shmiR13 and codon optimized PABPN1
cgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatgccaccatggccgctgccgccgctgctgctgccgcagccggcgctgccggcggaagaggcagcggccctggcagacggcggcatctggtccctggcgccggaggggaggccggcgaaggcgcccctggcggagccggcgactacggcaacggcctggaaagcgaggaactggaacccgaggaactgctgctggaacctgagcccgagccagagcccgaggaagagccccctaggccaagagccccccctggcgccccaggaccaggaccaggctctggggcaccaggctctcaggaagaggaagaagagcccggcctcgtcgagggagacccaggcgatggcgctatcgaagatcccgagctggaagccatcaaggccagagtgcgggagatggaagaggaggccgaaaaattgaaagagctgcagaacgaagtcgaaaaacaaatgaacatgtccccccctcctggaaatgctggccctgtgatcatgagcatcgaggaaaagatggaagccgacgcccggtctatctacgtgggcaacgtggactacggcgccaccgccgaagaactggaagcccactttcacggctgtggcagcgtgaaccgggtgaccatcctgtgcgacaagttcagcggccaccccaagggcttcgcctacatcgagttcagcgacaaagaaagcgtgcggacctctctggctctcgacgagtctctgttcaggggaaggcagatcaaggtcatccccaagcggaccaacaggcccggcatcagcaccaccgacagaggcttccctagggctaggtacagagcccggaccaccaactacaacagcagcagaagccggttctacagcggcttcaattctcggcctagaggcagagtgtaccggggcagggccagggccacctcctggtacagcccctactgatgacatatgacgcgtggtatattgctgttgacagtgagcgaaggtagagaagcagatgaatactgtgaagcagatgggtattcatctgcttctctacctccgcctactgcctcggacttcaaatcatctactccatggccctctgcgtttgctgaagacagaaccgcaaagcaggacccgacaggattctccccgcctcttcagagactatgtttacaagatatcggtatattgctgttgacagtgagcgacgacatcatggtattcccctactgtgaagcagatgggtaggggaataccatgatgtcgccgcctactgcctcggacttcaagtcgacgctagcaataaaggatcctttattttcattggatccgtgtgttggttttttgtgtgcggttaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 73 - DNA, 921 bp, Artificial sequence
  Human codon-optimized PABPN1 cDNA sequence
atggccgctgccgccgctgctgctgccgcagccggcgctgccggcggaagaggcagcggccctggcagacggcggcatctggtccctggcgccggaggggaggccggcgaaggcgcccctggcggagccggcgactacggcaacggcctggaaagcgaggaactggaacccgaggaactgctgctggaacctgagcccgagccagagcccgaggaagagccccctaggccaagagccccccctggcgccccaggaccaggaccaggctctggggcaccaggctctcaggaagaggaagaagagcccggcctcgtcgagggagacccaggcgatggcgctatcgaagatcccgagctggaagccatcaaggccagagtgcgggagatggaagaggaggccgaaaaattgaaagagctgcagaacgaagtcgaaaaacaaatgaacatgtccccccctcctggaaatgctggccctgtgatcatgagcatcgaggaaaagatggaagccgacgcccggtctatctacgtgggcaacgtggactacggcgccaccgccgaagaactggaagcccactttcacggctgtggcagcgtgaaccgggtgaccatcctgtgcgacaagttcagcggccaccccaagggcttcgcctacatcgagttcagcgacaaagaaagcgtgcggacctctctggctctcgacgagtctctgttcaggggaaggcagatcaaggtcatccccaagcggaccaacaggcccggcatcagcaccaccgacagaggcttccctagggctaggtacagagcccggaccaccaactacaacagcagcagaagccggttctacagcggcttcaattctcggcctagaggcagagtgtaccggggcagggccagggccacctcctggtacagcccctactga

SEQ ID NO: 74 - protein, 306 aa, Artificial sequence
  Human wildtype PABPN1 amino acid sequence
MAAAAAAAAAAGAAGGRGSGPGRRRHLVPGAGGEAGEGAPGGAGDYGNGLESEELEPEELLLEPEPEPEPEEEPPRPRAPPGAPGPGPGSGAPGSQEEEEEPGLVEGDPGDGAIEDPELEAIKARVREMEEEAEKLKELQNEVEKQMNMSPPPGNAGPVIMSIEEKMEADARSIYVGNVDYGATAEELEAHFHGCGSVNRVTILCDKFSGHPKGFAYIEFSDKESVRTSLALDESLFRGRQIKVIPKRTNRPGISTTDRGFPRARYRARTTNYNSSRSRFYSGFNSRPRGRVYRGRARATSWYSPY

SEQ ID NO: 75 - protein, 314 aa, Artificial sequence
  Human wildtype PABPN1 amino acid sequence (with FLAG tag)
MAAAAAAAAAAGAAGGRGSGPGRRRHLVPGAGGEAGEGAPGGAGDYGNGLESEELEPEELLLEPEPEPEPEEEPPRPRAPPGAPGPGPGSGAPGSQEEEEEPGLVEGDPGDGAIEDPELEAIKARVREMEEEAEKLKELQNEVEKQMNMSPPPGNAGPVIMSIEEKMEADARSIYVGNVDYGATAEELEAHFHGCGSVNRVTILCDKFSGHPKGFAYIEFSDKESVRTSLALDESLFRGRQIKVIPKRTNRPGISTTDRGFPRARYRARTTNYNSSRSRFYSGFNSRPRGRVYRGRARATSWYSPYDYKDDDDK

SEQ ID NO: 76 - protein, 314 aa, Artificial sequence; identical to SEQ ID NO: 75
  Human codon-optimized PABPN1 amino acid sequence (with FLAG-tag)

SEQ ID NO: 77 - DNA, 20 bp, Artificial sequence
  wtPABPN1-Fwd primer
atggtgcaacagcagaagag

SEQ ID NO: 78 - DNA, 20 bp, Artificial sequence
  wtPABPN1-Rev primer
ctttgggatggccactaaat

SEQ ID NO: 79 - DNA, 23 bp, Artificial sequence
  wtPABPN1-Probe
cggttgactgaaccacagccatg

SEQ ID NO: 80 - DNA, 19 bp, Artificial sequence
  optPABPN1-For primer
accgacagaggcttcccta

SEQ ID NO: 81 - DNA, 21 bp, Artificial sequence
  optPABPN1-Rev primer
ttctgctgctgttgtagttgg

SEQ ID NO: 82 - DNA, 23 bp, Artificial sequence
  optPABPN1-Probe
tggtccgggctctgtacctagcc

SEQ ID NO: 83 - DNA, 21 bp, Artificial sequence
  shmiR3-Fwd primer
ttcatctgcttctctacctcg

SEQ ID NO: 84 - DNA, 21 bp, Artificial sequence
  shmiR13-Fwd primer
aggggaataccatgatgtcgc

SEQ ID NO: 85 - DNA, 21 bp, Artificial sequence
  shmiR14-Fwd primer
ctcatattcatctgcttctct

SEQ ID NO: 86 - DNA, 21 bp, Artificial sequence
  shmiR17-Fwd primer
attcatctgcttctctacctc

SEQ ID NO: 87 - RNA, 921 nt, homo sapien
auggcggcggcggcggcggcggcagcagcagcgggggcugcgggcggucggggcuccgggccggggcggcggcgccaucuugugcccggggccgguggggaggccggggagggggccccggggggcgcaggggacuacgggaacggccuggagucugaggaacuggagccugaggagcugcugcuggagcccgagccggagcccgagcccgaagaggagccgccccggccccgcgcccccccgggagcuccgggcccugggccugguucgggagcccccggcagccaagaggaggaggaggagccgggacuggucgagggugacccgggggacggcgccauugaggacccggagcuggaagcuaucaaagcucgagucagggagauggaggaagaagcugagaagcuaaaggagcuacagaacgagguagagaagcagaugaauaugaguccaccuccaggcaaugcuggcccggugaucauguccauugaggagaagauggaggcugaugcccguuccaucuauguuggcaauguggacuauggugcaacagcagaagagcuggaagcucacuuucauggcugugguucagucaaccguguuaccauacugugugacaaauuuaguggccaucccaaaggguuugcguauauagaguucucagacaaagagucagugaggacuuccuuggccuuagaugagucccuauuuagaggaaggcaaaucaaggugaucccaaaacgaaccaacagaccaggcaucagcacaacagaccgggguuuuccacgagcccgcuaccgcgcccggaccaccaacuacaacagcucccgcucucgauucuacagugguuuuaacagcaggccccggggucgcgucuacaggggccgggcuagagcgacaucaugguauuccccuuacuaa

SEQ ID NO: 88 - protein, 64 aa, Artificial Sequence
  Consensus AAV VP1 subsequence comprising PLA2 domain
  MISC_FEATURE (15)..(15): Xaa is Gly or Phe
  MISC_FEATURE (20)..(20): Xaa is Arg or Lys
  MISC_FEATURE (29)..(29): Xaa is Glu or Ala
  MISC_FEATURE (30)..(30): Xaa is Val or Ala
  MISC_FEATURE (32)..(32): Xaa is Arg or Leu
  MISC_FEATURE (36)..(36): Xaa is Ile or Lys
  MISC_FEATURE (37)..(37): Xaa is Ser or Ala
  MISC_FEATURE (39)..(39): Xaa is Asn or Asp
  MISC_FEATURE (51)..(51): Xaa is Lys or Arg
  MISC_FEATURE (61)..(61): Xaa is Glu or Gln
  MISC_FEATURE (62)..(62): Xaa is Lys or Arg
SRGLVLPGYNYLGPXNGLDXGEPVNEADXXAXEHDXXYXRQLDSGDNPYLXYNHADAEFQXXLK

SEQ ID NO: 89 - protein, 64 aa, Artificial Sequence
  Modified VP1 PLA2 subsequence for AAV8
SRGLVLPGYKYLGPFNGLDKGEPVNEADAAALEHDKAYDRQLDSGDNPYLRYNHADAEFQERLK

SEQ ID NO: 90 - protein, 64 aa, Artificial Sequence
  Modified VP1 PLA2 subsequence for AAV9
SRGLVLPGYKYLGPGNGLDKGEPVNEADAAALEHDKAYDRQLDSGDNPYLKYNHADAEFQERLK

(19) United States
(12) Patent Application Publication
(10) Pub. No.: US 2022/0106594 A1
(43) Pub. Date: Apr. 7, 2022

(54) Methods for Treating Oculopharyngeal Muscular Dystrophy (OPMD)

(71) Applicant: Benitec Biopharma Limited, Hayward, CA (US)

(72) Inventors: Vanessa Strings-Ufombah, Hayward, CA (US); David Suhy, Hayward, CA (US)

(21) Appl. No.: 17/285,569

(22) PCT Filed: Oct. 17, 2019
(86) PCT No.: PCT/AU2019/051134

§ 371 Date: Apr. 15, 2021

Related U.S. Application Data
(60) Provisional application No. 62/747,089, filed on Oct. 17, 2018.

(51) Int. Cl. C12N 15/113; A61K 31/7105; A61K 31/711; A61P 21/00

(52) U.S. Cl. CPC C12N 15/113; A61P 21/00; A61K 31/711; A61K 31/7105

(57) ABSTRACT

The present disclosure relates to methods of administering a gene therapy construct comprising RNA interference (RNAi) reagents, such as short hairpin microRNA (shmiR), in combination with PABPN1 replacement reagents, such as polynucleotides which encode functional PABPN1 protein which are not targeted by the RNAi reagents, for treatment of oculopharyngeal muscular dystrophy (OPMD) in individuals suffering from OPMD or which are predisposed thereto. In certain aspects the method comprises direct injection to a subject's pharyngeal muscles.

### CROSS-REFERENCE TO RELATED APPLICATIONS

This application claims the right of priority to U.S. Provisional No. 62/747,089, filed 17 Oct. 2018, the complete contents of which is incorporated by reference herein in its entirety.

### TECHNICAL FIELD

The present disclosure relates to methods for treating oculopharyngeal muscular dystrophy (OPMD) in individuals suffering from OPMD or which are predisposed thereto.

### BACKGROUND

OPMD is an autosomal dominant inherited, slow progressing, late-onset degenerative muscle disorder. The disease is mainly characterised by progressive eyelid drooping (ptosis) and swallowing difficulties (dysphagia). The pharyngeal and cricopharyngeal muscles are specific targets in OPMD. Proximal limb weakness tends to follow at a later stage of disease progression. The mutation that causes the disease is an abnormal expansion of a (GCN)n trinucleotide repeat in the coding region of the poly(A) binding protein nuclear 1 (PABPN1) gene. This expansion leads to an expanded polyalanine tract at the N-terminal of the PABPN1 protein: 10 alanines are present in the normal protein, expanded to 11 to 18 alanines in the mutant form (expPABPN1). The main pathological hallmark of the disease is nuclear aggregates of expPABPN1. A misfolding of expanded PABPN1 results in the accumulation of insoluble polymeric fibrillar aggregates inside nuclei of affected cells. PABPN1 is an aggregation prone protein and mutant alanine-expanded PABPN1 in OPMD has a higher aggregation rate than that of the wild type normal protein. However, it is still unclear whether the nuclear aggregates in OPMD have a pathological function or a protective role as a consequence of a cellular defense mechanism.

No treatment, pharmacological or otherwise, is presently available for OPMD. Symptomatic surgical interventions can partly correct ptosis and improve swallowing in moderate to severely affected individuals. For example, the cricopharyngeal myotomy is at present the only possible treatment available to improve swallowing in these patients. However, this does not correct the progressive degradation of the pharyngeal musculature, which often leads to death following swallowing difficulties and chocking.

Accordingly, there remains a need for therapeutic agents to treat OPMD in patients suffering therefrom and/or who are predisposed thereto.

### SUMMARY

The present disclosure is based, in part, on the recognition by the inventors that no approved therapeutic agents currently exist for the treatment of OPMD. The present disclosure therefore provides methods for administering RNAi reagents targeting regions of the PABPN1 mRNA transcript which is causative of OPMD. Furthermore, the present disclosure provides methods for administering reagents for expression of wild-type human PABPN1 protein having a mRNA transcript which is not targeted by the RNAi reagents of the disclosure (hereinafter “PABPN1 replacement reagents”).

Certain aspects of the disclosure are directed to a method for treating a subject suffering from oculopharyngeal muscular dystrophy (OPMD) comprising administering to said subject a composition comprising:

(a) a nucleic acid comprising a DNA sequence which encodes a short hairpin micro-RNA (shmiR); and

(b) a PABPN1 construct comprising a DNA sequence encoding a functional PABPN1 protein having a mRNA transcript which is not targeted by the shmiR(s) encoded by the nucleic acid; wherein the composition is administered by direct injection to a pharyngeal muscle of the subject.

Certain aspects are directed to a method of inhibiting expression of a PABPN1 protein which is causative of oculopharyngeal muscular dystrophy (OPMD) in a subject, said method comprising administering to the subject a composition comprising:

(a) a ddRNAi construct comprising a nucleic acid comprising a DNA sequence which encodes a short hairpin micro-RNA (shmiR); and

(b) a PABPN1 construct comprising a DNA sequence encoding a functional PABPN1 protein having a mRNA transcript which is not targeted by the shmiR(s) encoded by the ddRNAi construct; wherein the composition is administered by direct injection to a pharyngeal muscle of the subject.

In one example, the subject has improved swallowing following administering the composition by direct injection to a pharyngeal muscle of the subject.

In one example, the composition comprises an expression vector comprising the ddRNAi construct, the PABPN1 construct, or a combination thereof.

In one example, the expression vector comprises, in a 5′ to 3′ direction, the ddRNAi construct and the PABPN1 construct.

In one example, the expression vector comprises, in a 5′ to 3′ direction, the PABPN1 construct and the ddRNAi construct.

In one example, the expression vector is a plasmid or minicircle.

In one example, the expression vector is a viral vector selected from the group consisting of an adeno-associated viral (AAV) vector, a retroviral vector, an adenoviral (AdV) vector and a lentiviral (LV) vector. For example, the expression vector may be an AAV vector e.g., an AAV from serotype AAV2, AAV8 or AAV9.

In one example, the nucleic acid, ddRNAi construct and/or PABPN1 construct is/are comprised with an expression construct and the expression construct comprises inverted terminal repeats (ITRs) from an AAV serotype.

In one example, the DNA sequence encoding the functional PABPN1 protein is codon optimised such that its mRNA transcript is not targeted by the shmiRs encoded by the nucleic acid or ddRNAi construct. For example, the DNA sequence encoding the functional PABPN1 protein may be the DNA sequence set forth in SEQ ID NO: 73.

In one example, the DNA sequence encoding the functional PABPN1 protein is operably-linked to a promoter comprised within the PABPN1 construct and positioned upstream of the DNA sequence encoding the functional PABPN1 protein. For example, the promoter comprised within the PABPN1 construct may be a muscle-specific promoter.

In one example, the nucleic acid comprises a DNA sequence which encodes a shmiR which targets an RNA transcript of human PABPN1, wherein the shmiR comprises:

an effector sequence of at least 17 nucleotides in length;

an effector complement sequence;

a stemloop sequence; and

primary micro RNA (pri-miRNA) backbone;

wherein the effector sequence is substantially complementary to a region of corresponding length in an RNA transcript of human PABPN1. For example, the effector sequence may be substantially complementary to a region of corresponding length within the sequence set forth in SEQ ID NO: 87 (i.e., the messenger RNA transcript encoding human PABPN1).

In some examples, the nucleic acid comprises a DNA sequence encoding a shmiR comprising:

an effector sequence of at least 17 nucleotides in length;

an effector complement sequence;

a stemloop sequence; and

pri-miRNA backbone;

wherein the effector sequence is substantially complementary to a region of corresponding length in an RNA transcript set forth in any one of SEQ ID NOs: 1-13.

Preferably, the effector sequence will be less than 30 nucleotides in length. For example, a suitable effector sequence may be in the range of 17-29 nucleotides in length. Preferably, the effector sequence will be 20 nucleotides in length. More preferably, the effector sequence will be 21 nucleotides in length and the effector complement sequence will be 20 nucleotides in length.

In certain examples, the shmiR encoded by the nucleic acid comprises an effector sequence which is substantially complementary to a region of corresponding length in an RNA transcript comprising or consisting of the sequence set forth in any one of SEQ ID NOs: 1-13 (i.e., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13). For example, the effector sequence may be substantially complementary to a region of corresponding length in an RNA transcript comprising or consisting of the sequence set forth in any one of SEQ ID NOs: 1-13 and contain 4 mismatch bases relative thereto. For example, the effector sequence may be substantially complementary to a region of corresponding length in an RNA transcript comprising or consisting of the sequence set forth in any one of SEQ ID NOs: 1-13 and contain 3 mismatch bases relative thereto. For example, the effector sequence may be substantially complementary to a region of corresponding length in an RNA transcript comprising or consisting of the sequence set forth in any one of SEQ ID NOs: 1-13 and contain 2 mismatch bases relative thereto. For example, the effector sequence may be substantially complementary to a region of corresponding length in an RNA transcript comprising or consisting of the sequence set forth in any one of SEQ ID NOs: 1-13 and contain 1 mismatch base relative thereto. For example, the effector sequence may be 100% complementary to a region of corresponding length in an RNA transcript comprising or consisting of the sequence set forth in any one of SEQ ID NOs: 1-13. Where mismatches are present, it is preferred that they are not located within the region corresponding to the seed region of the shmiR i.e., nucleotides 2-8 of the effector sequence.

Exemplary nucleic acids which may be useful in the method of the disclosure may comprise a DNA sequence encoding a shmiR having an effector/effector complement sequence combination as described in Table 2.

In one example, the shmiR encoded by the nucleic acid is a shmiR comprising an effector sequence which is substantially complementary to the sequence forth in SEQ ID NO: 14. For example, a shmiR comprising an effector sequence set forth in SEQ ID NO: 15 and an effector complement sequence set forth in SEQ ID NO: 14.

In one example, the shmiR encoded by the nucleic acid is a shmiR comprising an effector sequence which is substantially complementary to the sequence forth in SEQ ID NO: 16. For example, a shmiR comprising an effector sequence set forth in SEQ ID NO: 17 and an effector complement sequence set forth in SEQ ID NO: 16.

In one example, the shmiR encoded by the nucleic acid is a shmiR comprising an effector sequence which is substantially complementary to the sequence forth in SEQ ID NO: 18. For example, a shmiR comprising an effector sequence set forth in SEQ ID NO: 19 and an effector complement sequence set forth in SEQ ID NO: 18.

In one example, the shmiR encoded by the nucleic acid is a shmiR comprising an effector sequence which is substantially complementary to the sequence forth in SEQ ID NO: 20. For example, a shmiR comprising an effector sequence set forth in SEQ ID NO: 21 and an effector complement sequence set forth in SEQ ID NO: 20.

In one example, the shmiR encoded by the nucleic acid is a shmiR comprising an effector sequence which is substantially complementary to the sequence forth in SEQ ID NO: 22. For example, a shmiR comprising an effector sequence set forth in SEQ ID NO: 23 and an effector complement sequence set forth in SEQ ID NO: 22.

In one example, the shmiR encoded by the nucleic acid is a shmiR comprising an effector sequence which is substantially complementary to the sequence forth in SEQ ID NO: 24. For example, a shmiR comprising an effector sequence set forth in SEQ ID NO: 25 and an effector complement sequence set forth in SEQ ID NO: 24.

In one example, the shmiR encoded by the nucleic acid is a shmiR comprising an effector sequence which is substantially complementary to the sequence forth in SEQ ID NO: 26. For example, a shmiR comprising an effector sequence set forth in SEQ ID NO: 27 and an effector complement sequence set forth in SEQ ID NO: 26.

In one example, the shmiR encoded by the nucleic acid is a shmiR comprising an effector sequence which is substantially complementary to the sequence forth in SEQ ID NO: 28. For example, a shmiR comprising an effector sequence set forth in SEQ ID NO: 29 and an effector complement sequence set forth in SEQ ID NO: 28.

In one example, the shmiR encoded by the nucleic acid is a shmiR comprising an effector sequence which is substantially complementary to the sequence forth in SEQ ID NO: 30. For example, a shmiR comprising an effector sequence set forth in SEQ ID NO: 31 and an effector complement sequence set forth in SEQ ID NO: 30.

In one example, the shmiR encoded by the nucleic acid is a shmiR comprising an effector sequence which is substantially complementary to the sequence forth in SEQ ID NO: 32. For example, a shmiR comprising an effector sequence set forth in SEQ ID NO: 33 and an effector complement sequence set forth in SEQ ID NO: 32.

In one example, the shmiR encoded by the nucleic acid is a shmiR comprising an effector sequence which is substantially complementary to the sequence forth in SEQ ID NO: 34. For example, a shmiR comprising an effector sequence set forth in SEQ ID NO: 35 and an effector complement sequence set forth in SEQ ID NO: 34.

In one example, the shmiR encoded by the nucleic acid is a shmiR comprising an effector sequence which is substantially complementary to the sequence forth in SEQ ID NO: 36. For example, a shmiR comprising an effector sequence set forth in SEQ ID NO: 37 and an effector complement sequence set forth in SEQ ID NO: 36.

In one example, the shmiR encoded by the nucleic acid is a shmiR comprising an effector sequence which is substantially complementary to the sequence forth in SEQ ID NO: 38. For example, a shmiR comprising an effector sequence set forth in SEQ ID NO: 39 and an effector complement sequence set forth in SEQ ID NO: 38.

In one example, the shmiR comprises, in a 5′ to 3′ direction:

a 5′ flanking sequence of the pri-miRNA backbone;

the effector complement sequence;

the stemloop sequence;

the effector sequence; and

a 3′ flanking sequence of the pri-miRNA backbone.

In one example, the shmiR comprises, in a 5′ to 3′ direction:

a 5′ flanking sequence of the pri-miRNA backbone;

the effector sequence;

the stemloop sequence;

the effector complement sequence; and

a 3′ flanking sequence of the pri-miRNA backbone.

In one example, the stemloop sequence may be the sequence set forth in SEQ ID NO: 40.

In one example, the pri-miRNA backbone is a pri-miR-30a backbone. For example, the 5′ flanking sequence of the pri-miRNA backbone may be the sequence set forth in SEQ ID NO: 41 and the 3′ flanking sequence of the pri-miRNA backbone may be the set forth in SEQ ID NO: 42.

Exemplary nucleic acids which may be useful in the method of the disclosure may comprise a DNA sequence encoding a shmiR having a sequence as described in Table 3 and/or encoded by a sequence in Table 4. For example, the shmiR encoded by the nucleic acid of the disclosure may comprises a sequence set forth in any one of SEQ ID NOs: 43-55. The shmiR may be encoded by a DNA sequence set forth in any one of SEQ ID NOs: 56-68.

In some examples the method comprises administering at least two nucleic acids encoding shmiRs, or administering a ddRNAi construct comprising the at least two nucleic acids, wherein each shmiR comprises an effector sequence which is substantially complementary to a RNA transcript corresponding to a PABPN1 protein which is causative of OPMD, and wherein each shmiR comprises a different effector sequence.

The at least two nucleic acids may be administered separately or within a single ddRNAi construct. In one example, each of the at least two nucleic acids each encode a shmiR comprising an effector sequence which is substantially complementary to a region of corresponding length in an RNA transcript set forth in one of SEQ ID NOs: 1, 2, 4, 7, 9, 10 and 13. For example, the at least two nucleic acids may be selected from the group consisting of: a nucleic acid comprising or consisting of a DNA sequence encoding a shmiR comprising an effector sequence set forth in SEQ ID NO: 15 and an effector complement sequence set forth in SEQ ID NO: 14 (shmiR2); a nucleic acid comprising or consisting of a DNA sequence encoding a shmiR comprising an effector sequence set forth in SEQ ID NO: 17 and an effector complement sequence set forth in SEQ ID NO: 16 (shmiR3); a nucleic acid comprising or consisting of a DNA sequence encoding a shmiR comprising an effector sequence set forth in SEQ ID NO: 21 and an effector complement sequence set forth in SEQ ID NO: 20 (shmiR5); a nucleic acid comprising or consisting of a DNA sequence encoding a shmiR comprising an effector sequence set forth in SEQ ID NO: 27 and an effector complement sequence set forth in SEQ ID NO: 26 (shmiR9); a nucleic acid comprising or consisting of a DNA sequence encoding a shmiR comprising an effector sequence set forth in SEQ ID NO: 31 and an effector complement sequence set forth in SEQ ID NO: 30 (shmiR13); a nucleic acid comprising or consisting of a DNA sequence encoding a shmiR comprising an effector sequence set forth in SEQ ID NO: 33 and an effector complement sequence set forth in SEQ ID NO: 32 (shmiR14); and a nucleic acid comprising or consisting of a DNA sequence encoding a shmiR comprising an effector sequence set forth in SEQ ID NO: 39 and an effector complement sequence set forth in SEQ ID NO: 38 (shmiR17).

In one example, the at least two nucleic acids are selected from the group consisting of: a nucleic acid comprising or consisting of a DNA sequence set forth in SEQ ID NO: 56 (shmiR2); a nucleic acid comprising or consisting of a DNA sequence set forth in SEQ ID NO: 57 (shmiR3); a nucleic acid comprising or consisting of a DNA sequence set forth in SEQ ID NO: 59 (shmiR5); a nucleic acid comprising or consisting of a DNA sequence set forth in SEQ ID NO: 62 (shmiR9); a nucleic acid comprising or consisting of a DNA sequence set forth in SEQ ID NO: 64 (shmiR13); a nucleic acid comprising or consisting of a DNA sequence set forth in SEQ ID NO: 65 (shmiR14); and a nucleic acid comprising or consisting of a DNA sequence set forth in SEQ ID NO: 68 (shmiR17).

In one example, each of the at least two nucleic acids encode a shmiR comprising an effector sequence which is substantially complementary to a region of corresponding length in an RNA transcript set forth in one of SEQ ID NOs: 2, 9, 10 and 13. For example, the at least two nucleic acids may be selected from the group consisting of: a nucleic acid comprising or consisting of a DNA sequence encoding a shmiR comprising an effector sequence set forth in SEQ ID NO: 17 and an effector complement sequence set forth in SEQ ID NO: 16 (shmiR3); a nucleic acid comprising or consisting of a DNA sequence encoding a shmiR comprising an effector sequence set forth in SEQ ID NO: 31 and an effector complement sequence set forth in SEQ ID NO: 30 (shmiR13); a nucleic acid comprising or consisting of a DNA sequence encoding a shmiR comprising an effector sequence set forth in SEQ ID NO: 33 and an effector complement sequence set forth in SEQ ID NO: 32 (shmiR14); and a nucleic acid comprising or consisting of a DNA sequence encoding a shmiR comprising an effector sequence set forth in SEQ ID NO: 39 and an effector complement sequence set forth in SEQ ID NO: 38 (shmiR17).

In one example, the at least two nucleic acids are selected from the group consisting of: a nucleic acid comprising or consisting of a DNA sequence set forth in SEQ ID NO: 57 (shmiR3); a nucleic acid comprising or consisting of a DNA sequence set forth in SEQ ID NO: 64 (shmiR13); a nucleic acid comprising or consisting of a DNA sequence set forth in SEQ ID NO: 65 (shmiR14); and a nucleic acid comprising or consisting of a DNA sequence set forth in SEQ ID NO: 68 (shmiR17).

In one example, the at least two nucleic acids or ddRNAi construct comprising same comprises:

(a) a nucleic acid comprising or consisting of a DNA sequence encoding a shmiR comprising an effector sequence set forth in SEQ ID NO: 31 and an effector complement sequence set forth in SEQ ID NO: 30 (shmiR13); and

(b) a nucleic acid comprising or consisting of a DNA sequence encoding a shmiR comprising an effector sequence set forth in SEQ ID NO: 39 and an effector complement sequence set forth in SEQ ID NO: 38 (shmiR17).

In one example, the at least two nucleic acids or ddRNAi construct comprising same

(a) a nucleic acid comprising or consisting of the DNA sequence set forth in SEQ ID NO: 64 (shmiR13); and

(b) a nucleic acid comprising or consisting of the DNA sequence set forth in SEQ ID NO: 68 (shmiR17).

The composition described in any example hereof may further comprises one or more pharmaceutically acceptable carriers.

In some example, wherein the pharyngeal muscle comprises one or more of a inferior constricor muscle, a middle constrictor muscle, a superior constritor muscle, a palatopharyngeus muscle, a salpingopharyngeus muscle, a stylopharyngeus muscle, or any combination thereof.

According to one particular example, the present disclosure provides methods for administering to a pharyngeal muscle of a subject in need thereof a DNA construct comprising:

(a) a ddRNAi construct as described herein; and

(b) a PABPN1 construct comprising a DNA sequence encoding a functional PABPN1 protein having a mRNA transcript which is not targeted by the shmiR(s) encoded by the ddRNAi construct. Preferably, the DNA sequence encoding the functional PABPN1 protein is codon optimised such that its mRNA transcript is not targeted by the shmiRs of the ddRNAi construct. In one example, functional PABPN1 protein is a wild-type human PABPN1 protein e.g., having a sequence set forth in SEQ ID NO: 74. In one example a codon optimised DNA sequence encoding the functional PABPN1 protein is set forth in SEQ ID NO: 73. In some embodiments, the DNA construct can comprise one or more promoters. Exemplary promoters for use in the DNA constructs of the disclosure are muscle-specific promoter, such as for example, Spc512 and CK8. In some embodiments, the DNA construct comprises a promoter which is operably-linked to the PABPN1 construct and the ddRNAi construct, wherein the promoter is positioned upstream of the PABPN1 construct and the ddRNAi construct.

In some embodiments, the DNA construct comprises, in a 5′ to 3′ direction:

(a) a muscle-specific promoter e.g., Spc512;

(b) a PABPN1 construct as described herein comprising a DNA sequence encoding a functional PABPN1 protein having a mRNA transcript which is not targeted by the shmiRs encoded by the ddRNAi construct; and

(c) a ddRNAi construct of the disclosure comprising a nucleic acid comprising a DNA sequence encoding shmiR13 as described herein and a nucleic acid comprising a DNA sequence encoding shmiR17 as described herein.

In some embodiments, the pharyngeal muscle comprises one or more of a inferior constricor muscle, a middle constrictor muscle, a superior constritor muscle, a palatopharyngeus muscle, a salpingopharyngeus muscle, a stylopharyngeus muscle, or any combination thereof.

### BRIEF DESCRIPTION OF DRAWINGS

FIG. 1A is a schematic illustrating a construct for simultaneous gene silencing of endogenous PABPN1 and replacement with codon optimised PABPN1 generated by subcloning two shmiRs targeting wtPABPN1 into the 3′ untranslated region of the codon optimized PABPN1 transcript in between two pAAV2 ITRs.

FIG. 1B is a schematic illustrating the ‘silence and replace’ construct (SR-construct) designed for simultaneous gene silencing of endogenous PABPN1 and replacement with codon optimised PABPN1 generated by subcloning two shmiRs targeting wtPABPN1 (shmiR17 and shmiR13) into the 3′ untranslated region of the codon optimized PABPN1 transcript in the pAAV2 vector backbone.

FIG. 1C illustrates the predicted secondary structure of a representative shmiR construct comprising a 5′ flanking region, a siRNA sense strand; a stem/loop junction sequence, an siRNA anti-sense strand, and a 3′ flanking region.

FIG. 2 is a schematic illustrating a SR-construct. In the SR-construct, the ‘replace’ and ‘silence’ cassettes are all inserted in a single vector with the Spc512 muscle specific promoter. Two shmiR sequences are inserted in the 3′UTR of the codon-optimised PABPN1 cassette.

FIG. 3A shows expression of shRNA in (Tibialis anterior) TA muscles of A17 mice injected with the SR-construct. RNA was extracted from TA samples 14 weeks post SR-construct dosing.

FIG. 3B shows silencing of PABPN1 expression (including expPABPN1) in TA muscles of A17 mice treated with the SR-construct. RNA was extracted from TA samples 14 weeks post SR-construct dosing.

FIG. 3C illustrates restoration of normal PABPN1 levels in the A17 mouse model upon treatment with the SR-construct. RNA was extracted from TA muscle samples 14 weeks post SR-construct dosing.

FIG. 4A shows significantly reduced formation of insoluble aggregates (intranuclear inclusions (INIs)) containing PABPN1 with a SR-construct dose effect. The SR-construct was injected in TA muscles of A17 mice. Muscles were collected and mounted for histological studies 14 weeks post SR-construct dosing. Immunofluorescence for PABPN1 is shown in green and immunofluorescence for Laminin is shown in red.

FIG. 4B shows quantification of percentage of nuclei containing INIs in muscle sections indicating that treatment with the SR-construct significantly reduces the amount of INIs compared to untreated A17 TA muscles (one-way Anova test with Bonferroni post-doc test, ***p<0.001, ns: not significant).

FIG. 5A shows a significant increase in the maximal force generated by TA muscles of A17 mice in an SR-construct dose-dependent manner. Maximal force was measured by in situ muscle physiology.

FIG. 5B shows muscle weight normalized to body weight (BW) of SR-construct-treated TA muscles of A17 mice. Normalized muscle weight was comparable to that of control FvB mice at doses above 1e10 vg per TA injected (mean±SEM n=10, One-way Anova test with Bonferroni post-doc test, *p<0.05, ***p<0.001, **p<0.01, ns: not significant).

FIG. 6A shows maximal force generated by TA muscles of A17 mice 14 weeks post SR-construct dosing. Maximal force was measured by in situ muscle physiology.

FIG. 6B shows maximal force generated by TA muscles of A17 mice 20 weeks post SR-construct dosing. Maximal force was measured by in situ muscle physiology.

FIG. 7A shows direct injection of the SR-construct into pharyngeal muscles of sheep.

FIG. 7B shows radio images using a radiolableld cream illustrating severe dysphagia in human OPMD patients with risk of “fausse route.”

### KEY TO THE SEQUENCE LISTING

SEQ ID NO: 1: RNA sequence for region within mRNA transcript corresponding to PABPN1 protein designated PABPN1 mRNA Region 2.
SEQ ID NO: 2: RNA sequence for region within mRNA transcript corresponding to PABPN1 protein designated PABPN1 mRNA Region 3.
SEQ ID NO: 3: RNA sequence for region within mRNA transcript corresponding to PABPN1 protein designated PABPN1 mRNA Region 4.
SEQ ID NO: 4: RNA sequence for region within mRNA transcript corresponding to PABPN1 protein designated PABPN1 mRNA Region 5.
SEQ ID NO: 5: RNA sequence for region within mRNA transcript corresponding to PABPN1 protein designated PABPN1 mRNA Region 6.
SEQ ID NO: 6: RNA sequence for region within mRNA transcript corresponding to PABPN1 protein designated PABPN1 mRNA Region 7.
SEQ ID NO: 7: RNA sequence for region within mRNA transcript corresponding to PABPN1 protein designated PABPN1 mRNA Region 9.
SEQ ID NO: 8: RNA sequence for region within mRNA transcript corresponding to PABPN1 protein designated PABPN1 mRNA Region 11.
SEQ ID NO: 9: RNA sequence for region within mRNA transcript corresponding to PABPN1 protein designated PABPN1 mRNA Region 13.
SEQ ID NO: 10: RNA sequence for region within mRNA transcript corresponding to PABPN1 protein designated PABPN1 mRNA Region 14.
SEQ ID NO: 11: RNA sequence for region within mRNA transcript corresponding to PABPN1 protein designated PABPN1 mRNA Region 15.
SEQ ID NO: 12: RNA sequence for region within mRNA transcript corresponding to PABPN1 protein designated PABPN1 mRNA Region 16.
SEQ ID NO: 13: RNA sequence for region within mRNA transcript corresponding to PABPN1 protein designated PABPN1 mRNA Region 17.
SEQ ID NO: 14: RNA effector complement sequence for shmiR designated shmiR2.
SEQ ID NO: 15: RNA effector sequence for shmiR designated shmiR2.
SEQ ID NO: 16: RNA effector complement sequence for shmiR designated shmiR3.
SEQ ID NO: 17: RNA effector sequence for shmiR designated shmiR3.
SEQ ID NO: 18: RNA effector complement sequence for shmiR designated shmiR4.
SEQ ID NO: 19: RNA effector sequence for shmiR designated shmiR4.
SEQ ID NO: 20: RNA effector complement sequence for shmiR designated shmiR5.
SEQ ID NO: 21: RNA effector sequence for shmiR designated shmiR5.
SEQ ID NO: 22: RNA effector complement sequence for shmiR designated shmiR6.
SEQ ID NO: 23: RNA effector sequence for shmiR designated shmiR6.
SEQ ID NO: 24: RNA effector complement sequence for shmiR designated shmiR7.
SEQ ID NO: 25: RNA effector sequence for shmiR designated shmiR7.
SEQ ID NO: 26: RNA effector complement sequence for shmiR designated shmiR9.
SEQ ID NO: 27: RNA effector sequence for shmiR designated shmiR9.
SEQ ID NO: 28: RNA effector complement sequence for shmiR designated shmiR11.
SEQ ID NO: 29: RNA effector sequence for shmiR designated shmiR11.
SEQ ID NO: 30: RNA effector complement sequence for shmiR designated shmiR13.
SEQ ID NO: 31: RNA effector sequence for shmiR designated shmiR13.
SEQ ID NO: 32: RNA effector complement sequence for shmiR designated shmiR14.
SEQ ID NO: 33: RNA effector sequence for shmiR designated shmiR14.
SEQ ID NO: 34: RNA effector complement sequence for shmiR designated shmiR15.
SEQ ID NO: 35: RNA effector sequence for shmiR designated shmiR15.
SEQ ID NO: 36: RNA effector complement sequence for shmiR designated shmiR16.
SEQ ID NO: 37: RNA effector sequence for shmiR designated shmiR16.
SEQ ID NO: 38: RNA effector complement sequence for shmiR designated shmiR17.
SEQ ID NO: 39: RNA effector sequence for shmiR designated shmiR17.
SEQ ID NO: 40: RNA stem loop sequence for shmiRs
SEQ ID NO: 41: 5′ flanking sequence of the pri-miRNA backbone.
SEQ ID NO: 42: 3′ flanking sequence of the pri-miRNA backbone
SEQ ID NO: 43: RNA sequence for shmiR designated shmiR2.
SEQ ID NO: 44: RNA sequence for shmiR designated shmiR3.
SEQ ID NO: 45: RNA sequence for shmiR designated shmiR4.
SEQ ID NO: 46: RNA sequence for shmiR designated shmiR5.
SEQ ID NO: 47: RNA sequence for shmiR designated shmiR6.
SEQ ID NO: 48: RNA sequence for shmiR designated shmiR7.
SEQ ID NO: 49: RNA sequence for shmiR designated shmiR9.
SEQ ID NO: 50: RNA sequence for shmiR designated shmiR11.
SEQ ID NO: 51: RNA sequence for shmiR designated shmiR13.
SEQ ID NO: 52: RNA sequence for shmiR designated shmiR14.
SEQ ID NO: 53: RNA sequence for shmiR designated shmiR15.
SEQ ID NO: 54: RNA sequence for shmiR designated shmiR16.
SEQ ID NO: 55: RNA sequence for shmiR designated shmiR17.
SEQ ID NO: 56: DNA sequence coding for shmiR designated shmiR2.
SEQ ID NO: 57: DNA sequence coding for shmiR designated shmiR3.
SEQ ID NO: 58: DNA sequence coding for shmiR designated shmiR4.
SEQ ID NO: 59: DNA sequence coding for shmiR designated shmiR5.
SEQ ID NO: 60: DNA sequence coding for shmiR designated shmiR6.
SEQ ID NO: 61: DNA sequence coding for shmiR designated shmiR7.
SEQ ID NO: 62: DNA sequence coding for shmiR designated shmiR9.
SEQ ID NO: 63: DNA sequence coding for shmiR designated shmiR11.
SEQ ID NO: 64: DNA sequence coding for shmiR designated shmiR13.
SEQ ID NO: 65: DNA sequence coding for shmiR designated shmiR14.
SEQ ID NO: 66: DNA sequence coding for shmiR designated shmiR15.
SEQ ID NO: 67: DNA sequence coding for shmiR designated shmiR16.
SEQ ID NO: 68: DNA sequence coding for shmiR designated shmiR17.
SEQ ID NO: 69: DNA sequence for double construct version 1 coding for shmiR3 and shmiR14 under control of the muscle specific CK8 promoter and codon optimized PABPN1 under control of Spc512
SEQ ID NO: 70: DNA sequence for double construct version 1 coding for shmiR17 and shmiR13 under control of the muscle specific CK8 promoter and codon optimized PABPN1 under control of Spc512
SEQ ID NO: 71: DNA sequence for double construct version 2 coding for coPABPN1 and shmiRs designated shmiR3 and shmiR14, under control of Spc512.
SEQ ID NO: 72: DNA sequence for double construct version 2 coding for coPABPN1 and shmiRs designated shmiR17 and shmiR13 under control of Spc512.
SEQ ID NO: 73 DNA sequence for Human codon-optimized PABPN1 cDNA sequence.
SEQ ID NO: 74 Amino acid sequence for codon-optimised human PABPN1 protein.
SEQ ID NO: 75 Amino acid sequence for wildtype human PABPN1 protein with FLAG-tag.
SEQ ID NO: 76 Amino acid sequence for codon-optimised human PABPN1 protein with FLAG-tag.
SEQ ID NO: 77 DNA sequence for primer designated wtPABPN1-Fwd.
SEQ ID NO: 78 DNA sequence for primer designated wtPABPN1-Rev
SEQ ID NO: 79 DNA sequence for probe designated wtPABPN1-Probe
SEQ ID NO: 80 DNA sequence for primer designated optPABPN1-Fwd
SEQ ID NO: 81 DNA sequence for primer designated optPABPN1-Rev
SEQ ID NO: 82 DNA sequence for probe designated optPABPN1-Probe
SEQ ID NO: 83 DNA sequence for primer designated shmiR3-FWD
SEQ ID NO: 84 DNA sequence for primer designated shmiR13-FWD
SEQ ID NO: 85 DNA sequence for primer designated shmiR14-FWD
SEQ ID NO: 86 DNA sequence for primer designated shmiR17-FWD
SEQ ID NO: 87 RNA sequence encoding wildtype human PABPN1 protein
SEQ ID NO: 88 Consensus sequence for modified phospholipase A2 (PLA2) domain of AAV VP1
SEQ ID NO: 89 Modified PLA2 domain for AAV8
SEQ ID NO: 90 Modified PLA2 domain for AAV9

### DETAILED DESCRIPTION

### General

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

### Selected Definitions

By “RNA” is meant a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA.

The term “RNA interference” or “RNAi” refers generally to RNA-dependent silencing of gene expression initiated by double stranded RNA (dsRNA) molecules in a cell's cytoplasm. The dsRNA molecule reduces or inhibits transcription products of a target nucleic acid sequence, thereby silencing the gene or reducing expression of that gene.

As used herein, the term “double stranded RNA” or “dsRNA” refers to a RNA molecule having a duplex structure and comprising an effector sequence and an effector complement sequence which are of similar length to one another. The effector sequence and the effector complement sequence can be in a single RNA strand or in separate RNA strands. The “effector sequence” (often referred to as a “guide strand”) is substantially complementary to a target sequence, which in the present case, is a region of a PABPN1 mRNA transcript. The “effector sequence” can also be referred to as the “antisense sequence”. The “effector complement sequence” will be of sufficient complementary to the effector sequence such that it can anneal to the effector sequence to form a duplex. In this regard, the effector complement sequence will be substantially homologous to a region of target sequence. As will be apparent to the skilled person, the term “effector complement sequence” can also be referred to as the “complement of the effector sequence” or the sense sequence.

As used herein, the term “duplex” refers to regions in two complementary or substantially complementary nucleic acids (e.g., RNAs), or in two complementary or substantially complementary regions of a single-stranded nucleic acid (e.g., RNA), that form base pairs with one another, either by Watson-Crick base pairing or any other manner that allows for a stabilized duplex between the nucleotide sequences that are complementary or substantially complementary. It will be understood by the skilled person that within a duplex region, 100% complementarity is not required; substantial complementarity is allowable. Substantial complementarity includes may include 79% or greater complementarity. For example, a single mismatch in a duplex region consisting of 19 base pairs (i.e., 18 base pairs and one mismatch) results in 94.7% complementarity, rendering the duplex region substantially complementary. In another example, two mismatches in a duplex region consisting of 19 base pairs (i.e., 17 base pairs and two mismatches) results in 89.5% complementarity, rendering the duplex region substantially complementary. In yet another example, three mismatches in a duplex region consisting of 19 base pairs (i.e., 16 base pairs and three mismatches) results in 84.2% complementarity, rendering the duplex region substantially complementary, and so on. The dsRNA may be provided as a hairpin or stem loop structure, with a duplex region comprised of an effector sequence and effector complement sequence linked by at least 2 nucleotide sequence which is termed a stem loop. When a dsRNA is provided as a hairpin or stem loop structure it can be referred to as a “hairpin RNA” or “short hairpin RNAi agent” or “shRNA”. Other dsRNA molecules provided in, or which give rise to, a hairpin or stem loop structure include primary miRNA transcipts (pri-miRNA) and precursor microRNA (pre-miRNA). Pre-miRNA shRNAs can be naturally produced from pri-miRNA by the action of the enzymes Drosha and Pasha which recognize and release regions of the primary miRNA transcript which form a stem-loop structure. Alternatively, the pri-miRNA transcript can be engineered to replace the natural stem-loop structure with an artificial/recombinant stem-loop structure. That is, an artificial/recombinant stem-loop structure may be inserted or cloned into a pri-miRNA backbone sequence which lacks its natural stem-loop structure. In the case of stemloop sequences engineered to be expressed as part of a pri-miRNA molecule, Drosha and Pasha recognize and release the artificial shRNA. dsRNA molecules produced using this approach are known as “shmiRNAs”, “shmiRs” or “microRNA framework shRNAs”.

As used herein, the term “complementary” with regard to a sequence refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence. A person of skill in the art will also recognise that a G present in RNA may pair with C or U in RNA.

As used herein, the term “substantially complementary” is used to indicate a sufficient degree of complementarity or precise pairing such that stable and specific binding occurs between nucleic acid sequences e.g., between the effector sequence and the effector complement sequence or between the effector sequence and the target sequence. It is understood that the sequence of a nucleic acid need not be 100% complementary to that of its target or complement. The term encompasses a sequence complementary to another sequence with the exception of an overhang. In some cases, the sequence is complementary to the other sequence with the exception of 1-2 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4 mismatches.

The term “encoded”, as used in the context of a shRNA or shmiR of the disclosure, shall be understood to mean a shRNA or shmiR which is capable of being transcribed from a DNA template. Accordingly, a nucleic acid that encodes, or codes for, a shRNA or shmiR of the disclosure will comprise a DNA sequence which serves as a template for transcription of the respective shRNA or shmiR.

The term “DNA-directed RNAi construct” or “ddRNAi construct” refers to a nucleic acid comprising DNA sequence which, when transcribed produces a shRNA or shmiR molecule (preferably a shmiR) which elicits RNAi. The ddRNAi construct may comprise a nucleic acid which is transcribed as a single RNA that is capable of self-annealing into a hairpin structure with a duplex region linked by a stem loop of at least 2 nucleotides i.e., shRNA or shmiR, or as a single RNA with multiple shRNAs or shmiRs, or as multiple RNA transcripts each capable of folding as a single shRNA or shmiR respectively. The ddRNAi construct may be provided within a larger “DNA construct” comprising one or more additional DNA sequences. For example, the ddRNAi construct may be provided in a DNA construct comprising a further DNA sequence coding for functional PABPN1 protein which has been codon optimised such that its mRNA transcript is not targeted by shmiRs of the ddRNAi construct. The ddRNAi construct and/or the DNA construct comprising same may be within an expression vector e.g., operably linked to a promoter.

As used herein, the term “operably-linked” or “operable linkage” (or similar) means that a coding nucleic acid sequence is linked to, or in association with, a regulatory sequence, e.g., a promoter, in a manner which facilitates expression of the coding sequence. Regulatory sequences include promoters, enhancers, and other expression control elements that are art-recognized and are selected to direct expression of the coding sequence.

A “vector” will be understood to mean a vehicle for introducing a nucleic acid into a cell. Vectors include, but are not limited to, plasmids, phagemids, viruses, bacteria, and vehicles derived from viral or bacterial sources, A “plasmid” is a circular, double-stranded DNA molecule. A useful type of vector for use in accordance with the present disclosure is a viral vector, wherein heterologous DNA sequences are inserted into a viral genome that can be modified to delete one or more viral genes or parts thereof. Certain vectors are capable of autonomous replication in a host cell (e.g., vectors having an origin of replication that functions in the host cell). Other vectors can be stably integrated into the genome of a host cell, and are thereby replicated along with the host genome. As used herein, the term “expression vector” will be understood to mean a vector capable of expressing a RNA molecule of the disclosure.

A “functional PABPN1 protein” shall be understood to mean a PABPN1 protein having the functional properties of a wild-type PABPN1 protein e.g., an ability to control site of mRNA polyadenylation and/or intron splicing in a mammalian cell. Accordingly, a “functional PABPN1 protein” will be understood to be a PABPN1 protein which is not causative of OPMD when expressed or present in a subject. In one example, a reference herein to “functional PABPN1 protein” is a reference to human wild-type PABPN1 protein. The sequence of human wild-type PABPN1 protein is set forth in NCBI RefSeq NP_004634. Accordingly, a functional human PABPN1 protein may have the functional properties in vivo of the human PABPN1 protein set forth in NCBI RefSeq NP_004634.

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, ameliorating or palliating the disease state, and remission or improved prognosis. It follows that treatment of OPMD includes reducing or inhibiting expression of a PABPN1 protein which is causative of OPMD in the subject and/or expressing in the subject a PABPN1 protein having the normal length of polyalanine residues. Preferably, treatment of OPMD includes reducing or inhibiting expression of the PABPN1 protein which is causative of OPMD in the subject and expressing in the subject a PABPN1 protein having the normal length of polyalanine residues. An individual is successfully “treated”, for example, if one or more of the above treatment outcomes is achieved.

A “therapeutically effective amount” is at least the minimum concentration or amount required to effect a measurable improvement in the OPMD condition, such as a measurable improvement in in one or more symptoms of OPMD e.g., including but not limited to ptosis, dysphagia and muscle weakness in the subject. A therapeutically effective amount herein may vary according to factors such as the disease state, age, sex, and weight of the patient, and the ability of the shmiR, nucleic acid encoding same, ddRNAi construct, DNA construct, expression vector, or composition comprising same, to elicit a desired response in the individual and/or the ability of the expression vector to express functional PABPN1 protein in the subject. A therapeutically effective amount is also one in which any toxic or detrimental effects of the shmiR, nucleic acid encoding same, ddRNAi construct, DNA construct, expression vector, or composition comprising same, are outweighed by the therapeutically beneficial effects of the shmiR, nucleic acid encoding same, ddRNAi construct, DNA construct, expression vector, or composition comprising same, to inhibit, supress or reduce expression of PABPN1 protein causative of OPMD considered alone or in combination with the therapeutically beneficial effects of the expression of functional PABPN1 protein in the subject.

As used herein, the “subject” or “patient” can be a human or non-human animal suffering from or genetically predisposed to OPMD i.e., possess a PABPN1 gene variant which is causative of OPMD. The “non-human animal” may be a primate, livestock (e.g. sheep, horses, cattle, pigs, donkeys), companion animal (e.g. pets such as dogs and cats), laboratory test animal (e.g. mice, rabbits, rats, guinea pigs, drosophila, C. elegans, zebrafish), performance animal (e.g. racehorses, camels, greyhounds) or captive wild animal. In one example, the subject or patient is a mammal. In one example, the subject or patient is a human.

The terms “reduced expression”, “reduction in expression” or similar, refer to the absence or an observable decrease in the level of protein and/or mRNA product from the target gene e.g., the PABPN1 gene. The decrease does not have to be absolute, but may be a partial decrease sufficient for there to a detectable or observable change as a result of the RNAi effected by the shmiR, nucleic acid encoding same, ddRNAi construct, DNA construct, expression vector, or composition comprising same of the disclosure. The decrease can be measured by determining a decrease in the level of mRNA and/or protein product from a target nucleic acid relative to a cell lacking the shmiR, nucleic acid encoding same, ddRNAi construct, DNA construct, expression vector, or composition comprising same, and may be as little as 1%, 5% or 10%, or may be absolute i.e., 100% inhibition. The effects of the decrease may be determined by examination of the outward properties i.e., quantitative and/or qualitative phenotype of the cell or organism, and may also include detection of the presence or a change in the amount of nuclear aggregates of expPABPN1 in the cell or organism following administration of a shmiR, nucleic acid encoding same, ddRNAi construct, DNA construct, expression vector, or composition comprising same, of the disclosure.

A “delivery system” as used herein refers to a vector for packaging foreign genetic material, such as DNA or RNA, and which can be introduced into a cell. Delivery systems can include viral vectors, e.g., an adeno-associated viral (AAV) vector, a retroviral vector, an adenoviral vector (AdV) and a lentiviral (LV) vector. As described herein, viral vectors can be used to deliver and express foreign genetic material in cell. Accordingly, a viral expression vector as described herein may be used as a delivery system.

A “pharyngeal muscle” as used herein refers to one or more of the group of muscles that form the pharynx. The pharyngeal muscle can include one or more of the inferior constricor muscle, middle constrictor muscle, superior constritor muscle, palatopharyngeus muscle, the salpingopharyngeus muscle, and/or the stylopharyngeus muscle.

### Methods of Treatment

Certain aspects of the disclosure are directed to administering to a human subject in need thereof one or more nucleic acid(s), ddRNAi construct(s), DNA constructs, expression vector(s), delivery system(s), or composition(s) comprising same as described herein be used for treating the subject and/or inhibiting expression of endogenous PABPN1 protein, including a PABPN1 protein which is causative of OPMD, in the subject, wherein the composition is administered by direct injection to a pharyngeal muscle of the subject.

In some embodiments, one or more nucleic acid(s), ddRNAi construct(s), DNA construct(s), expression vector(s), delivery system(s), or composition(s) comprising same as described herein may be used to treat OPMD in a subject suffering therefrom. Similarly, one or more nucleic acid(s), ddRNAi construct(s), DNA construct(s), expression vector(s), delivery system(s), or composition(s) comprising same as described herein may be used to prevent the development or progression of one or more symptoms of OPMD in a subject suffering therefrom or predisposed thereto.

In some embodiments, the subject has improved swallowing following administering one or more nucleic acid(s), ddRNAi construct(s), DNA construct(s), expression vector(s), delivery system(s), or composition(s) comprising same as described herein by direct injection to a pharyngeal muscle of the subject.

In certain embodiments, the expression vector and/or composition of the disclosure may comprise both a ddRNAi construct of the disclosure and a codon-optimised nucleic acid encoding functional PABPN1 protein of the disclosure. Accordingly, administration of the expression vector or composition may be effective to (i) inhibit, reduce or knockdown expression of endogenous PABPN1, including the PABPN1 protein comprising an expanded polyalanine tract which is causative of OPMD, and (ii) provide for expression of a functional PABPN1 protein which is not targeted by shmiRs or shRNAs which inhibit, reduce or knockdown expression of endogenous PABPN1. A composition of the disclosure may thus restore PABPN1 protein function, e.g., post-transcriptional processing of RNA, in a cell or animal to which it is administered.

In certain embodiments, treatment of OPMD may comprise administering by direct injection to a pharyngeal muscle of a subject separately to the subject (i) one or more agents for inhibiting expression of a PABPN1 protein which is causative of OPMD, and (ii) an expression vector comprising a codon-optimised nucleic acid encoding functional PABPN1 protein of the disclosure or composition comprising same. As described herein, the one or more agents for inhibiting expression of a PABPN1 protein which is causative of OPMD may be a nucleic acid, a ddRNAi construct, an expression vector or composition comprising same as described herein or a plurality of any one or more thereof. The subject may be administered components (i) and (ii) together, simultaneously or consecutively.

In some embodiments, treatment of OPMD may comprise administering by direct injection to a pharyngeal muscle of the subject a codon-optimised nucleic acid encoding a functional PABPN1 protein of the disclosure, wherein the subject has previously been administered one or more agents for inhibiting expression of a PABPN1 protein which is causative of OPMD but which does not inhibit expression of the codon-optimised nucleic acid. For example, the subject may have been previously administered a nucleic acid, a ddRNAi construct, an expression vector or composition comprising same as described herein or a plurality of any one or more thereof.

In some embodiments, the route of administration is IM (e.g., direct injection to a pharyngeal muscle of the subject) and achieves effective delivery to muscle tissue and transfection of a ddRNAi constructs and/or codon-optimised nucleic acids encoding PABPN1 of the disclosure, and expression of shmiRs or shRNA and/or the codon-optimised nucleic acid therein.

The therapeutically effective dose level for any particular patient will depend upon a variety of factors including: the composition employed; the age, body weight, general health, sex and diet of the patient; the time of administration; the route of administration; the rate of sequestration of the nucleic acid, a ddRNAi construct, a DNA construct, an expression vector or composition comprising same as described herein, or a plurality of any one or more thereof, the duration of the treatment, together with other related factors.

Efficacy of a nucleic acid, a ddRNAi construct, a DNA construct, an expression vector, delivery system, or composition comprising same of the disclosure to reduce or inhibit expression of the PABPN1 protein causative of OPMD and to express functional PABPN1 protein which is not causative of OPMD in an amount sufficient to restore PABPN1 function, may be determined by evaluating muscle contractile properties and/or swallowing difficulties in the subject treated. Methods for testing swallowing ability and muscle contractile properties are known in the art. For example, swallowing difficulties may be evaluated using videofluoroscopy, UGI endoscopy or oesophageal manometry and impedance testing. Other methods for assessing clinical features of OPMD are described in Rüegg et al., (2005) Swiss Medical Weekly, 135:574-586.

### Agents for RNAi

As described herein, a nucleic acid useful in a method of the disclosure comprises a DNA sequence which encodes a short hairpin micro-RNA (shmiR) which targets a region of the messenger RNA transcript of human PABPN1, wherein the shmiR comprises:

an effector sequence of at least 17 nucleotides in length;

an effector complement sequence;

a stemloop sequence; and

primary micro RNA (pri-miRNA) backbone;

wherein the effector sequence is substantially complementary to a region of corresponding length within the RNA transcript of human PABPN1. For example, the effector sequence may be substantially complementary to a region of corresponding length within the sequence set forth in SEQ ID NO: 87. In some examples, the present disclosure provides a nucleic acid comprising a DNA sequence which encodes a shmiR, said shmiR comprising:

an effector sequence of at least 17 nucleotides in length;

an effector complement sequence;

a stemloop sequence; and

pri-miRNA backbone;

wherein the effector sequence is substantially complementary to a region of corresponding length in an RNA transcript set forth in any one of SEQ ID NOs: 1-13. Preferably, the effector sequence will be less than 30 nucleotides in length. For example, a suitable effector sequence may be in the range of 17-29 nucleotides in length. In a particularly preferred example, the effector sequence will be 21 nucleotides in length. More preferably, the effector sequence will be 21 nucleotides in length and the effector complement sequence will be 20 nucleotides in length.

In certain embodiments, the shmiR comprises an effector sequence which is substantially complementary to a region of corresponding length in an RNA transcript comprising or consisting of the sequence set forth in any one of SEQ ID NOs: 1-13 (i.e., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13). For example, the effector sequence may be substantially complementary to a region of corresponding length in an RNA transcript comprising or consisting of the sequence set forth in any one of SEQ ID NOs: 1-13 and contain 4 mismatch bases relative thereto. For example, the effector sequence may be substantially complementary to a region of corresponding length in an RNA transcript comprising or consisting of the sequence set forth in any one of SEQ ID NOs: 1-13 and contain 3 mismatch bases relative thereto. For example, the effector sequence may be substantially complementary to a region of corresponding length in an RNA transcript comprising or consisting of the sequence set forth in any one of SEQ ID NOs: 1-13 and contain 2 mismatch bases relative thereto. For example, the effector sequence may be substantially complementary to a region of corresponding length in an RNA transcript comprising or consisting of the sequence set forth in any one of SEQ ID NOs: 1-13 and contain 1 mismatch base relative thereto. For example, the effector sequence may be 100% complementary to a region of corresponding length in an RNA transcript comprising or consisting of the sequence set forth in any one of SEQ ID NOs: 1-13.

In one example, the shmiR comprises an effector sequence which is substantially complementary to a region of corresponding length in an RNA transcript comprising or consisting of the sequence set forth in SEQ ID NO: 9. For example, the effector sequence may be substantially complementary to a region of corresponding length in an RNA transcript comprising or consisting of the sequence set forth in SEQ ID NO: 9 and contain 4 mismatch bases relative thereto. For example, the effector sequence may be substantially complementary to a region of corresponding length in an RNA transcript comprising or consisting of the sequence set forth in SEQ ID NO: 9 and contain 3 mismatch bases relative thereto. For example, the effector sequence may be substantially complementary to a region of corresponding length in an RNA transcript comprising or consisting of the sequence set forth in SEQ ID NO: 9 and contain 2 mismatch bases relative thereto. For example, the effector sequence may be substantially complementary to a region of corresponding length in an RNA transcript comprising or consisting of the sequence set forth in SEQ ID NO: 9 and contain 1 mismatch base relative thereto. For example, the effector sequence may be 100% complementary to a region of corresponding length in an RNA transcript comprising or consisting of the sequence set forth in SEQ ID NO: 9.

In one example, the shmiR comprises an effector sequence which is substantially complementary to a region of corresponding length in an RNA transcript comprising or consisting of the sequence set forth in SEQ ID NO: 13. For example, the effector sequence may be substantially complementary to a region of corresponding length in an RNA transcript comprising or consisting of the sequence set forth in SEQ ID NO: 13 and contain 4 mismatch bases relative thereto. For example, the effector sequence may be substantially complementary to a region of corresponding length in an RNA transcript comprising or consisting of the sequence set forth in SEQ ID NO: 13 and contain 3 mismatch bases relative thereto. For example, the effector sequence may be substantially complementary to a region of corresponding length in an RNA transcript comprising or consisting of the sequence set forth in SEQ ID NO: 13 and contain 2 mismatch bases relative thereto. For example, the effector sequence may be substantially complementary to a region of corresponding length in an RNA transcript comprising or consisting of the sequence set forth in SEQ ID NO: 13 and contain 1 mismatch base relative thereto. For example, the effector sequence may be 100% complementary to a region of corresponding length in an RNA transcript comprising or consisting of the sequence set forth in SEQ ID NO: 13.

In accordance with an example in which the effector sequence of a shmiR of the disclosure is substantially complementary to a region of corresponding length in a PABPN1 miRNA transcript described herein and contains 1, 2, 3 or 4 mismatch base(s) relative thereto, it is preferred that the mismatch(es) are not located within the region corresponding to the seed region of the shmiR i.e., nucleotides 2-8 of the effector sequence.

In some embodiments, the nucleic acid described herein may comprise a DNA sequence encoding a shmiR comprising: (i) an effector sequence which is substantially complementary to the sequence set forth in SEQ ID NO:14 with the exception of 1, 2, 3 or 4 base mismatches, provided that the effector sequence is capable of forming a duplex with a sequence set forth in SEQ ID NO:14; and (ii) an effector complement sequence comprising a sequence which is substantially complementary to the effector sequence. For example, the shmiR encoded by the nucleic acid may comprise an effector sequence set forth in SEQ ID NO:15 and an effector complement sequence which is substantially complementary to the sequence set forth in SEQ ID NO:15 and capable of forming a duplex therewith. The effector complement sequence which is substantially complementary to the sequence set forth in SEQ ID NO:15 may be the sequence set forth in SEQ ID NO:14. A shmiR in accordance with this example is hereinafter designated “shmiR2”.

In one example, the nucleic acid described herein may comprise a DNA sequence encoding a shmiR comprising: (i) an effector sequence which is substantially complementary to the sequence set forth in SEQ ID NO:16 with the exception of 1, 2, 3 or 4 base mismatches, provided that the effector sequence is capable of forming a duplex with a sequence set forth in SEQ ID NO:16; and (ii) an effector complement sequence comprising a sequence which is substantially complementary to the effector sequence. For example, the shmiR encoded by the nucleic acid may comprise an effector sequence set forth in SEQ ID NO:17 and an effector complement sequence which is substantially complementary to the sequence set forth in SEQ ID NO:17 and capable of forming a duplex therewith. The effector complement sequence which is substantially complementary to the sequence set forth in SEQ ID NO:17 may be the sequence set forth in SEQ ID NO:16. A shmiR in accordance with this example is hereinafter designated “shmiR3”.

In one example, the nucleic acid described herein may comprise a DNA sequence encoding a shmiR comprising: (i) an effector sequence which is substantially complementary to the sequence set forth in SEQ ID NO:18 with the exception of 1, 2, 3 or 4 base mismatches, provided that the effector sequence is capable of forming a duplex with a sequence set forth in SEQ ID NO:18; and (ii) an effector complement sequence comprising a sequence which is substantially complementary to the effector sequence. For example, the shmiR encoded by the nucleic acid may comprise an effector sequence set forth in SEQ ID NO:19 and an effector complement sequence which is substantially complementary to the sequence set forth in SEQ ID NO:19 and capable of forming a duplex therewith. The effector complement sequence which is substantially complementary to the sequence set forth in SEQ ID NO:19 may be the sequence set forth in SEQ ID NO:18. A shmiR in accordance with this example is hereinafter designated “shmiR4”.

In one example, the nucleic acid described herein may comprise a DNA sequence encoding a shmiR comprising: (i) an effector sequence which is substantially complementary to the sequence set forth in SEQ ID NO:20 with the exception of 1, 2, 3 or 4 base mismatches, provided that the effector sequence is capable of forming a duplex with a sequence set forth in SEQ ID NO:20; and (ii) an effector complement sequence comprising a sequence which is substantially complementary to the effector sequence. For example, the shmiR encoded by the nucleic acid may comprise an effector sequence set forth in SEQ ID NO:21 and an effector complement sequence which is substantially complementary to the sequence set forth in SEQ ID NO:21 and capable of forming a duplex therewith. The effector complement sequence which is substantially complementary to the sequence set forth in SEQ ID NO:21 may be the sequence set forth in SEQ ID NO:20. A shmiR in accordance with this example is hereinafter designated “shmiR5”.

In one example, the nucleic acid described herein may comprise a DNA sequence encoding a shmiR comprising: (i) an effector sequence which is substantially complementary to the sequence set forth in SEQ ID NO:22 with the exception of 1, 2, 3 or 4 base mismatches, provided that the effector sequence is capable of forming a duplex with a sequence set forth in SEQ ID NO:22; and (ii) an effector complement sequence comprising a sequence which is substantially complementary to the effector sequence. For example, the shmiR encoded by the nucleic acid may comprise an effector sequence set forth in SEQ ID NO:23 and an effector complement sequence which is substantially complementary to the sequence set forth in SEQ ID NO:23 and capable of forming a duplex therewith. The effector complement sequence which is substantially complementary to the sequence set forth in SEQ ID NO:23 may be the sequence set forth in SEQ ID NO:22. A shmiR in accordance with this example is hereinafter designated “shmiR6”.

In one example, the nucleic acid described herein may comprise a DNA sequence encoding a shmiR comprising: (i) an effector sequence which is substantially complementary to the sequence set forth in SEQ ID NO:24 with the exception of 1, 2, 3 or 4 base mismatches, provided that the effector sequence is capable of forming a duplex with a sequence set forth in SEQ ID NO:24; and (ii) an effector complement sequence comprising a sequence which is substantially complementary to the effector sequence. For example, the shmiR encoded by the nucleic acid may comprise an effector sequence set forth in SEQ ID NO:25 and an effector complement sequence which is substantially complementary to the sequence set forth in SEQ ID NO:25 and capable of forming a duplex therewith. The effector complement sequence which is substantially complementary to the sequence set forth in SEQ ID NO:25 may be the sequence set forth in SEQ ID NO:24. A shmiR in accordance with this example is hereinafter designated “shmiR7”.

In one example, the nucleic acid described herein may comprise a DNA sequence encoding a shmiR comprising: (i) an effector sequence which is substantially complementary to the sequence set forth in SEQ ID NO:26 with the exception of 1, 2, 3 or 4 base mismatches, provided that the effector sequence is capable of forming a duplex with a sequence set forth in SEQ ID NO:26; and (ii) an effector complement sequence comprising a sequence which is substantially complementary to the effector sequence. For example, the shmiR encoded by the nucleic acid may comprise an effector sequence set forth in SEQ ID NO:27 and an effector complement sequence which is substantially complementary to the sequence set forth in SEQ ID NO:27 and capable of forming a duplex therewith. The effector complement sequence which is substantially complementary to the sequence set forth in SEQ ID NO:27 may be the sequence set forth in SEQ ID NO:26. A shmiR in accordance with this example is hereinafter designated “shmiR9”.

In one example, the nucleic acid described herein may comprise a DNA sequence encoding a shmiR comprising: (i) an effector sequence which is substantially complementary to the sequence set forth in SEQ ID NO:28 with the exception of 1, 2, 3 or 4 base mismatches, provided that the effector sequence is capable of forming a duplex with a sequence set forth in SEQ ID NO:28; and (ii) an effector complement sequence comprising a sequence which is substantially complementary to the effector sequence. For example, the shmiR encoded by the nucleic acid may comprise an effector sequence set forth in SEQ ID NO:29 and an effector complement sequence which is substantially complementary to the sequence set forth in SEQ ID NO:29 and capable of forming a duplex therewith. The effector complement sequence which is substantially complementary to the sequence set forth in SEQ ID NO:29 may be the sequence set forth in SEQ ID NO:28. A shmiR in accordance with this example is hereinafter designated “shmiR11”.

In one example, the nucleic acid described herein may comprise a DNA sequence encoding a shmiR comprising: (i) an effector sequence which is substantially complementary to the sequence set forth in SEQ ID NO:30 with the exception of 1, 2, 3 or 4 base mismatches, provided that the effector sequence is capable of forming a duplex with a sequence set forth in SEQ ID NO:30; and (ii) an effector complement sequence comprising a sequence which is substantially complementary to the effector sequence. For example, the shmiR encoded by the nucleic acid may comprise an effector sequence set forth in SEQ ID NO:31 and an effector complement sequence which is substantially complementary to the sequence set forth in SEQ ID NO:31 and capable of forming a duplex therewith. The effector complement sequence which is substantially complementary to the sequence set forth in SEQ ID NO:31 may be the sequence set forth in SEQ ID NO:30. A shmiR in accordance with this example is hereinafter designated “shmiR13”.

In one example, the nucleic acid described herein may comprise a DNA sequence encoding a shmiR comprising: (i) an effector sequence which is substantially complementary to the sequence set forth in SEQ ID NO:32 with the exception of 1, 2, 3 or 4 base mismatches, provided that the effector sequence is capable of forming a duplex with a sequence set forth in SEQ ID NO:32; and (ii) an effector complement sequence comprising a sequence which is substantially complementary to the effector sequence. For example, the shmiR encoded by the nucleic acid may comprise an effector sequence set forth in SEQ ID NO:33 and an effector complement sequence which is substantially complementary to the sequence set forth in SEQ ID NO:33 and capable of forming a duplex therewith. The effector complement sequence which is substantially complementary to the sequence set forth in SEQ ID NO:33 may be the sequence set forth in SEQ ID NO:32. A shmiR in accordance with this example is hereinafter designated “shmiR14”.

In one example, the nucleic acid described herein may comprise a DNA sequence encoding a shmiR comprising: (i) an effector sequence which is substantially complementary to the sequence set forth in SEQ ID NO:34 with the exception of 1, 2, 3 or 4 base mismatches, provided that the effector sequence is capable of forming a duplex with a sequence set forth in SEQ ID NO:34; and (ii) an effector complement sequence comprising a sequence which is substantially complementary to the effector sequence. For example, the shmiR encoded by the nucleic acid may comprise an effector sequence set forth in SEQ ID NO:35 and an effector complement sequence which is substantially complementary to the sequence set forth in SEQ ID NO:35 and capable of forming a duplex therewith. The effector complement sequence which is substantially complementary to the sequence set forth in SEQ ID NO:35 may be the sequence set forth in SEQ ID NO:34. A shmiR in accordance with this example is hereinafter designated “shmiR15”.

In one example, the nucleic acid described herein may comprise a DNA sequence encoding a shmiR comprising: (i) an effector sequence which is substantially complementary to the sequence set forth in SEQ ID NO:36 with the exception of 1, 2, 3 or 4 base mismatches, provided that the effector sequence is capable of forming a duplex with a sequence set forth in SEQ ID NO:36; and (ii) an effector complement sequence comprising a sequence which is substantially complementary to the effector sequence. For example, the shmiR encoded by the nucleic acid may comprise an effector sequence set forth in SEQ ID NO:37 and an effector complement sequence which is substantially complementary to the sequence set forth in SEQ ID NO:37 and capable of forming a duplex therewith. The effector complement sequence which is substantially complementary to the sequence set forth in SEQ ID NO:37 may be the sequence set forth in SEQ ID NO:36. A shmiR in accordance with this example is hereinafter designated “shmiR16”.

In one example, the nucleic acid described herein may comprise a DNA sequence encoding a shmiR comprising: (i) an effector sequence which is substantially complementary to the sequence set forth in SEQ ID NO:38 with the exception of 1, 2, 3 or 4 base mismatches, provided that the effector sequence is capable of forming a duplex with a sequence set forth in SEQ ID NO:38; and (ii) an effector complement sequence comprising a sequence which is substantially complementary to the effector sequence. For example, the shmiR encoded by the nucleic acid may comprise an effector sequence set forth in SEQ ID NO:39 and an effector complement sequence which is substantially complementary to the sequence set forth in SEQ ID NO:39 and capable of forming a duplex therewith. The effector complement sequence which is substantially complementary to the sequence set forth in SEQ ID NO:39 may be the sequence set forth in SEQ ID NO:38. A shmiR in accordance with this example is hereinafter designated “shmiR17”.

In any of the examples described herein, the shmiR encoded by the nucleic acid of the disclosure may comprise, in a 5′ to 3′ direction:

a 5′ flanking sequence of the pri-miRNA backbone;

the effector complement sequence;

the stemloop sequence;

the effector sequence; and

a 3′ flanking sequence of the pri-miRNA backbone.

In any of the examples described herein, the shmiR encoded by the nucleic acid of the disclosure may comprise, in a 5′ to 3′ direction:

a 5′ flanking sequence of the pri-miRNA backbone;

the effector sequence;

the stemloop sequence;

the effector complement sequence; and

a 3′ flanking sequence of the pri-miRNA backbone.

Suitable loop sequences may be selected from those known in the art. However, an exemplary stemloop sequence is set forth in SEQ ID NO: 40.

Suitable primary micro RNA (pri-miRNA or pri-R) backbones for use in a nucleic acid of the disclosure may be selected from those known in the art. For example, the pri-miRNA backbone may be selected from a pri-miR-30a backbone, a pri-miR-155 backbone, a pri-miR-21 backbone and a pri-miR-136 backbone. Preferably, however, the pri-miRNA backbone is a pri-miR-30a backbone. In accordance with an example in which the pri-miRNA backbone is a pri-miR-30a backbone, the 5′ flanking sequence of the pri-miRNA backbone is set forth in SEQ ID NO: 41 and the 3′ flanking sequence of the pri-miRNA backbone is set forth in SEQ ID NO: 42. Thus, the nucleic acid encoding the shmiRs of the disclosure (e.g., shmiR-1 to shmiR-17 described herein) may comprise DNA sequence encoding the sequence set forth in SEQ ID NO: 41 and DNA sequence encoding the sequence set forth in SEQ ID NO: 42.

In one example, the nucleic acid described herein may comprise a DNA sequence selected from the sequence set forth in any one of SEQ ID NOs: 56-68.

In one example, the nucleic acid described herein comprises or consists of a DNA sequence set forth in SEQ ID NO: 56 and encodes a shmiR (shmiR2) comprising or consisting of the sequence set forth in SEQ ID NO: 43.

In one example, the nucleic acid described herein comprises or consists of a DNA sequence set forth in SEQ ID NO: 57 and encodes a shmiR (shmiR3) comprising or consisting of the sequence set forth in SEQ ID NO: 44.

In one example, the nucleic acid described herein comprises or consists of a DNA sequence set forth in SEQ ID NO: 58 and encodes a shmiR (shmiR4) comprising or consisting of the sequence set forth in SEQ ID NO: 45.

In one example, the nucleic acid described herein comprises or consists of a DNA sequence set forth in SEQ ID NO: 59 and encodes a shmiR (shmiR5) comprising or consisting of the sequence set forth in SEQ ID NO: 46.

In one example, the nucleic acid described herein comprises or consists of a DNA sequence set forth in SEQ ID NO: 60 and encodes a shmiR (shmiR6) comprising or consisting of the sequence set forth in SEQ ID NO: 47.

In one example, the nucleic acid described herein comprises or consists of a DNA sequence set forth in SEQ ID NO: 61 and encodes a shmiR (shmiR7) comprising or consisting of the sequence set forth in SEQ ID NO: 48.

In one example, the nucleic acid described herein comprises or consists of a DNA sequence set forth in SEQ ID NO: 62 and encodes a shmiR (shmiR9) comprising or consisting of the sequence set forth in SEQ ID NO: 49.

In one example, the nucleic acid described herein comprises or consists of a DNA sequence set forth in SEQ ID NO: 63 and encodes a shmiR (shmiR11) comprising or consisting of the sequence set forth in SEQ ID NO: 50.

In one example, the nucleic acid described herein comprises or consists of a DNA sequence set forth in SEQ ID NO: 64 and encodes a shmiR (shmiR13) comprising or consisting of the sequence set forth in SEQ ID NO: 51.

In one example, the nucleic acid described herein comprises or consists of a DNA sequence set forth in SEQ ID NO: 65 and encodes a shmiR (shmiR14) comprising or consisting of the sequence set forth in SEQ ID NO: 52.

In one example, the nucleic acid described herein comprises or consists of a DNA sequence set forth in SEQ ID NO: 66 and encodes a shmiR (shmiR15) comprising or consisting of the sequence set forth in SEQ ID NO: 53.

In one example, the nucleic acid described herein comprises or consists of a DNA sequence set forth in SEQ ID NO: 67 and encodes a shmiR (shmiR16) comprising or consisting of the sequence set forth in SEQ ID NO: 54.

In one example, the nucleic acid described herein comprises or consists of a DNA sequence set forth in SEQ ID NO: 68 and encodes a shmiR (shmiR17) comprising or consisting of the sequence set forth in SEQ ID NO: 55.

Exemplary nucleic acids of the disclosure encode a shmiR selected from shmiR2, shmiR3, shmiR5, shmiR9, shmiR13, shmiR14 and shmiR17 as described herein. Nucleic acids of the disclosure encoding shmiRs selected from shmiR3, shmiR13, shmiR14 and shmiR17 as described herein are particularly preferred.

It will be understood by a person of skill in the art that a nucleic acid in accordance with the present disclosure may be combined or used in conjunction with one or more other nucleic acids comprising a DNA sequence encoding a shRNA or shmiR comprising an effector sequence of at least 17 contiguous nucleotides which is substantially complementary to a region of a RNA transcript corresponding to a PABPN1 protein which is causative of OPMD. In one example, a plurality of nucleic acids are provided comprising:

(a) at least one nucleic acid as described herein; and
(b) at least one further nucleic acid selected from:
(i) a nucleic acid comprising a DNA sequence encoding a shmiR as described herein; or
(ii) a nucleic acid comprising a DNA sequence encoding a short hairpin RNA (shRNA) comprising cognate effector and effector complement sequences of a shmiR as described herein;

wherein the shmiR encoded by the nucleic acid at (a) and the shmiR or shRNA encoded by the nucleic acid at (b) comprise different effector sequences.

Accordingly, in one example the plurality of nucleic acids of the disclosure may comprise two or more nucleic acids encoding shmiRs as described herein, such as two, or three, or four, or five, or six, or seven, or eight, or nine, or ten nucleic acids encoding shmiRs as described herein.

In another example, the plurality of nucleic acids of the disclosure comprises at least one nucleic acid encoding a shmiR as described herein and at least one nucleic acid comprising a DNA sequence encoding a shRNA comprising cognate effector and effector complement sequences of a shmiR as described herein. For example, a shRNA comprising the effector sequence and effector complement sequence of shmiR2 is hereinafter designated “shRNA2”. For example, a shRNA comprising the effector sequence and effector complement sequence of shmiR3 is hereinafter designated “shRNA3”. For example, a shRNA comprising the effector sequence and effector complement sequence of shmiR4 is hereinafter designated “shRNA4”. For example, a shRNA comprising the effector sequence and effector complement sequence of shmiR5 is hereinafter designated “shRNA5”. For example, a shRNA comprising the effector sequence and effector complement sequence of shmiR6 is hereinafter designated “shRNA6”. For example, a shRNA comprising the effector sequence and effector complement sequence of shmiR7 is hereinafter designated “shRNA7”. For example, a shRNA comprising the effector sequence and effector complement sequence of shmiR9 is hereinafter designated “shRNA9”. For example, a shRNA comprising the effector sequence and effector complement sequence of shmiR11 is hereinafter designated “shRNA11”. For example, a shRNA comprising the effector sequence and effector complement sequence of shmiR13 is hereinafter designated “shRNA13”. For example, a shRNA comprising the effector sequence and effector complement sequence of shmiR14 is hereinafter designated “shRNA14”. For example, a shRNA comprising the effector sequence and effector complement sequence of shmiR15 is hereinafter designated “shRNA15”. For example, a shRNA comprising the effector sequence and effector complement sequence of shmiR16 is hereinafter designated “shRNA16”. For example, a shRNA comprising the effector sequence and effector complement sequence of shmiR17 is hereinafter designated “shRNA17”.

According to any example in which one or more of the nucleic acid in the plurality of nucleic acids described herein encodes a shRNA, the shRNA may comprise a loop or stem loop sequence positioned between the cognate effector and the effector complement sequences. Suitable loop sequences may be selected from those known in the art. Alternatively, suitable stem loops may be developed de novo. In one example, a nucleic acid of the plurality described herein encoding a shRNA may comprise a DNA sequence encoding a stem loop positioned between the DNA sequences encoding the effector sequence and the effector complement sequence respectively.

In one example, the plurality of nucleic acids described herein comprises a nucleic acid comprising or consisting of a DNA sequence encoding shmiR2, and at least one other nucleic acid of the disclosure which encodes a shmiR or shRNA targeting a region of a PABPN1 mRNA transcript. Exemplary nucleic acids encoding shmiR2 are described herein and shall be taken to apply mutatis mutandis to this example of the disclosure. In one example, the plurality of nucleic acids described herein comprises a nucleic acid which comprises or consists of a DNA sequence set forth in SEQ ID NO: 56 and which encodes a shmiR comprising or consisting of the sequence set forth in SEQ ID NO: 43, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 56 (shmiR2), and (ii) a nucleic acid comprising or consisting of a DNA sequence encoding one of shmiR3-shmiR7, shmiR9, shmiR11 or shmiR13-shmiR17 or the corresponding shRNA of any thereof.

In one example, the plurality of nucleic acids described herein comprises a nucleic acid comprising or consisting of a DNA sequence encoding shmiR3, and at least one other nucleic acid of the disclosure which encodes a shmiR or shRNA targeting a region of a PABPN1 mRNA transcript. Exemplary nucleic acids encoding shmiR3 are described herein and shall be taken to apply mutatis mutandis to this example of the disclosure. In one example, the plurality of nucleic acids described herein comprises a nucleic acid which comprises or consists of a DNA sequence set forth in SEQ ID NO: 57 and which encodes a shmiR comprising or consisting of the sequence set forth in SEQ ID NO: 44, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 57 (shmiR3), and (ii) a nucleic acid comprising or consisting of a DNA sequence encoding one of shmiR2, shmiR4-shmiR7, shmiR9, shmiR11 or shmiR13-shmiR17 or the corresponding shRNA of any thereof.

In one example, the plurality of nucleic acids described herein comprises a nucleic acid comprising or consisting of a DNA sequence encoding shmiR4, and at least one other nucleic acid of the disclosure which encodes a shmiR or shRNA targeting a region of a PABPN1 mRNA transcript. Exemplary nucleic acids encoding shmiR4 are described herein and shall be taken to apply mutatis mutandis to this example of the disclosure. In one example, the plurality of nucleic acids described herein comprises a nucleic acid which comprises or consists of a DNA sequence set forth in SEQ ID NO: 58 and which encodes a shmiR comprising or consisting of the sequence set forth in SEQ ID NO: 45, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 58 (shmiR4), and (ii) a nucleic acid comprising or consisting of a DNA sequence encoding one of shmiR2, shmiR3, shmiR5-shmiR7, shmiR9, shmiR11 or shmiR13-shmiR17 or the corresponding shRNA of any thereof.

In one example, the plurality of nucleic acids described herein comprises a nucleic acid comprising or consisting of a DNA sequence encoding shmiR5, and at least one other nucleic acid of the disclosure which encodes a shmiR or shRNA targeting a region of a PABPN1 mRNA transcript. Exemplary nucleic acids encoding shmiR5 are described herein and shall be taken to apply mutatis mutandis to this example of the disclosure. In one example, the plurality of nucleic acids described herein comprises a nucleic acid which comprises or consists of a DNA sequence set forth in SEQ ID NO: 59 and which encodes a shmiR comprising or consisting of the sequence set forth in SEQ ID NO: 46, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 59 (shmiR5), and (ii) a nucleic acid comprising or consisting of a DNA sequence encoding one of shmiR2-shmiR4, shmiR6-shmiR7, shmiR9, shmiR11 or shmiR13-shmiR17 or the corresponding shRNA of any thereof.

In one example, the plurality of nucleic acids described herein comprises a nucleic acid comprising or consisting of a DNA sequence encoding shmiR6, and at least one other nucleic acid of the disclosure which encodes a shmiR or shRNA targeting a region of a PABPN1 mRNA transcript. Exemplary nucleic acids encoding shmiR6 are described herein and shall be taken to apply mutatis mutandis to this example of the disclosure. In one example, the plurality of nucleic acids described herein comprises a nucleic acid which comprises or consists of a DNA sequence set forth in SEQ ID NO: 60 and which encodes a shmiR comprising or consisting of the sequence set forth in SEQ ID NO: 47, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 60 (shmiR6), and (ii) a nucleic acid comprising or consisting of a DNA sequence encoding one of shmiR2-shmiR5, shmiR7, shmiR9, shmiR11 or shmiR13-shmiR17 or the corresponding shRNA of any thereof.

In one example, the plurality of nucleic acids described herein comprises a nucleic acid comprising or consisting of a DNA sequence encoding shmiR7, and at least one other nucleic acid of the disclosure which encodes a shmiR or shRNA targeting a region of a PABPN1 mRNA transcript. Exemplary nucleic acids encoding shmiR7 are described herein and shall be taken to apply mutatis mutandis to this example of the disclosure. In one example, the plurality of nucleic acids described herein comprises a nucleic acid which comprises or consists of a DNA sequence set forth in SEQ ID NO: 61 and which encodes a shmiR comprising or consisting of the sequence set forth in SEQ ID NO: 48, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 61 (shmiR7), and (ii) a nucleic acid comprising or consisting of a DNA sequence encoding one of shmiR2-shmiR6, shmiR9, shmiR11 or shmiR13-shmiR17 or the corresponding shRNA of any thereof.

In one example, the plurality of nucleic acids described herein comprises a nucleic acid comprising or consisting of a DNA sequence encoding shmiR9, and at least one other nucleic acid of the disclosure which encodes a shmiR or shRNA targeting a region of a PABPN1 mRNA transcript. Exemplary nucleic acids encoding shmiR9 are described herein and shall be taken to apply mutatis mutandis to this example of the disclosure. In one example, the plurality of nucleic acids described herein comprises a nucleic acid which comprises or consists of a DNA sequence set forth in SEQ ID NO: 62 and which encodes a shmiR comprising or consisting of the sequence set forth in SEQ ID NO: 49, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 62 (shmiR9), and (ii) a nucleic acid comprising or consisting of a DNA sequence encoding one of shmiR2-shmiR7, shmiR11 or shmiR13-shmiR17 or the corresponding shRNA of any thereof.

In one example, the plurality of nucleic acids described herein comprises a nucleic acid comprising or consisting of a DNA sequence encoding shmiR11, and at least one other nucleic acid of the disclosure which encodes a shmiR or shRNA targeting a region of a PABPN1 mRNA transcript. Exemplary nucleic acids encoding shmiR11 are described herein and shall be taken to apply mutatis mutandis to this example of the disclosure. In one example, the plurality of nucleic acids described herein comprises a nucleic acid which comprises or consists of a DNA sequence set forth in SEQ ID NO: 63 and which encodes a shmiR comprising or consisting of the sequence set forth in SEQ ID NO: 50, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 63 (shmiR11), and (ii) a nucleic acid comprising or consisting of a DNA sequence encoding one of shmiR2-shmiR7, shmiR9 or shmiR13-shmiR17 or the corresponding shRNA of any thereof.

In one example, the plurality of nucleic acids described herein comprises a nucleic acid comprising or consisting of a DNA sequence encoding shmiR13, and at least one other nucleic acid of the disclosure which encodes a shmiR or shRNA targeting a region of a PABPN1 mRNA transcript. Exemplary nucleic acids encoding shmiR13 are described herein and shall be taken to apply mutatis mutandis to this example of the disclosure. In one example, the plurality of nucleic acids described herein comprises a nucleic acid which comprises or consists of a DNA sequence set forth in SEQ ID NO: 64 and which encodes a shmiR comprising or consisting of the sequence set forth in SEQ ID NO: 51, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 64 (shmiR13), and (ii) a nucleic acid comprising or consisting of a DNA sequence encoding one of shmiR2-shmiR7, shmiR9, shmiR11 or shmiR14-shmiR17 or the corresponding shRNA of any thereof.

In one example, the plurality of nucleic acids described herein comprises a nucleic acid comprising or consisting of a DNA sequence encoding shmiR14, and at least one other nucleic acid of the disclosure which encodes a shmiR or shRNA targeting a region of a PABPN1 mRNA transcript. Exemplary nucleic acids encoding shmiR14 are described herein and shall be taken to apply mutatis mutandis to this example of the disclosure. In one example, the plurality of nucleic acids described herein comprises a nucleic acid which comprises or consists of a DNA sequence set forth in SEQ ID NO: 65 and which encodes a shmiR comprising or consisting of the sequence set forth in SEQ ID NO: 52, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 65 (shmiR14), and (ii) a nucleic acid comprising or consisting of a DNA sequence encoding one of shmiR2-shmiR7, shmiR9, shmiR11 or shmiR13, shmiR15-shmiR17 or the corresponding shRNA of any thereof.

In one example, the plurality of nucleic acids described herein comprises a nucleic acid comprising or consisting of a DNA sequence encoding shmiR15, and at least one other nucleic acid of the disclosure which encodes a shmiR or shRNA targeting a region of a PABPN1 mRNA transcript. Exemplary nucleic acids encoding shmiR15 are described herein and shall be taken to apply mutatis mutandis to this example of the disclosure. In one example, the plurality of nucleic acids described herein comprises a nucleic acid which comprises or consists of a DNA sequence set forth in SEQ ID NO: 66 and which encodes a shmiR comprising or consisting of the sequence set forth in SEQ ID NO: 53, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 66 (shmiR15), and (ii) a nucleic acid comprising or consisting of a DNA sequence encoding one of shmiR2-shmiR7, shmiR9, shmiR11 or shmiR13-shmiR14, or shmiR16-shmiR17 or the corresponding shRNA of any thereof.

In one example, the plurality of nucleic acids described herein comprises a nucleic acid comprising or consisting of a DNA sequence encoding shmiR16, and at least one other nucleic acid of the disclosure which encodes a shmiR or shRNA targeting a region of a PABPN1 mRNA transcript. Exemplary nucleic acids encoding shmiR16 are described herein and shall be taken to apply mutatis mutandis to this example of the disclosure. In one example, the plurality of nucleic acids described herein comprises a nucleic acid which comprises or consists of a DNA sequence set forth in SEQ ID NO: 67 and which encodes a shmiR comprising or consisting of the sequence set forth in SEQ ID NO: 54, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 67 (shmiR16), and (ii) a nucleic acid comprising or consisting of a DNA sequence encoding one of shmiR2-shmiR7, shmiR9, shmiR11 or shmiR13-shmiR15, or shmiR17 or the corresponding shRNA of any thereof.

In one example, the plurality of nucleic acids described herein comprises a nucleic acid comprising or consisting of a DNA sequence encoding shmiR17, and at least one other nucleic acid of the disclosure which encodes a shmiR or shRNA targeting a region of a PABPN1 mRNA transcript. Exemplary nucleic acids encoding shmiR17 are described herein and shall be taken to apply mutatis mutandis to this example of the disclosure. In one example, the plurality of nucleic acids described herein comprises a nucleic acid which comprises or consists of a DNA sequence set forth in SEQ ID NO: 68 and which encodes a shmiR comprising or consisting of the sequence set forth in SEQ ID NO: 55, and at least one other nucleic acid of the disclosure which encodes a shmiR or shRNA targeting a region of a PABPN1 mRNA transcript. For example, the plurality of nucleic acids described herein may comprise (i) a nucleic acid comprising or consisting of a DNA sequence set forth in SEQ ID NO: 68 (shmiR17), and (ii) a nucleic acid comprising or consisting of a DNA sequence encoding one of shmiR2-shmiR7, shmiR9, shmiR11 or shmiR13-shmiR16 or the corresponding shRNA of any thereof.

In accordance with any example of a plurality of nucleic acids as described herein, the plurality of nucleic acids may comprise two or more nucleic acids encoding shmiRs or shRNAs as described herein, such as two, or three, or four, or five, or six, or seven, or eight, or nine, or ten nucleic acids encoding shmiRs as described herein, provided at that at least one of the nucleic acids encodes a shmiRs of the disclosure.

In one example, the plurality of nucleic acids comprises two nucleic acids encoding a shmiR or shRNA described herein, with the proviso that at least one of the nucleic acids encodes a shmiR as described herein. In one example, the plurality of nucleic acids comprises three nucleic acids encoding a shmiR or shRNA described herein, with the proviso that at least one of the nucleic acids encodes a shmiR as described herein. In one example, the plurality of nucleic acids comprises four nucleic acids encoding a shmiR or shRNA described herein, with the proviso that at least one of the nucleic acids encodes a shmiR as described herein. In one example, the plurality of nucleic acids comprises five nucleic acids encoding a shmiR or shRNA described herein, with the proviso that at least one of the nucleic acids encodes a shmiR as described herein. In one example, the plurality of nucleic acids comprises six nucleic acids encoding a shmiR or shRNA described herein, with the proviso that at least one of the nucleic acids encodes a shmiR as described herein. In one example, the plurality of nucleic acids comprises seven nucleic acids encoding a shmiR or shRNA described herein, with the proviso that at least one of the nucleic acids encodes a shmiR as described herein. In one example, the plurality of nucleic acids comprises eight nucleic acids encoding a shmiR or shRNA described herein, with the proviso that at least one of the nucleic acids encodes a shmiR as described herein. In one example, the plurality of nucleic acids comprises nine nucleic acids encoding a shmiR or shRNA described herein, with the proviso that at least one of the nucleic acids encodes a shmiR as described herein. In one example, the plurality of nucleic acids comprises ten nucleic acids encoding a shmiR or shRNA described herein, with the proviso that at least one of the nucleic acids encodes a shmiR as described herein.

In one example of a plurality of nucleic acids described herein, one of the nucleic acids comprises a DNA sequence encoding a shmiR having an effector sequence which is substantially complementary to a region of corresponding length in an RNA transcript comprising or consisting of the sequence set forth in SEQ ID NO: 1. Suitable nucleic acids encoding a shmiR having an effector sequence which is substantially complementary to a region of corresponding length in an RNA transcript comprising or consisting of the sequence set forth in SEQ ID NO: 1 are described herein e.g., for shmiR2.

In one example of a plurality of nucleic acids described herein, one of the nucleic acids comprises a DNA sequence encoding a shmiR having an effector sequence which is substantially complementary to a region of corresponding length in an RNA transcript comprising or consisting of the sequence set forth in SEQ ID NO: 2. Suitable nucleic acids encoding a shmiR having an effector sequence which is substantially complementary to a region of corresponding length in an RNA transcript comprising or consisting of the sequence set forth in SEQ ID NO: 2 are described herein e.g., for shmiR3.

In one example of a plurality of nucleic acids described herein, one of the nucleic acids comprises a DNA sequence encoding a shmiR having an effector sequence which is substantially complementary to a region of corresponding length in an RNA transcript comprising or consisting of the sequence set forth in SEQ ID NO: 4. Suitable nucleic acids encoding a shmiR having an effector sequence which is substantially complementary to a region of corresponding length in an RNA transcript comprising or consisting of the sequence set forth in SEQ ID NO: 4 are described herein e.g., for shmiR5.

In one example of a plurality of nucleic acids described herein, one of the nucleic acids comprises a DNA sequence encoding a shmiR having an effector sequence which is substantially complementary to a region of corresponding length in an RNA transcript comprising or consisting of the sequence set forth in SEQ ID NO: 7. Suitable nucleic acids encoding a shmiR having an effector sequence which is substantially complementary to a region of corresponding length in an RNA transcript comprising or consisting of the sequence set forth in SEQ ID NO: 7 are described herein e.g., for shmiR9.

In one example of a plurality of nucleic acids described herein, one of the nucleic acids comprises a DNA sequence encoding a shmiR having an effector sequence which is substantially complementary to a region of corresponding length in an RNA transcript comprising or consisting of the sequence set forth in SEQ ID NO: 9. Suitable nucleic acids encoding a shmiR having an effector sequence which is substantially complementary to a region of corresponding length in an RNA transcript comprising or consisting of the sequence set forth in SEQ ID NO: 9 are described herein e.g., for shmiR13.

In one example of a plurality of nucleic acids described herein, one of the nucleic acids comprises a DNA sequence encoding a shmiR having an effector sequence which is substantially complementary to a region of corresponding length in an RNA transcript comprising or consisting of the sequence set forth in SEQ ID NO: 10. Suitable nucleic acids encoding a shmiR having an effector sequence which is substantially complementary to a region of corresponding length in an RNA transcript comprising or consisting of the sequence set forth in SEQ ID NO: 10 are described herein e.g., for shmiR14.

In one example of a plurality of nucleic acids described herein, one of the nucleic acids comprises a DNA sequence encoding a shmiR having an effector sequence which is substantially complementary to a region of corresponding length in an RNA transcript comprising or consisting of the sequence set forth in SEQ ID NO: 13. Suitable nucleic acids encoding a shmiR having an effector sequence which is substantially complementary to a region of corresponding length in an RNA transcript comprising or consisting of the sequence set forth in SEQ ID NO: 13 are described herein e.g., for shmiR17.

An exemplary plurality of nucleic acids of the disclosure comprises at least two nucleic acids, each comprising a DNA sequence encoding a shmiR of the disclosure, wherein each shmiR comprises a different effector sequence.

In one example, each of the at least two nucleic acids encode a shmiR comprising an effector sequence which is substantially complementary to a region of corresponding length in an RNA transcript set forth in one of SEQ ID NOs: 1, 2, 4, 7, 9, 10 and 13. Exemplary nucleic acids of the disclosure encoding shmiRs comprising effector sequences which are substantially complementary to regions of corresponding length in the RNA transcripts set forth in SEQ ID NO: 1, 2, 4, 7, 9, 10 and 13 are described herein and shall be taken to apply mutatis mutandis to this example of the disclosure.

In one example, the at least two nucleic acids are selected from the group consisting of:

a nucleic acid comprising or consisting of a DNA sequence encoding a shmiR comprising an effector sequence set forth in SEQ ID NO: 15 and an effector complement sequence set forth in SEQ ID NO: 14 e.g., a nucleic acid comprising or consisting of a DNA sequence set forth in SEQ ID NO: 56 (shmiR2);

a nucleic acid comprising or consisting of a DNA sequence encoding a shmiR comprising an effector sequence set forth in SEQ ID NO: 17 and an effector complement sequence set forth in SEQ ID NO: 16 e.g., a nucleic acid comprising or consisting of a DNA sequence set forth in SEQ ID NO: 57 (shmiR3);

a nucleic acid comprising or consisting of a DNA sequence encoding a shmiR comprising an effector sequence set forth in SEQ ID NO: 21 and an effector complement sequence set forth in SEQ ID NO: 20 e.g., a nucleic acid comprising or consisting of a DNA sequence set forth in SEQ ID NO: 59 (shmiR5);

a nucleic acid comprising or consisting of a DNA sequence encoding a shmiR comprising an effector sequence set forth in SEQ ID NO: 27 and an effector complement sequence set forth in SEQ ID NO: 26 e.g., a nucleic acid comprising or consisting of a DNA sequence set forth in SEQ ID NO: 62 (shmiR9);

a nucleic acid comprising or consisting of a DNA sequence encoding a shmiR comprising an effector sequence set forth in SEQ ID NO: 31 and an effector complement sequence set forth in SEQ ID NO: 30 e.g., a nucleic acid comprising or consisting of a DNA sequence set forth in SEQ ID NO: 64 (shmiR13);

a nucleic acid comprising or consisting of a DNA sequence encoding a shmiR comprising an effector sequence set forth in SEQ ID NO: 33 and an effector complement sequence set forth in SEQ ID NO: 32 e.g., a nucleic acid comprising or consisting of a DNA sequence set forth in SEQ ID NO: 65 (shmiR14); and

a nucleic acid comprising or consisting of a DNA sequence encoding a shmiR comprising an effector sequence set forth in SEQ ID NO: 39 and an effector complement sequence set forth in SEQ ID NO: 38 e.g., a nucleic acid comprising or consisting of a DNA sequence set forth in SEQ ID NO: 68 (shmiR17).

In one example, each of the at least two nucleic acids encode a shmiR comprising an effector sequence which is substantially complementary to a region of corresponding length in an RNA transcript set forth in one of SEQ ID NOs: 2, 9, 10 and 13. Exemplary nucleic acids of the disclosure encoding shmiRs comprising effector sequences which are substantially complementary to regions of corresponding length in the RNA transcripts set forth in SEQ ID NO: 2, 9, 10 and 13 are described herein and shall be taken to apply mutatis mutandis to this example of the disclosure.

In one example, the at least two nucleic acids are selected from the group consisting of:

a nucleic acid comprising or consisting of a DNA sequence encoding a shmiR comprising an effector sequence set forth in SEQ ID NO: 17 and an effector complement sequence set forth in SEQ ID NO: 16 e.g., a nucleic acid comprising or consisting of a DNA sequence set forth in SEQ ID NO: 57 (shmiR3);

a nucleic acid comprising or consisting of a DNA sequence encoding a shmiR comprising an effector sequence set forth in SEQ ID NO: 31 and an effector complement sequence set forth in SEQ ID NO: 30 e.g., a nucleic acid comprising or consisting of a DNA sequence set forth in SEQ ID NO: 64 (shmiR13);

a nucleic acid comprising or consisting of a DNA sequence encoding a shmiR comprising an effector sequence set forth in SEQ ID NO: 33 and an effector complement sequence set forth in SEQ ID NO: 32 e.g., a nucleic acid comprising or consisting of a DNA sequence set forth in SEQ ID NO: 65 (shmiR14); and

a nucleic acid comprising or consisting of a DNA sequence encoding a shmiR comprising an effector sequence set forth in SEQ ID NO: 39 and an effector complement sequence set forth in SEQ ID NO: 38 e.g., a nucleic acid comprising or consisting of a DNA sequence set forth in SEQ ID NO: 68 (shmiR17).

In one example, the plurality of nucleic acids comprises a nucleic acid encoding a shmiR comprising an effector sequence which is substantially complementary to a region of corresponding length in an RNA transcript set forth in SEQ ID NO: 9, and a nucleic acid encoding a shmiR comprising an effector sequence which is substantially complementary to a region of corresponding length in an RNA transcript set forth in SEQ ID NO: 13. For example, the plurality of nucleic acids may comprise:

(a) a nucleic acid comprising or consisting of a DNA sequence encoding a shmiR comprising an effector sequence set forth in SEQ ID NO: 31 and an effector complement sequence set forth in SEQ ID NO: 30 e.g., a nucleic acid comprising or consisting of a DNA sequence set forth in SEQ ID NO: 64 (shmiR13); and
(b) a nucleic acid comprising or consisting of a DNA sequence encoding a shmiR comprising an effector sequence set forth in SEQ ID NO: 39 and an effector complement sequence set forth in SEQ ID NO: 38 e.g., a nucleic acid comprising or consisting of a DNA sequence set forth in SEQ ID NO: 68 (shmiR17).

An exemplary plurality of nucleic acids of the disclosure comprises a nucleic acid comprising or consisting of a DNA sequence set forth in SEQ ID NO: 64 (shmiR13) and a nucleic acid comprising or consisting of a DNA sequence set forth in SEQ ID NO: 68 (shmiR17).

In one example, the plurality of nucleic acids comprises a nucleic acid encoding a shmiR comprising an effector sequence which is substantially complementary to a region of corresponding length in an RNA transcript set forth in SEQ ID NO: 2, and a nucleic acid encoding a shmiR comprising an effector sequence which is substantially complementary to a region of corresponding length in an RNA transcript set forth in SEQ ID NO: 10. For example, the plurality of nucleic acids may comprise:

(a) a nucleic acid comprising or consisting of a DNA sequence encoding a shmiR comprising an effector sequence set forth in SEQ ID NO: 17 and an effector complement sequence set forth in SEQ ID NO: 16, e.g., a nucleic acid comprising or consisting of a DNA sequence set forth in SEQ ID NO: 57 (shmiR3); and
(b) a nucleic acid comprising or consisting of a DNA sequence encoding a shmiR comprising an effector sequence set forth in SEQ ID NO: 33 and an effector complement sequence set forth in SEQ ID NO: 32 e.g., a nucleic acid comprising or consisting of the sequence set forth in SEQ ID NO:65 (shmiR14).

An exemplary plurality of nucleic acids of the disclosure comprises a nucleic acid comprising or consisting of a DNA sequence set forth in SEQ ID NO: 57 (shmiR3) and a nucleic acid comprising or consisting of a DNA sequence set forth in SEQ ID NO: 65 (shmiR14).

In accordance with an example in which a plurality of nucleic acids is provided, two or more of the nucleic acids may form separate parts of the same polynucleotide. In another example, two or more of the nucleic acids in the plurality form parts of different polynucleotides, respectively. In another example, the plurality of nucleic acids described herein are provided as multiple components e.g., multiple compositions. For example, each of the nucleic acids of the plurality may be provided separately. Alternatively, in an example where three or more nucleic acids of the disclosure are provided, at least one of the nucleic acids may be provided separately and two or more of the plurality provided together.

In some examples, the or each nucleic acid in accordance with the present disclosure may comprise, or be in operable linkage with, additional elements e.g., to facilitate transcription of the shmiR or shRNA. For example, the or each nucleic acid may comprise a promoter operably linked to the sequence encoding a shmiR or shRNA described herein. Other elements e.g., transcriptional terminators and initiators, are known in the art and/or described herein.

Alternatively, or in addition, the or each nucleic acid in accordance with the present disclosure may comprise one or more restriction sites e.g., to facilitate cloning of the nucleic acid(s) into cloning or expression vectors. For example, the nucleic acids described herein may include a restriction site upstream and/or downstream of the sequence encoding a shmiR or shRNA of the disclosure. Suitable restriction enzyme recognition sequences will be known to a person of skill in the art. However, in one example, the nucleic acid(s) of the disclosure may include a BamH1 restriction site (GGATCC) at the 5′ terminus i.e., upstream of the sequence encoding the shmiR or shRNA, and a EcoR1 restriction site (GAATTC) at the 3′ terminus i.e., downstream of the sequence encoding the shmiR or shRNA.

ddRNAi Constructs

In one example, the or each nucleic acid of the disclosure is provided in the form of, or is comprised in, a DNA-directed RNAi (ddRNAi) construct. Accordingly, in one example, the present disclosure provides a ddRNAi construct comprising a nucleic acid as described herein. In another example, the present disclosure provides a ddRNAi construct comprising a plurality of nucleic acids described herein. In yet another example, the present disclosure provides a plurality of ddRNAi constructs, each comprising a nucleic acid of the plurality of nucleic acids as described herein (i.e., such that all of the nucleic acids of the plurality are represented in the plurality of ddRNAi constructs). Exemplary nucleic acids encoding shmiRs or shRNAs comprising effector sequences targeting a mRNA transcript of PABPN1 which is causative of OPMD are described herein and shall be taken to apply mutatis mutandis to this example of the disclosure.

In one example, the ddRNAi construct comprises a nucleic acid of the disclosure operably linked to a promoter.

In accordance with an example in which the ddRNAi construct comprises a plurality of the nucleic acids described herein, each of the nucleic acids may be operably-linked to a promoter. In one example, the nucleic acids in the ddRNAi construct may be operably linked to the same promoter. In one example, the nucleic acids in the ddRNAi construct may be operably linked to different promoters.

In one example, a ddRNAi construct of the disclosure comprises a nucleic acid comprising or consisting of a DNA sequence encoding shmiR2. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 1. Exemplary nucleic acids encoding shmiR2 are described herein and shall be taken to apply mutatis mutandis to this example of the disclosure. In one example, the ddRNAi construct comprises a nucleic acid which comprises or consists of a DNA sequence set forth in SEQ ID NO: 56 and which encodes a shmiR comprising or consisting of the sequence set forth in SEQ ID NO: 43. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR3-shmiR7, shmiR9, shmiR11 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 56 (shmiR2), and (ii) a nucleic acid comprising or consisting of a DNA sequence encoding one of shmiR3-shmiR7, shmiR9, shmiR11 or shmiR13-shmiR17 or the corresponding shRNA of any thereof. Exemplary nucleic acids encoding shmiRs designated shmiR3-shmiR7, shmiR9, shmiR11 and shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR3. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 2. Exemplary nucleic acids encoding shmiR3 are described herein and shall be taken to apply mutatis mutandis to this example of the disclosure. In one example, the ddRNAi construct comprises a nucleic acid which comprises or consists of a DNA sequence set forth in SEQ ID NO: 57 and which encodes a shmiR comprising or consisting of the sequence set forth in SEQ ID NO: 44. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2, shmiR4-shmiR7, shmiR9, shmiR11 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 57 (shmiR3), and (ii) a nucleic acid comprising or consisting of a DNA sequence encoding one of shmiR2, shmiR4-shmiR7, shmiR9, shmiR11 or shmiR13-shmiR17 or the corresponding shRNA of any thereof. Exemplary nucleic acids encoding shmiRs designated shmiR2, shmiR4-shmiR7, shmiR9, shmiR11 or shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR4. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 3. Exemplary nucleic acids encoding shmiR4 are described herein and shall be taken to apply mutatis mutandis to this example of the disclosure. In one example, the ddRNAi construct comprises a nucleic acid which comprises or consists of a DNA sequence set forth in SEQ ID NO: 58 and which encodes a shmiR comprising or consisting of the sequence set forth in SEQ ID NO: 45. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2, shmiR3, shmiR5-shmiR7, shmiR9, shmiR11 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 58 (shmiR4), and (ii) a nucleic acid comprising or consisting of a DNA sequence encoding one of shmiR2, shmiR3, shmiR5-shmiR7, shmiR9, shmiR11 or shmiR13-shmiR17 or the corresponding shRNA of any thereof. Exemplary nucleic acids encoding shmiRs designated shmiR2, shmiR3, shmiR5-shmiR7, shmiR9, shmiR11 or shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR5. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 4. Exemplary nucleic acids encoding shmiR5 are described herein and shall be taken to apply mutatis mutandis to this example of the disclosure. In one example, the ddRNAi construct comprises a nucleic acid which comprises or consists of a DNA sequence set forth in SEQ ID NO: 59 and which encodes a shmiR comprising or consisting of the sequence set forth in SEQ ID NO: 46. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR4, shmiR6-shmiR7, shmiR9, shmiR11 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 59 (shmiR5), and (ii) a nucleic acid comprising or consisting of a DNA sequence encoding one of shmiR2-shmiR4, shmiR6-shmiR7, shmiR9, shmiR11 or shmiR13-shmiR17 or the corresponding shRNA of any thereof. Exemplary nucleic acids encoding shmiRs designated shmiR2-shmiR4, shmiR6-shmiR7, shmiR9, shmiR11 or shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR6. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 5. Exemplary nucleic acids encoding shmiR6 are described herein and shall be taken to apply mutatis mutandis to this example of the disclosure. In one example, the ddRNAi construct comprises a nucleic acid which comprises or consists of a DNA sequence set forth in SEQ ID NO: 60 and which encodes a shmiR comprising or consisting of the sequence set forth in SEQ ID NO: 47. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of s shmiR2-shmiR5, shmiR7, shmiR9, shmiR11 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 60 (shmiR6), and (ii) a nucleic acid comprising or consisting of a DNA sequence encoding one of shmiR2-shmiR5, shmiR7, shmiR9, shmiR11 or shmiR13-shmiR17 or the corresponding shRNA of any thereof. Exemplary nucleic acids encoding shmiRs designated shmiR2-shmiR5, shmiR7, shmiR9, shmiR11 or shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR7. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 6. Exemplary nucleic acids encoding shmiR7 are described herein and shall be taken to apply mutatis mutandis to this example of the disclosure. In one example, the ddRNAi construct comprises a nucleic acid which comprises or consists of a DNA sequence set forth in SEQ ID NO: 61 and which encodes a shmiR comprising or consisting of the sequence set forth in SEQ ID NO: 48. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR6, shmiR9, shmiR11 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 61 (shmiR7), and (ii) a nucleic acid comprising or consisting of a DNA sequence encoding one of shmiR2-shmiR6, shmiR9, shmiR11 or shmiR13-shmiR17 or the corresponding shRNA of any thereof. Exemplary nucleic acids encoding shmiRs designated shmiR2-shmiR6, shmiR9, shmiR11 or shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR9. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 7. Exemplary nucleic acids encoding shmiR9 are described herein and shall be taken to apply mutatis mutandis to this example of the disclosure. In one example, the ddRNAi construct comprises a nucleic acid which comprises or consists of a DNA sequence set forth in SEQ ID NO: 62 and which encodes a shmiR comprising or consisting of the sequence set forth in SEQ ID NO: 49. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR7, shmiR11 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 62 (shmiR9), and (ii) a nucleic acid comprising or consisting of a DNA sequence encoding one of shmiR2-shmiR7, shmiR11 or shmiR13-shmiR17 or the corresponding shRNA of any thereof. Exemplary nucleic acids encoding shmiRs designated shmiR2-shmiR7, shmiR11 or shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR11. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 8. Exemplary nucleic acids encoding shmiR11 are described herein and shall be taken to apply mutatis mutandis to this example of the disclosure. In one example, the ddRNAi construct comprises a nucleic acid which comprises or consists of a DNA sequence set forth in SEQ ID NO: 63 and which encodes a shmiR comprising or consisting of the sequence set forth in SEQ ID NO: 50. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR7, shmiR9 or shmiR13-shmiR17 or the corresponding shRNA of any thereof, as described herein. For example, the ddRNAi construct described herein may comprise (i) a nucleic acid comprising or consisting of a DNA sequence set forth in SEQ ID NO: 63 (shmiR11), and (ii) a nucleic acid comprising or consisting of a DNA sequence encoding one of shmiR2-shmiR7, shmiR9 or shmiR13-shmiR17 or the corresponding shRNA of any thereof. Exemplary nucleic acids encoding shmiRs designated shmiR2-shmiR7, shmiR9 or shmiR13-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR13. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 9. Exemplary nucleic acids encoding shmiR13 are described herein and shall be taken to apply mutatis mutandis to this example of the disclosure. In one example, the ddRNAi construct comprises a nucleic acid which comprises or consists of a DNA sequence set forth in SEQ ID NO: 64 and which encodes a shmiR comprising or consisting of the sequence set forth in SEQ ID NO: 51. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR7, shmiR9, shmiR11 or shmiR14-shmiR17 or the corresponding shRNA of any thereof, as described herein. For example, the ddRNAi construct described herein may comprise (i) a nucleic acid comprising or consisting of a DNA sequence set forth in SEQ ID NO: 64 (shmiR13), and (ii) a nucleic acid comprising or consisting of a DNA sequence encoding one of shmiR2-shmiR7, shmiR9, shmiR11 or shmiR14-shmiR17 or the corresponding shRNA of any thereof. Exemplary nucleic acids encoding shmiRs designated shmiR2-shmiR7, shmiR9, shmiR11 or shmiR14-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR14. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 10. Exemplary nucleic acids encoding shmiR14 are described herein and shall be taken to apply mutatis mutandis to this example of the disclosure. In one example, the ddRNAi construct comprises a nucleic acid which comprises or consists of a DNA sequence set forth in SEQ ID NO: 65 and which encodes a shmiR comprising or consisting of the sequence set forth in SEQ ID NO: 52. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR7, shmiR9, shmiR11 or shmiR13, shmiR15-shmiR17 or the corresponding shRNA of any thereof, as described herein. For example, the ddRNAi construct described herein may comprise (i) a nucleic acid comprising or consisting of a DNA sequence set forth in SEQ ID NO: 65 (shmiR14), and (ii) a nucleic acid comprising or consisting of a DNA sequence encoding one of shmiR2-shmiR7, shmiR9, shmiR11 or shmiR13, shmiR15-shmiR17 or the corresponding shRNA of any thereof. Exemplary nucleic acids encoding shmiRs designated shmiR2-shmiR7, shmiR9, shmiR11 or shmiR13, shmiR15-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR15. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 11. Exemplary nucleic acids encoding shmiR15 are described herein and shall be taken to apply mutatis mutandis to this example of the disclosure. In one example, the ddRNAi construct comprises a nucleic acid which comprises or consists of a DNA sequence set forth in SEQ ID NO: 66 and which encodes a shmiR comprising or consisting of the sequence set forth in SEQ ID NO: 53. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR7, shmiR9, shmiR11 or shmiR13-shmiR14, or shmiR16-shmiR17 or the corresponding shRNA of any thereof, as described herein. For example, the ddRNAi construct described herein may comprise (i) a nucleic acid comprising or consisting of a DNA sequence set forth in SEQ ID NO: 66 (shmiR15), and (ii) a nucleic acid comprising or consisting of a DNA sequence encoding one of shmiR2-shmiR7, shmiR9, shmiR11 or shmiR13-shmiR14, or shmiR16-shmiR17 or the corresponding shRNA of any thereof. Exemplary nucleic acids encoding shmiRs designated shmiR2-shmiR7, shmiR9, shmiR11 or shmiR13-shmiR14, or shmiR16-shmiR17 are described herein and shall be taken to apply mutatis mutandis to this example of the disclosure.

In one example, a ddRNAi construct of the disclosure comprises a nucleic acid comprising or consisting of a DNA sequence encoding shmiR16. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 12. Exemplary nucleic acids encoding shmiR16 are described herein and shall be taken to apply mutatis mutandis to this example of the disclosure. In one example, the ddRNAi construct comprises a nucleic acid which comprises or consists of a DNA sequence set forth in SEQ ID NO: 67 and which encodes a shmiR comprising or consisting of the sequence set forth in SEQ ID NO: 54. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR7, shmiR9, shmiR11 or shmiR13-shmiR15, or shmiR17 or the corresponding shRNA of any thereof, as described herein. For example, the ddRNAi construct described herein may comprise (i) a nucleic acid comprising or consisting of a DNA sequence set forth in SEQ ID NO: 67 (shmiR16), and (ii) a nucleic acid comprising or consisting of a DNA sequence encoding one of shmiR2-shmiR7, shmiR9, shmiR11 or shmiR13-shmiR15, or shmiR17 or the corresponding shRNA of any thereof. Exemplary nucleic acids encoding shmiRs designated shmiR2-shmiR7, shmiR9, shmiR11 or shmiR13-shmiR15, or shmiR17 are described herein and shall be taken to apply mutatis mutandis to this example of the disclosure.

In one example, a ddRNAi construct of the disclosure comprises a nucleic acid comprising or consisting of a DNA sequence encoding shmiR17. For example, the ddRNAi construct may comprise a nucleic acid comprising or consisting of a DNA sequence encoding a shmiR having an effector sequence which is substantially complementary to a region of corresponding length in an RNA transcript comprising or consisting of the sequence set forth in SEQ ID NO: 13. Exemplary nucleic acids encoding shmiR17 are described herein and shall be taken to apply mutatis mutandis to this example of the disclosure. In one example, the ddRNAi construct comprises a nucleic acid which comprises or consists of a DNA sequence set forth in SEQ ID NO: 68 and which encodes a shmiR comprising or consisting of the sequence set forth in SEQ ID NO: 55. The ddRNAi construct may comprise one or more further nucleic acids of the disclosure comprising a DNA sequence encoding a shmiR or shRNA targeting a region of a PABPN1 mRNA transcript, such as a nucleic acid comprising or consisting of a DNA sequence encoding one of shmiR2-shmiR7, shmiR9, shmiR11 or shmiR13-shmiR16 or the corresponding shRNA of any thereof, as described herein. For example, the ddRNAi construct described herein may comprise (i) a nucleic acid comprising or consisting of a DNA sequence set forth in SEQ ID NO: 68 (shmiR17), and (ii) a nucleic acid comprising or consisting of a DNA sequence encoding one of shmiR2-shmiR7, shmiR9, shmiR11 or shmiR13-shmiR16 or the corresponding shRNA of any thereof. Exemplary nucleic acids encoding shmiRs designated shmiR2-shmiR7, shmiR9, shmiR11 or shmiR13-shmiR16 are described herein and shall be taken to apply mutatis mutandis to this example of the disclosure.

In accordance with any example of a ddRNAi construct comprising a plurality of nucleic acids as described herein, the ddRNAi construct may comprise two or more nucleic acids encoding shmiRs or shRNAs as described herein, such as two, or three, or four, or five, or six, or seven, or eight, or nine, or ten nucleic acids encoding shmiRs or shRNAs as described herein, provided that at least one of the nucleic acids encodes a shmiR as described herein.

In one example, the ddRNAi construct comprises two nucleic acids encoding a shmiR or shRNA described herein, with the proviso that at least one of the nucleic acids encodes a shmiR as described herein. In one example, the ddRNAi construct comprises three nucleic acids encoding a shmiR or shRNA described herein, with the proviso that at least one of the nucleic acids encodes a shmiR as described herein. In one example, the ddRNAi construct comprises four nucleic acids encoding a shmiR or shRNA described herein, with the proviso that at least one of the nucleic acids encodes a shmiR as described herein. In one example, the ddRNAi construct comprises five nucleic acids encoding a shmiR or shRNA described herein, with the proviso that at least one of the nucleic acids encodes a shmiR as described herein. In one example, the ddRNAi construct comprises six nucleic acids encoding a shmiR or shRNA described herein, with the proviso that at least one of the nucleic acids encodes a shmiR as described herein. In one example, the ddRNAi construct comprises seven nucleic acids encoding a shmiR or shRNA described herein, with the proviso that at least one of the nucleic acids encodes a shmiR as described herein. In one example, the ddRNAi construct comprises eight nucleic acids encoding a shmiR or shRNA described herein, with the proviso that at least one of the nucleic acids encodes a shmiR as described herein. In one example, the ddRNAi construct comprises nine nucleic acids encoding a shmiR or shRNA described herein, with the proviso that at least one of the nucleic acids encodes a shmiR as described herein. In one example, the ddRNAi construct comprises ten nucleic acids encoding a shmiR or shRNA described herein, with the proviso that at least one of the nucleic acids encodes a shmiR as described herein.

An exemplary ddRNAi construct of the disclosure comprises at least two nucleic acids, each comprising a DNA sequence encoding a shmiR of the disclosure, wherein each shmiR comprises a different effector sequence. In one example, each of the at least two nucleic acids in the ddRNAi construct encode a shmiR comprising an effector sequence which is substantially complementary to a region of corresponding length in an RNA transcript set forth in one of SEQ ID NOs: 1, 2, 4, 7, 9, 10 and 13. Exemplary nucleic acids of the disclosure encoding shmiRs comprising effector sequences which are substantially complementary to regions of corresponding length in the RNA transcripts set forth in SEQ ID NO: 1, 2, 4, 7, 9, 10 and 13 are described herein and shall be taken to apply mutatis mutandis to this example of the disclosure describing ddRNAi constructs.

In one example, the ddRNAi construct comprises at least two nucleic acids selected from the group consisting of:

a nucleic acid comprising or consisting of a DNA sequence encoding a shmiR comprising an effector sequence set forth in SEQ ID NO: 15 and an effector complement sequence set forth in SEQ ID NO: 14 e.g., a nucleic acid comprising or consisting of a DNA sequence set forth in SEQ ID NO: 56 (shmiR2);

a nucleic acid comprising or consisting of a DNA sequence encoding a shmiR comprising an effector sequence set forth in SEQ ID NO: 17 and an effector complement sequence set forth in SEQ ID NO: 16 e.g., a nucleic acid comprising or consisting of a DNA sequence set forth in SEQ ID NO: 57 (shmiR3);

a nucleic acid comprising or consisting of a DNA sequence encoding a shmiR comprising an effector sequence set forth in SEQ ID NO: 21 and an effector complement sequence set forth in SEQ ID NO: 20 e.g., a nucleic acid comprising or consisting of a DNA sequence set forth in SEQ ID NO: 59 (shmiR5);

a nucleic acid comprising or consisting of a DNA sequence encoding a shmiR comprising an effector sequence set forth in SEQ ID NO: 27 and an effector complement sequence set forth in SEQ ID NO: 26 e.g., a nucleic acid comprising or consisting of a DNA sequence set forth in SEQ ID NO: 62 (shmiR9);

a nucleic acid comprising or consisting of a DNA sequence encoding a shmiR comprising an effector sequence set forth in SEQ ID NO: 31 and an effector complement sequence set forth in SEQ ID NO: 30 e.g., a nucleic acid comprising or consisting of a DNA sequence set forth in SEQ ID NO: 64 (shmiR13);

a nucleic acid comprising or consisting of a DNA sequence encoding a shmiR comprising an effector sequence set forth in SEQ ID NO: 33 and an effector complement sequence set forth in SEQ ID NO: 32 e.g., a nucleic acid comprising or consisting of a DNA sequence set forth in SEQ ID NO: 65 (shmiR14); and

a nucleic acid comprising or consisting of a DNA sequence encoding a shmiR comprising an effector sequence set forth in SEQ ID NO: 39 and an effector complement sequence set forth in SEQ ID NO: 38 e.g., a nucleic acid comprising or consisting of a DNA sequence set forth in SEQ ID NO: 68 (shmiR17).

In one example, each of the at least two nucleic acids in the ddRNAi construct encode a shmiR comprising an effector sequence which is substantially complementary to a region of corresponding length in an RNA transcript set forth in one of SEQ ID NOs: 2, 9, 10 and 13. Exemplary nucleic acids of the disclosure encoding shmiRs comprising effector sequences which are substantially complementary to regions of corresponding length in the RNA transcripts set forth in SEQ ID NO: 2, 9, 10 and 13 are described herein and shall be taken to apply mutatis mutandis to this example of the disclosure describing ddRNAi constructs.

In one example, the ddRNAi construct comprises at least two nucleic acids selected from the group consisting of:

a nucleic acid comprising or consisting of a DNA sequence encoding a shmiR comprising an effector sequence set forth in SEQ ID NO: 17 and an effector complement sequence set forth in SEQ ID NO: 16 e.g., a nucleic acid comprising or consisting of a DNA sequence set forth in SEQ ID NO: 57 (shmiR3);

a nucleic acid comprising or consisting of a DNA sequence encoding a shmiR comprising an effector sequence set forth in SEQ ID NO: 31 and an effector complement sequence set forth in SEQ ID NO: 30 e.g., a nucleic acid comprising or consisting of a DNA sequence set forth in SEQ ID NO: 64 (shmiR13);

a nucleic acid comprising or consisting of a DNA sequence encoding a shmiR comprising an effector sequence set forth in SEQ ID NO: 33 and an effector complement sequence set forth in SEQ ID NO: 32 e.g., a nucleic acid comprising or consisting of a DNA sequence set forth in SEQ ID NO: 65 (shmiR14); and

a nucleic acid comprising or consisting of a DNA sequence encoding a shmiR comprising an effector sequence set forth in SEQ ID NO: 39 and an effector complement sequence set forth in SEQ ID NO: 38 e.g., a nucleic acid comprising or consisting of a DNA sequence set forth in SEQ ID NO: 68 (shmiR17).

In one example, the ddRNAi construct of the disclosure comprises a nucleic acid encoding a shmiR comprising an effector sequence which is substantially complementary to a region of corresponding length in an RNA transcript set forth in SEQ ID NO: 9, and a nucleic acid encoding a shmiR comprising an effector sequence which is substantially complementary to a region of corresponding length in an RNA transcript set forth in SEQ ID NO: 13. For example, the ddRNAi construct may comprise:

(a) a nucleic acid comprising or consisting of a DNA sequence encoding a shmiR comprising an effector sequence set forth in SEQ ID NO: 31 and an effector complement sequence set forth in SEQ ID NO: 30 e.g., a nucleic acid comprising or consisting of a DNA sequence set forth in SEQ ID NO: 64 (shmiR13); and
(b) a nucleic acid comprising or consisting of a DNA sequence encoding a shmiR comprising an effector sequence set forth in SEQ ID NO: 39 and an effector complement sequence set forth in SEQ ID NO: 38 e.g., a nucleic acid comprising or consisting of a DNA sequence set forth in SEQ ID NO: 68 (shmiR17).

An exemplary ddRNAi construct of the disclosure comprises a nucleic acid comprising or consisting of a DNA sequence set forth in SEQ ID NO: 64 (shmiR13) and a nucleic acid comprising or consisting of a DNA sequence set forth in SEQ ID NO: 68 (shmiR17).

In one example, the ddRNAi construct comprises a nucleic acid encoding a shmiR comprising an effector sequence which is substantially complementary to a region of corresponding length in an RNA transcript set forth in SEQ ID NO: 2, and a nucleic acid encoding a shmiR comprising an effector sequence which is substantially complementary to a region of corresponding length in an RNA transcript set forth in SEQ ID NO: 10. For example, the ddRNAi construct may comprise:

(a) a nucleic acid comprising or consisting of a DNA sequence encoding a shmiR comprising an effector sequence set forth in SEQ ID NO: 17 and an effector complement sequence set forth in SEQ ID NO: 16, e.g., a nucleic acid comprising or consisting of a DNA sequence set forth in SEQ ID NO: 57 (shmiR3); and
(b) a nucleic acid comprising or consisting of a DNA sequence encoding a shmiR comprising an effector sequence set forth in SEQ ID NO: 33 and an effector complement sequence set forth in SEQ ID NO: 32 e.g., a nucleic acid comprising or consisting of the sequence set forth in SEQ ID NO:65 (shmiR14).

An exemplary ddRNAi construct of the disclosure comprises a nucleic acid comprising or consisting of a DNA sequence set forth in SEQ ID NO: 57 (shmiR3) and a nucleic acid comprising or consisting of a DNA sequence set forth in SEQ ID NO: 65 (shmiR14).

In each of the foregoing examples describing a ddRNAi construct of the disclosure, the or each nucleic acid comprised therein may be operably linked to a promoter. For example, the ddRNAi construct as described herein may comprise a single promoter which is operably-linked to the or each nucleic acid comprised therein e.g., to drive expression of one or more shmiRs and/or shRNAs from the ddRNAi construct.

In another example, each nucleic acid encoding a shmiR or shRNA of the disclosure comprised in the ddRNAi construct is operably-linked to a separate promoter.

According to an example in which multiple promoters are present, the promoters can be the same or different. For example, the construct may comprise multiple copies of the same promoter with each copy operably linked to a different nucleic acid of the disclosure. In another example, each promoter operably linked to a nucleic acid of the disclosure is different. For example, in a ddRNAi construct encoding two shmiRs, the two nucleic acids encoding the shmiRs are each operably linked to a different promoter. Equally, in an example in which a ddRNAi construct encodes one shmiR and one shRNA, the respective nucleic acids encoding the shmiR and shRNA are each operably linked to a different promoter.

In one example, the promoter is a constitutive promoter. The term “constitutive” when made in reference to a promoter means that the promoter is capable of directing transcription of an operably linked nucleic acid sequence in the absence of a specific stimulus (e.g., heat shock, chemicals, light, etc.). Typically, constitutive promoters are capable of directing expression of a coding sequence in substantially any cell and any tissue. The promoters used to transcribe shmiRs or shRNAs from the nucleic acid(s) of the disclosure include promoters for ubiquitin, CMV, β-actin, histone H4, EF-1α or pgk genes controlled by RNA polymerase II, or promoter elements controlled by RNA polymerase I.

In one example, a Pol II promoter such as CMV, SV40, U1, β-actin or a hybrid Pol II promoter is employed. Other suitable Pol II promoters are known in the art and may be used in accordance with this example of the disclosure. For example, a Pol II promoter system may be preferred in a ddRNAi construct of the disclosure which expresses a pri-miRNA which, by the action of the enzymes Drosha and Pasha, is processed into one or more shmiRs. A Pol II promoter system may also be preferred in a ddRNAi construct of the disclosure comprising sequence encoding a plurality of shRNAs or shmiRs under control of a single promoter. A Pol II promoter system may also be preferred where tissue specificity is desired.

In another example, a promoter controlled by RNA polymerase III is used, such as a U6 promoter (U6-1, U6-8, U6-9), H1 promoter, 7SL promoter, a human Y promoter (hY1, hY3, hY4 (see Maraia, et al., Nucleic Acids Res 22(15):3045-52(1994)) and hY5 (see Maraia, et al., Nucleic Acids Res 24(18):3552-59(1994)), a human MRP-7-2 promoter, an Adenovirus VA1 promoter, a human tRNA promoter, or a 5s ribosomal RNA promoter.

Suitable promoters for use in a ddRNAi construct of the disclosure are described in U.S. Pat. Nos. 8,008,468 and 8,129,510.

In one example, the promoter is a RNA pol III promoter. For example, the promoter is a U6 promoter (e.g., a U6-1, U6-8 or U6-9 promoter). In another example, the promoter is a H1 promoter.

In the case of a ddRNAi construct of the disclosure encoding a plurality of shmiRs, or encoding one or more shmiRs and a shRNA, as described herein, each of the nucleic acids in the ddRNAi construct is operably linked to a U6 promoter e.g., a separate U6 promoter.

In one example, the promoter in a construct is a U6 promoter. For example, the promoter is a U6-1 promoter. For example, the promoter is a U6-8 promoter. For example, the promoter is a U6-9 promoter.

In some examples, promoters of variable strength are employed. For example, use of two or more strong promoters (such as a Pol III-type promoter) may tax the cell, by, e.g., depleting the pool of available nucleotides or other cellular components needed for transcription. In addition, or alternatively, use of several strong promoters may cause a toxic level of expression of RNAi agents e.g., shmiRs or shRNAs, in the cell. Thus, in some examples one or more of the promoters in the multiple-promoter ddRNAi construct is weaker than other promoters in the construct, or all promoters in the construct may express the shmiRs or shRNAs at less than a maximum rate. Promoters may also be modified using various molecular techniques, or otherwise, e.g., through modification of various regulatory elements, to attain weaker levels or stronger levels of transcription. One means of achieving reduced transcription is to modify sequence elements within promoters known to control promoter activity. For example the Proximal Sequence Element (PSE) is known to effect the activity of human U6 promoters (see Domitrovich, et al., Nucleic Acids Res 31: 2344-2352 (2003). Replacing the PSE elements present in strong promoters, such as the human U6-1, U6-8 or U6-9 promoters, with the element from a weak promoter, such as the human U6-7 promoter, reduces the activity of the hybrid U6-1, U6-8 or U6-9 promoters. This approach has been used in the examples described in this application, but other means to achieve this outcome are known in the art.

Promoters useful in some examples of the present disclosure can be tissue-specific or cell-specific. The term “tissue specific” as it applies to a promoter refers to a promoter that is capable of directing selective transcription of a nucleic acid of interest to a specific type of tissue (e.g., tissue of the eye or muscle) in the relative absence of expression of the same nucleotide sequence of interest in a different type of tissue (e.g., liver). The term “cell-specific” as applied to a promoter refers to a promoter which is capable of directing selective transcription of a nucleic acid of interest in a specific type of cell in the relative absence of expression of the same nucleotide sequence of interest in a different type of cell within the same tissue. According to one example, a muscle-specific promoter is used, such as Spc512 or CK8. However, other muscle-specific promoters are known in the art and are contemplated for use in a ddRNAi construct of the disclosure.

In one example, a ddRNAi construct of the disclosure may additionally comprise one or more enhancers to increase expression of the shmiRs or shRNAs encoded by the nucleic acids described herein. Enhancers appropriate for use in examples of the present disclosure include the Apo E HCR enhancer, a CMV enhancer (Xia et al, Nucleic Acids Res 31-17(2003)), and other enhancers known to those skilled in the art. Suitable enhancers for use in a ddRNAi construct of the disclosure are described in U.S. Pat. No. 8,008,468.

In a further example, a ddRNAi construct of the disclosure may comprise a transcriptional terminator linked to a nucleic acid encoding a shmiR or shRNA of the disclosure. In the case of a ddRNAi construct comprising a plurality of nucleic acids described herein i.e., encoding multiple shmiRs and/or shRNAs, the terminators linked to each nucleic acid can be the same or different. For example, in a ddRNAi construct of the disclosure in which a RNA pol III promoter is employed, the terminator may be a contiguous stretch of 4 or more or 5 or more or 6 or more T residues. However, where different promoters are used, the terminators can be different and are matched to the promoter from the gene from which the terminator is derived. Such terminators include, but are not limited to, the SV40 poly A, the AdV VA1 gene, the 5S ribosomal RNA gene, and the terminators for human t-RNAs. Other promoter and terminator combinations are known in the art and are contemplated for use in a ddRNAi construct of the disclosure.

In addition, promoters and terminators may be mixed and matched, as is commonly done with RNA pol II promoters and terminators.

In one example, the promoter and terminator combinations used for each nucleic acid in a ddRNAi construct comprising a plurality of nucleic acids is different to decrease the likelihood of DNA recombination events between components.

One exemplary ddRNAi construct of the disclosure comprises a nucleic acid comprising or consisting of a DNA sequence encoding shmiR13 as described herein operably linked to a promoter, and a nucleic acid comprising or consisting of a DNA sequence encoding shmiR17 as described herein operably linked to a promoter. For example, an exemplary ddRNAi construct of the disclosure comprises a nucleic acid comprising or consisting of a DNA sequence set forth in SEQ ID NO: 64 operably linked to a promoter, and a nucleic acid comprising or consisting of a DNA sequence set forth in SEQ ID NO: 68 operably linked to a promoter. In one example, each nucleic acid in the ddRNAi construct encoding a shmiR is operably linked to a separate promoter. In another example, each nucleic acid in the ddRNAi construct encoding a shmiR is operably linked to the same promoter. For example, the or each promoter may be a U6 promoter e.g., a U6-1, U6-8 or U6-9 promoter. For example, the or each promoter may be a muscle specific promoter e.g., a Spc512 or CK8 promoter.

In accordance with an example in which the nucleic acids in the ddRNAi construct encoding shmiR13 and shmiR17 are operably-linked to the same Spc512 promoter, the ddRNAi construct comprises or consists of the DNA sequence set forth in SEQ ID NO: 72. In accordance with an example in which the nucleic acids in the ddRNAi construct encoding shmiR13 and shmiR17 are operably-linked to the same CK8 promoter, the ddRNAi construct comprises or consists of the DNA sequence set forth in SEQ ID NO: 70.

Another exemplary ddRNAi construct of the disclosure comprises a nucleic acid comprising or consisting of a DNA sequence encoding shmiR3 as described herein operably linked to a promoter, and a nucleic acid comprising or consisting of a DNA sequence encoding shmiR14 as described herein operably linked to a promoter. For example, an exemplary ddRNAi construct of the disclosure comprises a nucleic acid comprising or consisting of a DNA sequence set forth in SEQ ID NO: 57 operably linked to a promoter, and a nucleic acid comprising or consisting of a DNA sequence set forth in SEQ ID NO: 65 operably linked to a promoter. In one example, each nucleic acid in the ddRNAi construct encoding a shmiR is operably linked to a separate promoter. In another example, each nucleic acid in the ddRNAi construct encoding a shmiR is operably linked to the same promoter. For example, the or each promoter may be a U6 promoter e.g., a U6-1, U6-8 or U6-9 promoter. For example, the or each promoter may be a muscle specific promoter e.g., a Spc512 or CK8 promoter.

In accordance with an example in which the nucleic acids in the ddRNAi construct encoding shmiR3 and shmiR14 are operably-linked to the same Spc512 promoter, the ddRNAi construct comprises or consists of the DNA sequence set forth in SEQ ID NO: 71. In accordance with an example in which the nucleic acids in the ddRNAi construct encoding shmiR3 and shmiR14 are operably-linked to the same CK8 promoter, the ddRNAi construct comprises or consists of the DNA sequence set forth in SEQ ID NO: 69.

Also provided is a plurality of ddRNAi constructs. For example, a plurality of nucleic acids as encoding shmiRs as described herein may be provided within a plurality of ddRNAi constructs, wherein each ddRNAi construct comprises one or more of the plurality of nucleic acids described herein. Combinations of nucleic acids encoding shmiR have been described and shall be taken to apply mutatis mutandis to this example of the disclosure. In one example, each nucleic acid in the plurality of nucleic acids described herein is provided within its own ddRNAi construct.

According to any example in which a plurality of ddRNAi constructs is provided, each ddRNAi construct may also comprise one or more promoters operably linked to the nucleic acid(s) encoding the shmiR(s) comprised therein. In one example, each ddRNAi construct comprises a single nucleic acid encoding a shmiR and a promoter operably linked thereto. According to an example in which one or more of the plurality of ddRNAi constructs comprises two or more nucleic acid encoding shmiRs, each nucleic acid in the one or more ddRNAi constructs is operably linked to a separate promoter. In another example in which one or more of the plurality of ddRNAi constructs comprises two or more nucleic acid encoding shmiRs, the two or more nucleic acids are operably linked to the same promoter in the ddRNAi construct.

One exemplary plurality of ddRNAi constructs of the disclosure comprises a ddRNAi construct comprising a nucleic acid comprising or consisting of a DNA sequence encoding shmiR13 as described herein operably linked to a promoter, and a ddRNAi construct comprising a nucleic acid comprising or consisting of a DNA sequence encoding shmiR17 as described herein operably linked to a promoter. For example, an exemplary plurality of ddRNAi constructs of the disclosure comprises a ddRNAi construct comprising a nucleic acid comprising or consisting of a DNA sequence set forth in SEQ ID NO: 64 operably linked to a promoter, and a ddRNAi construct comprising a nucleic acid comprising or consisting of a DNA sequence set forth in SEQ ID NO: 68 operably linked to a promoter. In one example, the promoters are U6 promoters e.g., selected from a U6-1, U6-8 or U6-9 promoter. In another example, the promoters are muscle specific promoters e.g., Spc512 or CK8 promoters.

Another exemplary plurality of ddRNAi constructs of the disclosure comprises a ddRNAi construct comprising a nucleic acid comprising or consisting of a DNA sequence encoding shmiR3 as described herein operably linked to a promoter, and a ddRNAi construct comprising a nucleic acid comprising or consisting of a DNA sequence encoding shmiR14 as described herein operably linked to a promoter. For example, an exemplary plurality of ddRNAi constructs of the disclosure comprises a ddRNAi construct comprising a nucleic acid comprising or consisting of a DNA sequence set forth in SEQ ID NO: 57 operably linked to a promoter, and a ddRNAi construct comprising a nucleic acid comprising or consisting of a DNA sequence set forth in SEQ ID NO: 65 operably linked to a promoter. In one example, the promoters are U6 promoters e.g., selected from a U6-1, U6-8 or U6-9 promoter. In another example, the promoters are muscle specific promoters e.g., Spc512 or CK8 promoters.

In addition, the or each ddRNAi construct can comprise one or more multiple cloning sites and/or unique restriction sites that are located strategically, such that the promoter, nucleic acid encoding the shmiR or shRNA and/or other regulator elements are easily removed or replaced. The or each ddRNAi construct can be assembled from smaller oligonucleotide components using strategically located restriction sites and/or complementary sticky ends. The base vector for one approach according to the present disclosure comprises plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into e.g., an AdV backbone or an AAV backbone using two unique restriction enzyme sites (the same or different ones) that flank the single-, double- or multiple-expression cassette insert.

Generation of the or each ddRNAi construct can be accomplished using any suitable genetic engineering techniques known in the art, including without limitation, the standard techniques of PCR, oligonucleotide synthesis, restriction endonuclease digestion, ligation, transformation, plasmid purification, and DNA sequencing. If the or each construct is a viral construct, the construct comprises, for example, sequences necessary to package the ddRNAi construct into viral particles and/or sequences that allow integration of the ddRNAi construct into the target cell genome. In some examples, the or each viral construct additionally contains genes that allow for replication and propagation of virus, however such genes will be supplied in trans. Additionally, the or each viral construct cam contain genes or genetic sequences from the genome of any known organism incorporated in native form or modified. For example, a viral construct may comprise sequences useful for replication of the construct in bacteria.

The or each construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines.

Other genetic elements that may find use in embodiments of the present disclosure include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the or each construct, an internal ribosomal entry site (IRES) sequence can be included. In one example, the additional genetic elements are operably linked with and controlled by an independent promoter/enhancer. In addition a suitable origin of replication for propagation of the construct in bacteria may be employed. The sequence of the origin of replication generally is separated from the ddRNAi construct and other genetic sequences. Such origins of replication are known in the art and include the pUC, ColE1, 2-micron or SV40 origins of replication.

### Expression Vectors

In one example, a ddRNAi construct of the disclosure is included within an expression vector.

In one example, the expression vector is a plasmid e.g., as is known in the art. In one example, a suitable plasmid expression vector is a pAAV vector e.g., a self-complementary pAAV (pscAAV) plasmid vector or single-stranded pAAV (pssAAV) plasmid vector. As described herein, the plasmid may comprise one or more promoters (suitable examples of which are described) to drive expression of one or more shmiRs of the disclosure.

In one example, the expression vector is mini-circle DNA. Mini-circle DNA is described in U.S. Patent Publication No. 2004/0214329. Mini-circle DNA are useful for persistently high levels of nucleic acid transcription. The circular vectors are characterized by being devoid of expression-silencing bacterial sequences. For example, mini-circle vectors differ from bacterial plasmid vectors in that they lack an origin of replication, and lack drug selection markers commonly found in bacterial plasmids, e.g. β-lactamase, tet, and the like. Consequently, minicircle DNA becomes smaller in size, allowing more efficient delivery.

In one example, the expression vector is a viral vector.

A viral vector based on any appropriate virus may be used to deliver a ddRNAi of the disclosure. In addition, hybrid viral systems may be of use. The choice of viral delivery system will depend on various parameters, such as the tissue targeted for delivery, transduction efficiency of the system, pathogenicity, immunological and toxicity concerns, and the like.

Commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses). In one example, a viral vector of the disclosure integrates into a host cell's chromatin. In another example, a viral vector of the disclosure persists in a host cell's nucleus as an extrachomosomal episome.

In one example, a viral vector is an adenoviral (AdV) vector. Adenoviruses are medium-sized double-stranded, non-enveloped DNA viruses with linear genomes that is between 26-48 Kbp. Adenoviruses gain entry to a target cell by receptor-mediated binding and internalization, penetrating the nucleus in both non-dividing and dividing cells. Adenoviruses are heavily reliant on the host cell for survival and replication and are able to replicate in the nucleus of vertebrate cells using the host's replication machinery.

In one example, a viral vector is from the Parvoviridae family. The Parvoviridae is a family of small single-stranded, non-enveloped DNA viruses with genomes approximately 5000 nucleotides long. Included among the family members is adeno-associated virus (AAV). In one example, a viral vector of the disclosure is an AAV. AAV is a dependent parvovirus that generally requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells. Because progeny virus is not produced from AAV infection in the absence of helper virus, the extent of transduction is restricted only to the initial cells that are infected with the virus. It is this feature which makes AAV a desirable vector for the present disclosure. Furthermore, unlike retrovirus, adenovirus, and herpes simplex virus, AAV appears to lack human pathogenicity and toxicity (Kay, et al., Nature. 424: 251 (2003)). Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as shmiRs and shRNAs. Preferably the AAV used as a expression vector and delivery system is from a serotype which is capable of infecting humans e.g., an AAV selected from the group consisting of AAV serotype 1, 2, 3, 4, 5, 6, 7, 8, 9, 10, 11, 12 and 13. In one particular example, an AAV of serotype 8 or 9 is used as a vector. In one example, the AAV is from serotype 8. In another example, the AAV is from serotype 9. In accordance with any example in which the AAV is from a serotype other than serotype 2, the AAV may comprise AAV serotype 2 inverted terminal repeats (ITRs) e.g., to improve transduction efficiency of the AAV. Alternatively, or in addition, the AAV may comprise a modified capsid protein e.g., to assist with production of the AAV in insect cells using a baculovirus system. For example, the AAV may comprise a viral capsid protein comprising a subunit 1 (VP1) with a modified phospholipase (PL) domain sequence. For example, the PL domain of the VP1 may comprise a sequence comprising a serine at position 1, a glutamic acid at position 26, an arginine at position 40, an aspartic acid at position 43, a serine at position 44 and a lysine at position 64, wherein the amino acid positions are defined relative to the unmodified sequence set forth in SEQ ID NO: 88, wherein the amino acids at any one or more of positions 1, 26, 40, 43, 44 and 64 are modified relative to a corresponding wildtype sequence.

In one example, the viral vector is an AAV from serotype 8, or an AAV pseudotyped with a serotype 8 capsid, comprising ITRs from AAV serotype 2 and a modified capsid protein in which the VP1 comprises a PL domain sequence comprises a serine at position 1, a glutamic acid at position 26, an arginine at position 40, an aspartic acid at position 43, a serine at position 44 and a lysine at position 64, wherein the amino acid positions are defined relative to the sequence set forth in SEQ ID NO: 88. For example, the modified capsid protein from AAV8 may comprise a VP1 comprising a PL domain comprising the sequence set forth in SEQ ID NO: 89. In another example, the viral vector is an AAV from serotype 9, or an AAV pseudotyped with a serotype 9 capsid, comprising ITRs from AAV serotype 2 and a modified capsid protein in which the VP1 comprises a PL domain sequence comprises a serine at position 1, a glutamic acid at position 26, an arginine at position 40, an aspartic acid at position 43 and a serine at position 44 and a lysine at position 64, wherein the amino acid positions are defined relative to the unmodified sequence set forth in SEQ ID NO: 88. For example, the modified capsid protein from AAV9 may comprise a VP1 comprising a PL domain comprising the sequence set forth in SEQ ID NO: 90.

Methods of producing AAV suitable for use in gene therapy (e.g., replication incompetent AAV) are well known in the art and contemplated herein. For example, AAV may be produced in insect cells using a baculovirus system, for example, as described in US20120028357 A1, WO2007046703, US20030148506 A1, WO2017184879, US20040197895 A1 and WO2007148971, the content of which is described by reference herein. Recombinant AAV may also be produced in mammalian cells, both adherent and suspension cells, methods for which are described in WO2015031686, WO2009097129, WO2007127264, WO1997009441 and WO2001049829, the content of which is described by reference herein. Methods of producing recombinant AAV for use in gene therapy are also described in Berns KI and Giraud C (1996) Biology of adeno-associated virus. Curr Top Microbiol Immunol 218:1-23, Snyder and Flotte (2002) Curr. Opin. Biotechnol., 13:418-423, and Synder R O and Moullier P, Adeno-associated virus; methods and protocols. New York: Humana Press (2011), the contents of which are incorporated by reference herein.

Another viral delivery system useful with the ddRNAi constructs of the disclosure is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some examples, a viral vector is a lentivirus. Lentivirus vectors are often pseudotyped with vesicular somatitis virus glycoprotein (VSV-G), and have been derived from the human immunodeficiency virus (HIV); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, this modification eliminates transcription from integrated proviruses required for vector mobilization. One of the main advantages to the use of lentiviral vectors is that gene transfer is persistent in most tissues or cell types, even following cell division of the transduced cell.

A lentiviral-based construct used to express shmiRs and/or shRNAs from the nucleic acids and ddRNAi constructs of the disclosure comprises sequences from the 5′ and 3′ long terminal repeats (LTRs) of a lentivirus. In one example, the viral construct comprises an inactivated or self-inactivating 3′ LTR from a lentivirus. The 3′ LTR may be made self-inactivating by any method known in the art. For example, the U3 element of the 3′ LTR contains a deletion of its enhancer sequence, e.g., the TATA box, Sp1 and NF-kappa B sites. As a result of the self-inactivating 3′ LTR, the provirus that is integrated into the host genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the ddRNAi or nucleic acid of the present invention to cells of interest, including but not limited to gene-deleted adenovirus-transposon vectors (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus.

Testing a shmiR or ddRNAi Construct of the Disclosure

### Cell Culture Models

An example of cell line useful as a cell culture model for OPMD is the HEK293T cell line (HEK293T, ATCC, Manassas, USA) which has been transfected with a vector expressing normal Ala10-humanPABPN1-FLAG (Ala10) or mutant Ala17-humanPABPN1-FLAG (Ala17), the latter being hallmark of OPMD.

Further examples of cell lines useful as cell culture models for OPMD are the C2C12 mouse muscle cell and the ARPE-19 human retinal cells.

Another example of a cell line useful as a cell culture model for OPMD is the primary mouse myoblast (IM2) cell line stably transfected to express either normal Ala10-humanPABPN1-FLAG (Ala10) or mutant Ala17-humanPABPN1-FLAG (Ala17). An exemplary IM2 derived cell line which stably expresses mutant Ala17-humanPABPN1-FLAG (Ala17) is the H2 kB-D7e cell line. The H2kB-D7e cell line is also described in Raz et al., (2011) American Journal of Pathology, 179(4):1988-2000.

Other cell lines suitable for cell culture models of OPMD are known in the art, such as described in Fan et al., (2001) Human Molecular Genetics, 10:2341-2351, Bao et al., (2002) The Journal of Biological Chemistry, 277:12263-12269, and Abu-Baker et al., (2003) Human Molecular Genetics, 12:2609-2623.

As exemplified herein, activity of a shmiR of the disclosure is determined by administering a nucleic acid encoding the shmiR, or a ddRNAi construct or expression vector comprising same, to the cell and subsequently measuring the level of expression of a RNA or protein encoded by the PABPN1 gene. For example, intracellular PABPN1 gene expression can be assayed by any one or more of RT-PCR, quantitative PCR, semi-quantitative PCR, or in-situ hybridization under stringent conditions, using one or more probes or primers which are specific for PABPN1. PABPN1 mRNA or DNA can also be assayed either by PCR using one or more probes or primers which are specific for PABPN1 or Western blots or ELISA can be used to detect PABPN1 protein.

Polynucleotides which may be used in RT-PCR, quantitative PCR or semi-quantitative PCR techniques for detecting PABPN1 expression are known and commercially available (Thermo Fisher). However, polynucleotides useful for PCR-based detection methods can be designed based on sequence information available for PABPN1 using method and/or software known in the art. In one example, the presence or absence of PABPN1 mRNA may be detected using RT-PCR using standard methodologies known in the art. In one example, the presence or absence or relative amount of PABPN1 polypeptide or protein may be detected using any one or more of Western blotting, ELISA, or other standard quantitative or semiquantitative techniques available in the art, or a combination of such techniques. Techniques relying on antibody recognition of PABPN1 are contemplated and are described herein. In one example, the presence or absence or relative abundance of PABPN1 polypeptide may be detected with techniques which comprise antibody capture of PABPN1 polypeptides in combination with electrophoretic resolution of captured PABPN1 polypeptides, for example using the Isonostic™ Assay (Target Discovery, Inc.). Antibodies are commercially available for PABPN1 protein.

Various means for normalizing differences in transfection or transduction efficiency and sample recovery are known in the art.

A nucleic acid, ddRNAi construct or expression vector of the disclosure that reduces expression of a mRNA or protein encoded by PABPN1 or that reduces the presence of nuclear aggregates of PABPN1 protein, relative to a level of mRNA expression or protein encoded by PABPN1 or an amount of nuclear aggregates of PABPN1 protein in the absence of the RNA of the disclosure, is considered to be useful for therapeutic applications e.g., such as treating OPMD by reducing expression of endogenous PABPN1 and replacing some or all of the endogenous PABPN1 with a PABPN1 protein which is not causative of OPMD as described herein.

### Animal Models

There are several small animal models available for studying OPMD, examples of which are described in Uyama et al., (2005) Acta Myologica, 24(2):84-88 and Chartier and Simonelig (2013) Drug Discovery Today: technologies, 10:e103-107. An exemplary animal model is the A17.1 transgenic mouse model which has been described previously in Davies et al., (2005) Nature Medicine, 11:672-677 and Trollet et al., (2010) Human Molecular Genetics, 19(11):2191-2207.

Any of the foregoing animal models can be used to determine the efficacy of a shmiR or ddRNAi construct of the disclosure to knockdown, reduce or inhibit expression of a RNA or protein encoded by the PABPN1 gene.

Methods for assaying PABPN1 gene expression have been described herein with respect to cell models and shall be taken to apply mutatis mutandis to this example of the disclosure.

### Agents for Replacement of Functional PABPN1

In one example, the present disclosure provides an agent for replacement of functional PABPN1 protein e.g., to a cell or animal. The functional PABPN1 protein will not be causative of OPMD, nor will it be encoded by a mRNA transcript which is targeted by the shmiR(s) or shRNA(s) of the disclosure.

In one example, the agent for replacement of functional PABPN1 protein to a cell or animal is a nucleic acid e.g., such as DNA or cDNA, encoding the functional PABPN1 protein. For example, the nucleic acid encoding the functional PABPN1 protein may be codon optimised e.g., contain one or more degenerate or wobble bases relative to the wild type PABPN1 nucleic acid but which encodes for identical amino acids, so that the corresponding mRNA sequence coding for the functional PABPN1 protein is not recognised by the shmiR(s) or shRNA(s) of the disclosure. For example, a codon optimised nucleic acid encoding the functional PABPN1 protein may comprise one or more degenerate or wobble bases relative to the wild type PABPN1 nucleic acid within the region targeted by the shmiR(s) or shRNA(s) of the disclosure. In one example, the one or more degenerate or wobble bases resides within a seed region of an effector sequence a shmiR or shRNA of the disclosure.

In one example, a nucleic acid encoding the functional PABPN1 protein is codon optimised such that its corresponding mRNA sequence is not recognised by the shmiR(s) or shRNA(s) of the disclosure. Preferably, the functional PABPN1 protein encoded by the codon optimised nucleic acid sequence comprises the amino acid sequence set forth in SEQ ID NO: 74 i.e., the amino acid sequence of the wild-type human PABPN1 protein. A skilled person will appreciate that there are a number of nucleotide sequence combinations which may be used to encode functional PABPN1 protein, and the choice of nucleotide sequence will ultimately depend on the effector sequence of the shmiR(s) or shRNA(s) i.e., such that the codon-optimised nucleic acid is not recognised by the shmiR(s) or shRNA(s). In one example, the agent for replacement of functional PABPN1 protein is a nucleic acid comprising the sequence set forth in SEQ ID NO: 73. In one example, the nucleic acid encoding the functional PABPN1 protein may also comprise a Kozak sequence.

In one example, the codon-optimised nucleic acid encoding the functional PABPN1 protein is operably-linked to a promoter suitable for expression of the functional PABPN1 protein. Promoters suitable for expression of the functional PABPN1 protein in muscle may be particularly suitable. One exemplary promoter suitable for use with the nucleic acid encoding the functional PABPN1 protein is a Spc512 promoter. Another exemplary promoter suitable for use with the nucleic acid encoding the functional PABPN1 protein is a CK8 promoter. However, any suitable promoter known in the art may be used. For example, other suitable promoters for use with the nucleic acid encoding the functional PABPN1 protein are described in US 20110212529 A1.

As described herein, promoters useful in some examples of the present disclosure can be tissue-specific or cell-specific.

In one example, a codon-optimised nucleic acid encoding the functional PABPN1 protein of the disclosure may additionally comprise one or more enhancers to increase expression of the functional PABPN1 protein and its corresponding mRNA transcript. Enhancers appropriate for use in this example of the present disclosure will be known to those skilled in the art.

A nucleic acid encoding the functional PABPN1 protein may be comprised within an expression vector. Exemplary expression vectors have been described in the context of nucleic acid and ddRNAi constructs of the disclosure and shall be taken to apply mutatis mutandis to this example.

Accordingly, in one example, an agent for replacement of functional PABPN1 protein to a cell or animal may be an expression vector comprising a codon-optimised nucleic acid encoding the functional PABPN1 protein. For example, an expression vector of the disclosure may comprise the codon-optimised nucleic acid encoding the functional PABPN1 protein and a promoter for expression therefor e.g., a SpC512 promoter or a CK8 promter. In one example, the codon optimised nucleic acid encoding the functional PABPN1 protein may also comprise a Kozak sequence.

In one example, the nucleic acid encoding the functional PABPN1 protein as described herein may be comprised within a plasmid expression vector. Suitable plasmid expression vectors have been described herein and will be known in the art. In one example, a suitable plasmid expression vector is a pAAV vector e.g., a pscAAV plasmid vector or pssAAV plasmid vector.

In one example, the expression vector is mini-circle DNA. Mini-circle DNA vectors have been described herein.

In one example, the expression vector is a viral vector. For example, a viral vector based on any appropriate virus may be used to deliver a codon optimised nucleic acid encoding the functional PABPN1 protein of the disclosure. In addition, hybrid viral systems may be of use. The choice of viral delivery system will depend on various parameters, such as the tissue targeted for delivery, transduction efficiency of the system, pathogenicity, immunological and toxicity concerns, and the like.

Exemplary viral systems for delivery of genetic material to a cell or animal have been described in the context of the RNAs and ddRNAi constructs of the disclosure and shall be taken to apply mutatis mutandis to this example.

In one example, the viral vector is an AAV (e.g., AAV9 or a modified AAV9).

In one example, the viral vector is an AdV vector.

In one example, the viral vector is a lentivirus.

Other viral or non-viral systems known to those skilled in the art may be used to deliver the codon-optimised nucleic acid encoding functional PABPN1 protein of the present disclosure to cells of interest, including but not limited to gene-deleted adenovirus-transposon vectors (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus.

In accordance with an example in which the codon-optimised nucleic acid encoding the functional PABPN1 protein as described herein is provided with a nucleic acid, ddRNAi construct or expression vector of the disclosure, the codon-optimised nucleic acid encoding the functional PABPN1 protein may be comprised within the same expression vector as the nucleic acid or ddRNAi construct. Thus, the codon-optimised nucleic acid encoding the functional PABPN1 protein and the nucleic acid or ddRNAi construct of the disclosure may be provided as a single DNA construct e.g., within an expression vector.

In an alternative example in which a codon-optimised nucleic acid encoding functional PABPN1 protein of the disclosure and a nucleic acid or ddRNAi construct of the disclosure are to be provided together, the codon-optimised nucleic acid encoding functional PABPN1 protein and the nucleic acid or ddRNAi construct may be comprised within different expression vectors. Where the codon-optimised nucleic acid encoding functional PABPN1 protein and the nucleic acid or ddRNAi construct are comprised within different expression vectors, the respective expression vectors may be the same type of vector or be different types of vectors.

### Testing for Functional PABPN1

### Animal Models

Exemplary animal models for studying OPMD have been described.

Any of the foregoing animal models can be used to determine the efficacy of an agent of the disclosure to replace functional PABPN1 protein in vivo in the presence of one or more nucleic acid(s), ddRNAi construct(s) or expression vector(s) of the disclosure (expressing one or more shmiR(s) of the disclosure).

Methods for assaying PABPN1 expression have been described herein with respect to cell models and shall be taken to apply mutatis mutandis to this example of the disclosure.

In one example, histological and morphological analyses may be used to determine the efficacy of an agent of the disclosure to replace functional PABPN1 protein in vivo in the presence one or more nucleic acid(s), ddRNAi construct(s) or expression vector(s) of the disclosure (expressing one or more shmiR(s) of the disclosure). Further assays which may be used to determine efficacy of an agent of the disclosure to replace functional PABPN1 protein in vivo are described in Trollet et al., (2010) Human Molecular Genetics, 19(11): 2191-2207.

Single DNA Constructs for ddRNAi and Replacement of Functional PABPN1

The present disclosure also provides a single DNA construct comprising the nucleic acid encoding the functional PABPN1 protein as described herein and one or more ddRNAi construct(s) of the disclosure. An exemplary DNA construct comprising a nucleic acid encoding the functional PABPN1 protein and the ddRNAi construct of the disclosure is described in Example 2. In one example, the DNA construct may comprise a single ddRNAi construct as described herein in combination with the nucleic acid encoding the functional PABPN1 protein. In another example, the DNA construct may comprise a plurality of ddRNAi constructs in combination with the nucleic acid encoding the functional PABPN1 protein. In each example of the DNA construct, the DNA sequence encoding the functional PABPN1 protein is codon optimised such that its mRNA transcript is not targeted by the shmiR(s) of the ddRNAi construct(s).

In one example, functional PABPN1 protein is a wild-type human PABPN1 protein e.g., having a sequence set forth in SEQ ID NO: 74. It will be appreciated that the codon optimised DNA sequence encoding the functional PABPN1 protein may vary depending on the shmiR(s) encoded by the ddRNAi construct. That is, the specific codons within the PABPN1 mRNA transcript to be modified may vary depending on the effector sequence(s) of shmiR(s) encoded by the ddRNAi construct. In one example a codon optimised DNA sequence encoding the functional PABPN1 protein is set forth in SEQ ID NO: 73.

The DNA construct may also comprise one or more promoters e.g., to drive expression of the functional PABPN1 protein and/or shmiRs encoded by the ddRNAi construct. Promoters useful in some examples of the present disclosure can be tissue-specific or cell-specific. Exemplary promoters for use in the DNA constructs of the disclosure are muscle-specific promoter, such as for example, Spc512 and CK8. However, any suitable promoter known in the art is contemplated for use in the DNA construct described herein e.g., such as those described in US 20110212529 A1.

The DNA construct may be provided in the form of an expression vector or may be comprised within an expression vector. Suitable expression vectors have been described herein and will be known in the art.

In one example, the expression vector is a viral vector. For example, a viral vector based on any appropriate virus may be used to deliver the single DNA construct of the disclosure. In addition, hybrid viral systems may be of use. The choice of viral delivery system will depend on various parameters, such as the tissue targeted for delivery, transduction efficiency of the system, pathogenicity, immunological and toxicity concerns, and the like.

In another example, a suitable plasmid expression vector is a pAAV vector e.g., a pscAAV plasmid vector or pssAAV plasmid vector. Other exemplary viral systems for delivery of genetic material to a cell or animal have been described in the context of the ddRNAi constructs of the disclosure and shall be taken to apply mutatis mutandis to this example.

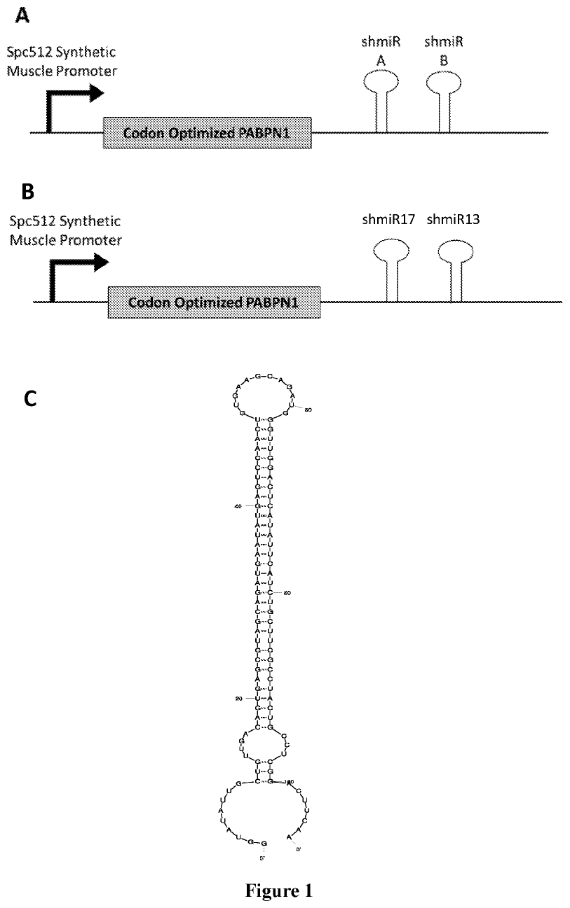

In one example, the DNA construct is provided in the form of a pAAV expression vector comprising, in a 5′ to 3′ direction, a muscle-specific promoter e.g., a Spc512 promoter, a ddRNAi construct as described herein and a PABPN1 construct described herein, e.g., wherein the ddRNAi construct is positioned in the 3′ untranslated region (UTR) of nucleic acid encoding the functional PABPN1 protein. A DNA construct in accordance with this example is illustrated in FIG. 1A.

An exemplary DNA construct in accordance with this example is a pAAV expression vector comprising, in a 5′ to 3′ direction:

(a) a muscle-specific promoter e.g., Spc512;
(b) a PABPN1 construct as described herein comprising a DNA sequence encoding a functional PABPN1 protein having a mRNA transcript which is not targeted by the shmiRs encoded by the ddRNAi construct; and
(c) a ddRNAi construct of the disclosure comprising a nucleic acid comprising a DNA sequence encoding shmiR17 as described herein and a nucleic acid comprising a DNA sequence encoding shmiR13 as described herein.

In accordance with this example, the DNA construct may comprise or consist of the DNA sequence set forth in SEQ ID NO: 72.

An exemplary ddRNAi construct encoding shmiR13 and shmiR17 for inclusion in a DNA construct of the disclosure comprises a nucleic acid comprising or consisting of a DNA sequence encoding a shmiR comprising an effector sequence set forth in SEQ ID NO: 31 and an effector complement sequence which is substantially complementary to the sequence set forth in SEQ ID NO: 31 e.g., an effector complement sequence set forth in SEQ ID NO: 30 (shmiR13), and a nucleic acid comprising or consisting of a DNA sequence encoding a shmiR comprising an effector sequence set forth in SEQ ID NO: 39 and an effector complement sequence which is substantially complementary to the sequence set forth in SEQ ID NO: 39 e.g., an effector complement sequence set forth in SEQ ID NO: 38 (shmiR17). For example, the ddRNAi construct in accordance with this example of the DNA construct may comprise a nucleic acid comprising or consisting of the DNA sequence set forth in SEQ ID NO: 64 (shmiR13), and a nucleic acid comprising or consisting of the DNA sequence set forth in SEQ ID NO: 68 (shmiR17).

Whilst certain examples have been described, it will be appreciated that a DNA construct in accordance with the present disclosure may include any ddRNAi construct described herein encoding one or more shmiRs. For example, ddRNAi constructs encoding shmiRs described in Examples 1 to 5 may be particularly suitable for inclusion in a DNA construct of the disclosure.

### Compositions and Carriers

In some examples, the nucleic acid(s), ddRNAi construct(s), DNA construct, or expression vector(s) of the disclosure is/are provided in a composition. In some examples, a nucleic acid encoding a functional PABPN1 protein of the disclosure is provided in a composition. In some example, the nucleic acid(s), ddRNAi construct(s) or expression vector(s) of the disclosure is/are provided in a composition together with a nucleic acid encoding a functional PABPN1 protein of the disclosure. In some examples, the one or more nucleic acid(s) or ddRNAi construct(s) and the nucleic acid encoding a functional PABPN1 protein are provided in the same expression vector within a composition (e.g., within a DNA construct of the disclosure).

As described herein, the expression vector may comprise a ddRNAi construct of the disclosure alone or in combination with a codon-optimised nucleic acid encoding the functional PABPN1 protein of the disclosure. Reference herein to an expression vector and/or a composition comprising same will therefore be understood to encompass: (i) an expression vector comprising a ddRNAi construct of the disclosure, or a composition comprising same; (ii) an expression vector comprising both of a ddRNAi construct of the disclosure and a codon-optimised nucleic acid encoding the functional PABPN1 protein of the disclosure, or a composition comprising same; or (iii) an expression vector comprising a codon-optimised nucleic acid encoding the functional PABPN1 protein of the disclosure, or a composition comprising same.

Accordingly, a composition of the disclosure may comprise (i) an expression vector comprising a ddRNAi construct of the disclosure, and (ii) an expression vector comprising a codon-optimised nucleic acid encoding the functional PABPN1 protein of the disclosure. Alternatively, a composition of the disclosure may comprise a single expression vector comprising ddRNAi construct of the disclosure and a codon-optimised nucleic acid encoding the functional PABPN1 protein of the disclosure.

In yet another example, an expression vector comprising a ddRNAi construct of the disclosure may be provided in one composition and an expression vector comprising a codon-optimised nucleic acid encoding the functional PABPN1 protein of the disclosure may be provided within another composition e.g., which are packaged together.

A composition of the disclosure may also comprise one or more pharmaceutically acceptable carriers or diluents. For example, the composition may comprise a carrier suitable for delivery of the nucleic acid(s), ddRNAi construct(s), DNA construct, or expression vector(s) of the disclosure to muscle of a subject following administration thereto.

In some examples, the carrier is a lipid-based carrier, cationic lipid, or liposome nucleic acid complex, a liposome, a micelle, a virosome, a lipid nanoparticle or a mixture thereof.

In some examples, the carrier is a biodegradable polymer-based carrier, such that a cationic polymer-nucleic acid complex is formed. For example, the carrier may be a cationic polymer microparticle suitable for delivery of one or more nucleic acid(s), ddRNAi construct(s), DNA construct, or expression vector(s) of the disclosure to muscle cells or tissue of the eye. Use of cationic polymers for delivery compositions to cells is known in the art, such as described in Judge et al. Nature 25: 457-462 (2005), the contents of which is incorporated herein by reference. An exemplary cationic polymer-based carrier is a cationic DNA binding polymer, such as polyethylenimine. Other cationic polymers suitable for complexing with, and delivery of nucleic acid(s), ddRNAi construct(s), or expression vector(s) of the disclosure include poly(L-lysine) (PLL), chitosan, PAMAM dendrimers, and poly(2-dimethylamino)ethyl methacrylate (pDMAEMA). Other polymers include poly beta-amino esters. These are other suitable cationic polymers are known in the art and are described in Mastrobattista and Hennink, Nature Materials, 11:10-12 (2012), WO/2003/097107 and WO/2006/041617, the full contents of which are incorporated herein by reference. Such carrier formulations have been developed for various delivery routes including parenteral subcutaneous injection, intravenous injection and inhalation.

In a further example, the carrier is a cyclodextrin-based carrier such as a cyclodextrin polymer-nucleic acid complex.

In a further example, the carrier is a protein-based carrier such as a cationic peptide-nucleic acid complex.

In another example, the carrier is a lipid nanoparticle. Exemplary nanoparticles are described, for example, in U.S. Pat. No. 7,514,099.

In some examples, the nucleic acid(s), ddRNAi construct(s), or expression vector(s) of the disclosure is/are formulated with a lipid nanoparticle composition comprising a cationic lipid/Cholesterol/PEG-C-DMA/DSPC (e.g., in a 40/48/2/10 ratio), a cationic lipid/Cholesterol/PEG-DMG/DSPC (e.g., in a 40/48/2/10 ratio), or a cationic lipid/Cholesterol/PEG-DMG (e.g., in a 60/38/2 ratio). In some examples, the cationic lipid is Octyl CL in DMA, DL in DMA, L-278, DLinKC2DMA, or MC3.

In another example, the nucleic acid(s), ddRNAi construct(s), or expression vector(s) of the disclosure is/are formulated with any of the cationic lipid formulations described in WO 2010/021865; WO 2010/080724; WO 2010/042877; WO 2010/105209 or WO 2011/022460.

In another example, the nucleic acid(s) or ddRNAi construct(s), or expression vector(s) of the disclosure is/are conjugated to or complexed with another compound, e.g., to facilitate delivery of the nucleic acid(s), ddRNAi construct(s), or expression vector(s). Non-limiting, examples of such conjugates are described in US 2008/0152661 and US 2004/0162260 (e.g., CDM-LBA, CDM-Pip-LBA, CDM-PEG, CDM-NAG, etc.).

In another example, polyethylene glycol (PEG) is covalently attached to a nucleic acid or ddRNAi construct or DNA construct or expression vector of the disclosure. The attached PEG can be any molecular weight, e.g., from about 100 to about 50,000 daltons (Da).

In yet other example, the nucleic acid(s), ddRNAi construct(s), DNA construct, or expression vector(s) of the disclosure is/are formulated with a carrier comprising surface-modified liposomes containing poly(ethylene glycol) lipids (PEG-modified, or long-circulating liposomes or stealth liposomes), such as is disclosed in for example, WO 96/10391; WO 96/10390; or WO 96/10392.

In some examples, the nucleic acid(s), ddRNAi construct(s), DNA construct, or expression vector(s) of the disclosure can also be formulated or complexed with polyethyleneimine or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In other examples, the nucleic acid(s), ddRNAi construct(s), DNA construct, or expression vector(s) of the disclosure is/are complexed with membrane disruptive agents such as those described in U.S. Patent Application Publication No. 2001/0007666.

Other carriers include cyclodextrins (see for example, Gonzalez et al., 1999, Bioconjugate Chem., 10, 1068-1074; or WO 03/46185), poly(lactic-co-glycolic)acid (PLGA) and PLCA microspheres (see for example US 2002130430).

Compositions will desirably include materials that increase the biological stability of the nucleic acid(s), ddRNAi construct(s), DNA construct, or expression vector(s) of the disclosure and/or materials that increase the ability of the compositions to localise to and/or penetrate muscle cells selectively. The therapeutic compositions of the disclosure may be administered in pharmaceutically acceptable carriers (e.g., physiological saline), which are selected on the basis of the mode and route of administration, and standard pharmaceutical practice. One having ordinary skill in the art can readily formulate a pharmaceutical composition that comprises one or more nucleic acid(s), ddRNAi construct(s), DNA construct, or expression vector(s) of the disclosure. In some cases, an isotonic formulation is used. Generally, additives for isotonicity can include sodium chloride, dextrose, mannitol, sorbitol and lactose. In some cases, isotonic solutions such as phosphate buffered saline are preferred. Stabilizers include gelatin and albumin. In some examples, a vasoconstriction agent is added to the formulation. The compositions according to the present disclosure are provided sterile and pyrogen free. Suitable pharmaceutical carriers, as well as pharmaceutical necessities for use in pharmaceutical formulations, are described in Remington: The Science and Practice of Pharmacy (formerly Remington's Pharmaceutical Sciences), Mack Publishing Co., a standard reference text in this field, and in the USP/NF.

The volume, concentration, and formulation of the pharmaceutical composition, as well as the dosage regimen may be tailored specifically to maximize cellular delivery while minimizing toxicity such as an inflammatory response e.g, relatively large volumes (5, 10, 20, 50 ml or more) with corresponding low concentrations of active ingredients, as well as the inclusion of an anti-inflammatory compound such as a corticosteroid, may be utilized if desired.

Compositions of the disclosure may be formulated for administration by any suitable route (e.g., suitable for delivery to the pharyngeal muscle of a subject). For example, routes of administration include, but are not limited to, intramuscular, intraperitoneal, intradermal, subcutaneous, intravenous, intraarterially, intraoccularly and oral as well as transdermal or by inhalation or suppository. Exemplary routes of administration include intravenous (IV), intramuscular (IM), oral, intraperitoneal, intradermal, intraarterial and subcutaneous injection. In one example, the composition of the disclosure is formulated for IM administration (e.g., formulated for administration to the pharyngeal muscle). In a preferred embodiment, the administration is directly to the pharyngeal muscle of a subject. Such compositions are useful for pharmaceutical applications and may readily be formulated in a suitable sterile, non-pyrogenic vehicle, e.g., buffered saline for injection, for parenteral administration e.g., IM (e.g., directly to the pharyngeal muscle), intravenously (including intravenous infusion), SC, and for intraperitoneal administration. In a preferred embodiment, the route of administration, such as IM (e.g., directly to the pharyngeal muscle) achieves effective delivery to muscle tissue and transfection of a ddRNAi constructs and/or codon-optimised nucleic acids encoding PABPN1 of the disclosure, and expression of RNA and/or the codon-optimised nucleic acid therein.

TABLE 1
Targeted regions in PABPN1

| Region | Region sequence | |
|---|---|---|
| ID | (5′-3′) | SEQ ID NO: |
| Region 2 | GAGAAGCAGAUGAA | SEQ ID NO: 1 |
| | UAUGAGUCCACCUC | |
| Region 3 | GAACGAGGUAGAGA | SEQ ID NO: 2 |
| | AGCAGAUGAAUAUG | |
| Region 4 | GAAGCUGAGAAGCU | SEQ ID NO: 3 |
| | AAAGGAGCUACAGA | |
| Region 5 | GGGCUAGAGCGACA | SEQ ID NO: 4 |
| | UCAUGGUAUUCCCC | |
| Region 6 | CUGUGUGACAAAUU | SEQ ID NO: 5 |
| | UAGUGGCCAUCCCA | |
| Region 7 | GACUAUGGUGCAAC | SEQ ID NO: 6 |
| | AGCAGAAGAGCUGG | |
| Region 9 | CGAGGUAGAGAAGC | SEQ ID NO: 7 |
| | AGAUGAAUAUGAGU | |
| Region 11 | CAGUGGUUUUAACA | SEQ ID NO: 8 |
| | GCAGGCCCCGGGGU | |
| Region 13 | AGAGCGACAUCAUG | SEQ ID NO: 9 |
| | GUAUUCCCCUUACU | |
| Region 14 | GGUAGAGAAGCAGA | SEQ ID NO: 10 |
| | UGAAUAUGAGUCCA | |
| Region 15 | AUUGAGGAGAAGAU | SEQ ID NO: 11 |
| | GGAGGCUGAUGCCC | |
| Region 16 | GGAGGAAGAAGCUG | SEQ ID NO: 12 |
| | AGAAGCUAAAGGAG | |
| Region 17 | AACGAGGUAGAGAA | SEQ ID NO: 13 |
| | GCAGAUGAAUAUGA | |

TABLE 2
shmiR effector and effector
complement sequences

| | Effector | | | |
|---|---|---|---|---|
| | Complement | | Effector | |
| | Sequence | SEQ ID | Sequence | SEQ ID |
| ShmiR ID | (5′-3′) | NO: | (5′-3′) | NO: |
| shmiR2 | AGCAGAUGAA | SEQ ID | UGGACUCAUA | SEQ ID |
| | UAUGAGUCCA | NO: 14 | UUCAUCUGCU | NO 15 |
| | | | U | |
| shmiR3 | GAGGUAGAGA | SEQ ID | UUCAUCUGCL | SEQ ID |
| | AGCAGAUGAA | NO: 16 | UCUCUACCUC | NO: 17 |
| | | | G | |
| shmiR4 | CUGAGAAGCU | SEQ ID | UAGCUCCUUU | SEQ ID |
| | AAAGGAGCUA | NO: 18 | AGCUUCUCAG | NO: 19 |
| | | | C | |
| shmiR5 | UAGAGCGACA | SEQ ID | AAUACCAUGA | SEQ ID |
| | UCAUGGUAUU | NO: 20 | UGUCGCUCUA | NO: 21 |
| | | | G | |
| shmiR6 | GUGACAAAUU | SEQ ID | AUGGCCACUA | SEQ ID |
| | UAGUGGCCAU | NO: 22 | AAUUUGUCAC | NO: 23 |
| | | | A | |
| shmiR7 | AUGGUGCAAC | SEQ ID | CUCULCUGCU | SEQ ID |
| | AGCAGAAGAG | NO: 24 | GUUGCACCAU | NO: 25 |
| | | | A | |
| shmiR9 | GUAGAGAAGC | SEQ ID | AUAUUCAUCU | SEQ ID |
| | AGAUGAAUAU | NO: 26 | GCUUCUCUAC | NO: 27 |
| | | | C | |
| shmiR11 | GGUUIRJAAC | SEQ ID | CGGGGCCUGC | SEQ ID |
| | AGCAGGCCCC | NO: 28 | UGUUAAAACC | NO 29 |
| | G | | A | |
| shmiR13 | CGACAUCAUG | SEQ ID | AGGGGAAUAC | SEQ ID |
| | GUAUUCCCCU | NO: 30 | CAUGAUGUCG | NO: 31 |
| | | | C | |
| shmiR14 | GAGAAGCAGA | SEQ ID | CUCAUAUUCA | SEQ ID |
| | UGAAUAUGAG | NO: 32 | UCUGCUUCUC | NO: 33 |
| | | | U | |
| shmiRI5 | AGGAGAAGAU | SEQ ID | AUCAGCCUCC | SEQ ID |
| | GGAGGCUGAU | NO: 34 | AUCUUCUCCU | NO: 35 |
| | | | C | |
| shmiRI6 | GAAGAAGCUG | SEQ ID | UUUAGCUUCU | SEQ ID |
| | AGAAGCUAAA | NO: 36 | CAGCUUCUUC | NO: 37 |
| | | | C | |
| shmiR17 | AGGUAGAGAA | SEQ ID | AUUCAUCUGC | SEQ ID |
| | GCAGAUGAAU | NO: 38 | UUCUCUACCU | NO: 39 |
| | | | C | |

TABLE 3
shmiR sequences

| | shmiR sequences | |
|---|---|---|
| shmiR | (5′-3′) | SEQ ID NO: |
| shmiR2 | GGUAUAUUGCUGUUG | SEQ ID NO: 43 |
| | ACAGUGAGCGUAGCA | |
| | GAUGAAUAUGAGUCC | |
| | AACUGUGAAGCAGAU | |
| | GGGUUGGACUCAUAU | |
| | UCAUCUGCUUCGCCU | |
| | ACUGCCUCGGACUUC | |
| | AA | |
| shmiR3 | GGUAUAUUGCUGUUG | SEQ ID NO: 44 |
| | ACAGUGAGCGAGAGG | |
| | UAGAGAAGCAGAUGA | |
| | AACUGUGAAGCAGAU | |
| | GGGUUUCAUCUGCUU | |
| | CUCUACCUCGCGCCU | |
| | ACUGCCUCGGACUUC | |
| | AA | |
| shmiR4 | GGUAUAUUGCUGUUG | SEQ ID NO: 45 |
| | ACAGUGAGCGACUGA | |
| | GAAGCUAAAGGAGCU | |
| | AACUGUGAAGCAGAU | |
| | GGGUUAGCUCCUUUA | |
| | GCUUCUCAGCCGCCU | |
| | ACUGCCUCGGACUUC | |
| | AA | |
| shmiR5 | GGUAUAUUGCUGUUG | SEQ ID NO: 46 |
| | ACAGUGAGCGAUAGA | |
| | GCGACAUCAUGGUAU | |
| | UACUGUGAAGCAGAU | |
| | GGGUAAUACCAUGAU | |
| | GUCGCUCUAGCGCCU | |
| | ACUGCCUCGGACUUC | |
| | AA | |
| shmiR6 | GGUAUAUUGCUGUUG | SEQ ID NO: 47 |
| | ACAGUGAGCGAGUGA | |
| | CAAAUUUAGUGGCCA | |
| | UACUGUGAAGCAGAU | |
| | GGGUAUGGCCACUAA | |
| | AUUUGUCACACGCCU | |
| | ACUGCCUCGGACUUC | |
| | AA | |
| shmiR7 | GGUAUAUUGCUGUUG | SEQ ID NO: 48 |
| | ACAGUGAGCGAAUGG | |
| | UGCAACAGCAGAAGA | |
| | GACUGUGAAGCAGAU | |
| | GGGUCUCUUCUGCUG | |
| | UUGCACCAUACGCCU | |
| | ACUGCCUCGGACUUC | |
| | AA | |
| shmiR9 | GGUAUAUUGCUGUUG | SEQ ID NO: 49 |
| | ACAGUGAGCGAGUAG | |
| | AGAAGCAGAUGAAUA | |
| | UACUGUGAAGCAGAU | |
| | GGGUAUAUUCAUCUG | |
| | CUUCUCUACCCGCCU | |
| | ACUGCCUCGGACUUC | |
| | AA | |
| shmiR11 | GGUAUAUUGCUGUUG | SEQ ID NO: 50 |
| | ACAGUGAGCGAGGUU | |
| | UUAACAGCAGGCCCC | |
| | GACUGUGAAGCAGAU | |
| | GGGUCGGGGCCUGCU | |
| | GUUAAAACCACGCCU | |
| | ACUGCCUCGGACUUC | |
| | AA | |
| shmiR13 | GGUAUAUUGCUGUUG | SEQ ID NO: 51 |
| | ACAGUGAGCGACGAC | |
| | AUCAUGGUAUUCCCC | |
| | UACUGUGAAGCAGAU | |
| | GGGUAGGGGAAUACC | |
| | AUGAUGUCGCCGCCU | |
| | ACUGCCUCGGACUUC | |
| | AA | |
| shmiR14 | GGLAUAUUGCUGUUG | SEQ ID NO: 52 |
| | ACAGUGAGCGUGAGA | |
| | AGCAGAUGAAUAUGA | |
| | GACUGUGAAGCAGAU | |
| | GGGUCUCAUAUUCAU | |
| | CUGCUUCUCUCGCCU | |
| | ACUGCCUCGGACUUC | |
| | AA | |
| shmiR15 | GGUAUAUUGCUGUUG | SEQ ID NO: 53 |
| | ACAGUGAGCGAAGGA | |
| | GAAGAUGGAGGCUGA | |
| | UACUGUGAAGCAGAU | |
| | GGGUAUCAGCCUCCA | |
| | UCUUCUCCUCCGCCU | |
| | ACUGCCUCGGACUUC | |
| | AA | |
| shmiR16 | GGUAUAUUGCUGUUG | SEQ ID NO: 54 |
| | ACAGUGAGCGAGAAG | |
| | AAGCUGAGAAGCUAA | |
| | AACUGUGAAGCAGAU | |
| | GGGUUUUAGCUUCUC | |
| | AGCUUCUUCCCGCCU | |
| | ACUGCCUCGGACUUC | |
| | AA | |
| shmiR17 | GGUAUAUUGCUGUUG | SEQ ID NO: 55 |
| | ACAGUGAGCGAAGGU | |
| | AGAGAAGCAGAUGAA | |
| | UACUGUGAAGCAGAU | |
| | GGGUAUUCAUCUGCU | |
| | UCUCUACCUCCGCCU | |
| | ACUGCCUCGGACUUC | |
| | AA | |

Shmir encoding cassettes

| | Shmir encoding | |
|---|---|---|
| | Cassettes | |
| shmiR | (5′-3′) | SEQ ID NO: |
| shmiR2 | GGTATATTGCTGTTG | SEQ ID NO: 56 |
| | ACAGTGAGCGTAGCA | |
| | GATGAATATGAGTCC | |
| | AACTGTGAAGCAGAT | |
| | GGGTTGGACTCATAT | |
| | TCATCTGCTTCGCCT | |
| | ACTGCCTCGGACTTC | |
| | AA | |
| shmiR3 | GGTATATTGCTGTTG | SEQ ID NO: 57 |
| | ACAGTGAGCGAGAGG | |
| | TAGAGAAGCAGATGA | |
| | AACTGTGAAGCAGAT | |
| | GGGTTTCATCTGCTT | |
| | CTCTACCTCGCGCCT | |
| | ACTGCCTCGGACTTC | |
| | AA | |
| shmiR4 | GGTATATTGCTGTTG | SEQ ID NO: 58 |
| | ACAGTGAGCGACTGA | |
| | GAAGCTAAAGGAGCT | |
| | AACTGTGAAGCAGAT | |
| | GGGTTAGCTCCTTTA | |
| | GCTTCTCAGCCGCCT | |
| | ACTGCCTCGGACTTC | |
| | AA | |
| shmiR5 | GGTATATTGCTGTTG | SEQ ID NO: 59 |
| | ACAGTGAGCGATAGA | |
| | GCGACATCATGGTAT | |
| | TACTGTGAAGCAGAT | |
| | GGGTAATACCATGAT | |
| | GTCGCTCTAGCGCCT | |
| | ACTGCCTCGGACTTC | |
| | AA | |
| shmiR6 | GGTATATTGCTGTTG | SEQ ID NO: 60 |
| | ACAGTGAGCGAGTGA | |
| | CAAATTTAGTGGCCA | |
| | TACTGTGAAGCAGAT | |
| | GGGTATGGCCACTAA | |
| | ATTTGTCACACGCCT | |
| | ACTGCCTCGGACTTC | |
| | AA | |
| shmiR7 | GGTATATTGCTGTTG | SEQ ID NO: 61 |
| | ACAGTGAGCGAATGG | |
| | TGCAACAGCAGAAGA | |
| | GACTGTGAAGCAGAT | |
| | GGGTCTCTTCTGCTG | |
| | TTGCACCATACGCCT | |
| | ACTGCCTCGGACTTC | |
| | AA | |
| shmiR9 | GGTATATTGCTGTTG | SEQ ID NO: 62 |
| | ACAGTGAGCGAGTAG | |
| | AGAAGCAGATGAATA | |
| | TACTGTGAAGCAGAT | |
| | GGGTATATTCATCTG | |
| | CTTCTCTACCCGCCT | |
| | ACTGCCTCGGACTTC | |
| | AA | |
| shmiR11 | GGTATATTGOTGTTG | SEQ ID NO: 63 |
| | ACAGTGAGCGAGGTT | |
| | TTAACAGOAGGCCCC | |
| | GACTGTGAAGCAGAT | |
| | GGGTCGGGGCCTGCT | |
| | GTTAAAACCACGCCT | |
| | ACTGCCTCGGACTTC | |
| | AA | |
| shmiR13 | GGTATATTGCTGTTG | SEQ ID NO: 64 |
| | ACAGTGAGCGACGAC | |
| | ATCATGGTATTCCCC | |
| | TACTGTGAAGCAGAT | |
| | GGGTAGGGGAATACC | |
| | ATGATGTCGCCGCCT | |
| | ACTGCCTCGGACTTC | |
| | AA | |
| shmiR14 | GGTATATTGCTGTTG | SEQ ID NO: 65 |
| | ACAGTGAGCGTGAGA | |
| | AGCAGATGAATATGA | |
| | GACTGTGAAGCAGAT | |
| | GGGTCTCATATTCAT | |
| | CTGCTTCTCTCGCCT | |
| | ACTGCCTCGGACTTC | |
| | AA | |
| shmiR15 | GGTATATTGCTGTTG | SEQ ID NO: 66 |
| | ACAGTGAGCGAAGGA | |
| | GAAGATGGAGGCTGA | |
| | TACTGTGAAGCAGAT | |
| | GGGTATCAGCCTCCA | |
| | TCTTCTCCTCCGCCT | |
| | ACTGCCTCGGACTTC | |
| | AA | |
| shmiR16 | GGTATATTGCTGTTG | SEQ ID NO: 67 |
| | ACAGTGAGCGAGAAG | |
| | AAGCTGAGAAGCTAA | |
| | AACTGTGAAGCAGAT | |
| | GGGTTTTAGCTTCTC | |
| | AGCTTCTTCCCGCCT | |
| | ACTGCCTCGGACTTC | |
| | AA | |
| shmiR17 | GGTATATTGCTGTTG | SEQ ID NO: 68 |
| | ACAGTGAGCGAAGGT | |
| | AGAGAAGCAGATGAA | |
| | TACTGTGAAGCAGAT | |
| | GGGTATTCATCTGCT | |
| | TCTCTACCTCCGCCT | |
| | ACTGCCTCGGACTTC | |
| | AA | |

### Example 1—Design of shmiRs Targeting PABPN1

Sequences representing potential targets for design of siRNA constructs were identified from the PABPN1 mRNA sequence using publicly available siRNA design algorithms (including Ambion, Promega, Invitrogen, Origene and MWG): the selected sequences were conserved in humans, non-human primates, bovine and mice species. Sequences encoding the candidate siRNAs were incorporated into a pre-miR30a scaffold in order to create a sequence encoding a short-hairpin microRNA (shmiR) comprising a 5′ flanking region (SEQ ID NO: 41), a siRNA sense strand sequence (effector complement sequence), a stem/loop junction sequence (SEQ ID NO: 40), a siRNA anti-sense strand (effector sequence), and a 3′ flanking region (SEQ ID NO:42). The predicted secondary structure of a representative shmiR is shown in FIG. 1C. The target regions of the PABPN1 mRNA transcript for the designed shmiRs are presented in Table 1 and corresponding shmiR effector sequences (antisense strand) are presented in Table 2.

### Example 2—Generation of a Single “Silence and Replace Construct” for Simultaneous Gene Silencing of Endogenous PABPN1 and Replacement with Codon Optimised PABPN1

A single stranded adeno-associated virus type 2 (ssAAV2) plasmid expressing shmiR17 and shmiR13 (e.g., as described in Tables 3 and 4) in combination with the optPABPN1 sequence was created.

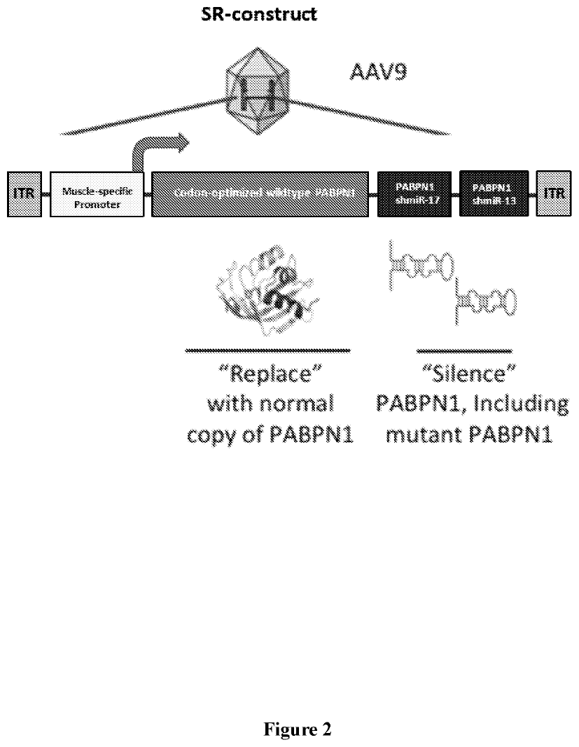

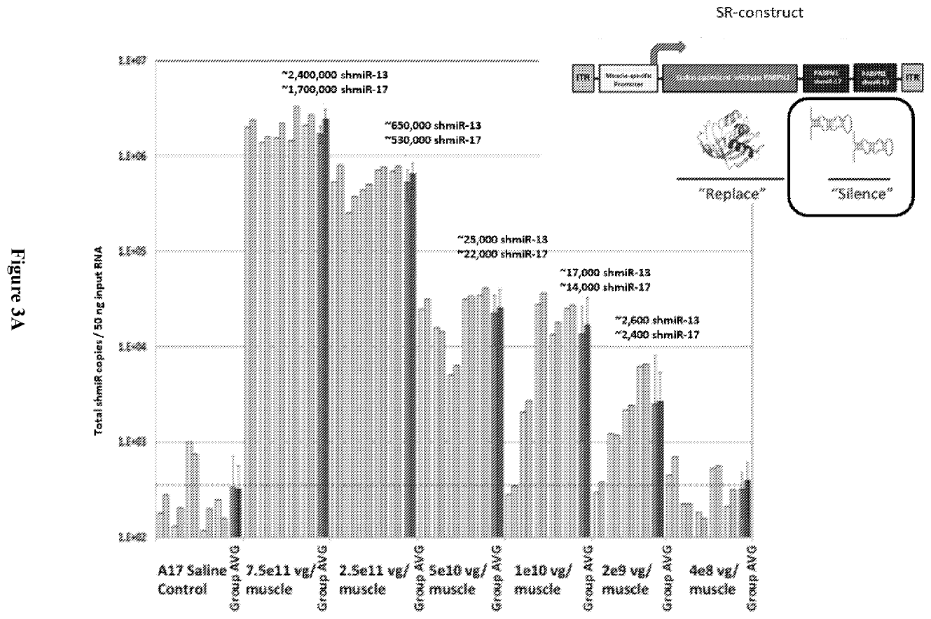

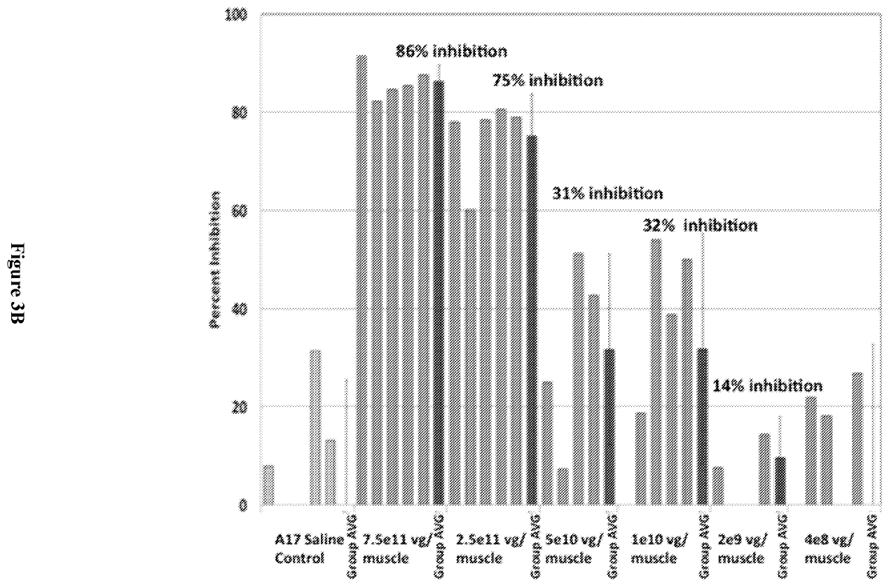

The silence and replace construct (hereinafter “SR-construct”) was generated by subcloning DNA sequences encoding shmiR17 and shmiR13 (as described in Table 4) into the 3′ untranslated region of the optPABPN1 transcript in the pAAV2 vector backbone (pAAV-shmiR viral plasmid). Expression of both optPABPN1 and the two shmiRs in a single transcript is driven by the muscle specific promoter Spc512. A schematic of the SR-construct is provided in FIG. 1(A), FIG. 1(B), and FIG. 2.

Recombinant pseudotyped AAV vector stocks were then generated. Briefly, HEK293T cells were cultured in cell factories in Dulbecco's modified Eagle's medium, supplemented with 10% FBS, and incubated at 37° C. and 5% CO₂. The pAAV-shmiR viral plasmid (the SR-construct) and a pAAVhelper and pAAVrepcap8 plasmid or pAAVhelper and pAAV repcap9 or pAAV helper and pAAVRH74 plasmid (as described in WO2013123503A1) were complexed with Calcium Phosphate according to the manufacturer's instructions. Triple-transfections were then performed with the pAAV-shmiR plasmid (the SR-construct) in combination with the pAAVhelper and one of the following capsids; pAAVrepcap8, pAAVrepcap9 or pAAVRH74, in the HEK293T cells. The HEK293T cells were then cultured for a period of 72 hours at 37° C. and 5% CO₂, after which time the cells were lysed and particles expressing the SR-construct were purified by iodixanol (Sigma-Aldrich) step-gradient ultracentrifugation followed by cesium chloride ultracentrifugation. The number of vector genomes was quantified by quantitative polymerase chain reaction (Q-PCR).

### Example 3—In Vivo Studies with a Single Vector “Silence and Replace” Approach

In order to test the in vivo efficacy of the SR-construct described in Example 2 in a relevant disease model of OPMD, the SR-construct was administered individually, at a range of doses, via intramuscular injection into the Tibialis anterior (TA) muscle of 10-12 week old A17 mice. The doses were set at 7.5×10¹¹, 2.5×10¹¹, 5×10¹⁹, 1×10¹⁹, 2×10⁹, and 4×10⁸ vector genomes (vg) per muscle. Saline injected age-matched A17 mice served as the untreated group. Mice were sacrificed at either 14 or 20 weeks post treatment.

### Example 4—Quantitative Measurements of shmiR Production, PABPN1 Silencing, and Codon-Optimized PABPN1 Expression in SR-Construct Treated A17 Mice

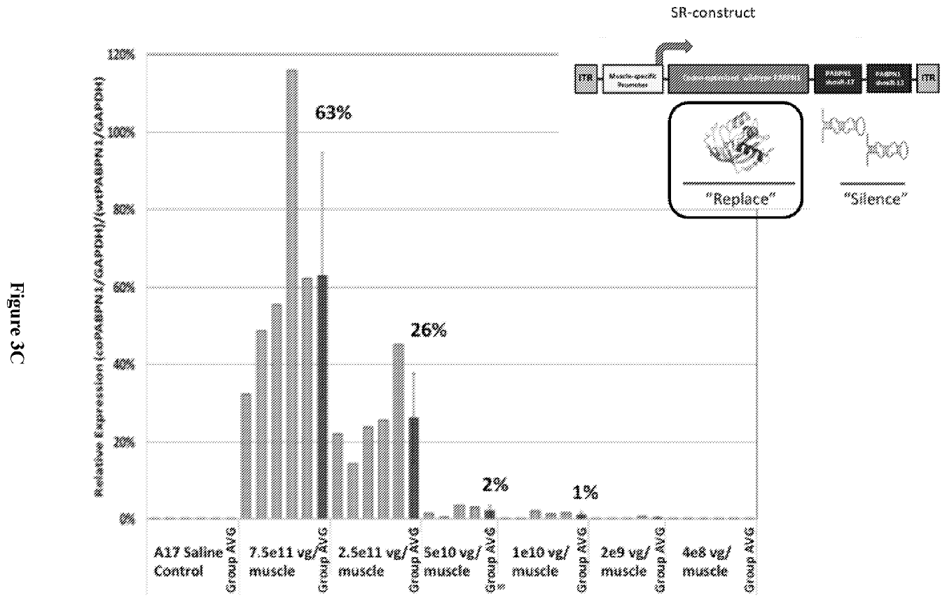

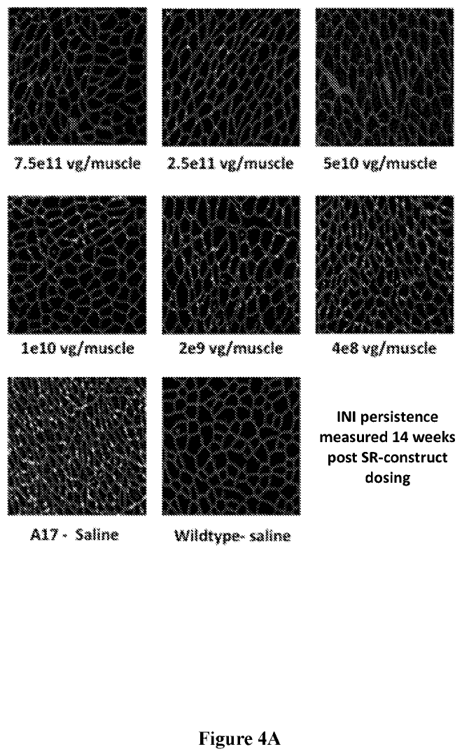

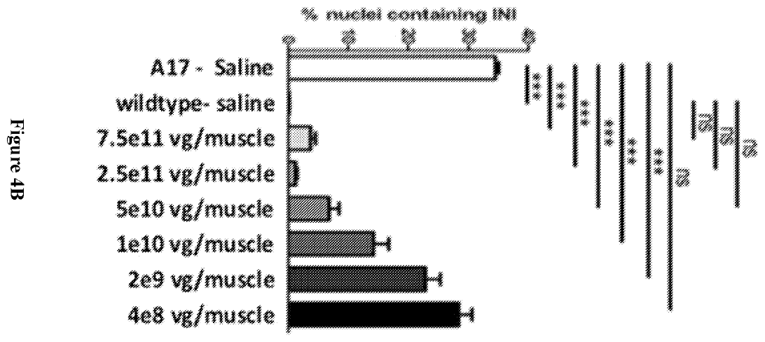

Fourteen weeks after SR-construct treatment, the TA muscles of the A17 mice of Example 3 were harvested and RNA extracted. SR-construct-dependent expression of shmiRs in TA muscles was quantified (FIG. 3A). The quantified expression level of shmiRs was dependent on SR-construct dose, as was silencing of PABPN1 (including expPABPN1) (FIG. 3B), and restoration of normal PABPN1 levels (FIG. 3C).

### Example 5—Reduction of Intranuclear Inclusions (INIs) in SR-Construct Treated A17 Mice

The impact of the SR-construct on the persistence of intranuclear inclusions (INIs) was tested in the week 14 A17 mice of Example 3. FvB wildtype mice were also included as healthy comparators. Fourteen weeks after AAV injection, muscles were collected and mounted for histological studies. Sections were pre-treated with 1M KCl to preferentially elute all soluble PABPN1 from the tissue. Immunofluorescence for PABPN1 (green) and Laminin, an abundant protein in the extracellular matrix of muscle cells (red) was detected in sections of treated muscles and showed significant reduction in the number of PABPN1-positive intranuclear inclusions (INIs) in SR-construct-treated muscles with a dose effect (FIG. 4A). Quantification of percentage of nuclei containing INIs in muscle sections indicates that treatment with the SR-construct significantly reduces the amount of INIs compared to untreated A17 muscles (One-way Anova test with Bonferroni post-doc test, ***p<0.001, ns: not significant) (FIG. 4B).

### Example 6—Treatment with the SR-Construct Improves the Physiological Properties and Functionality of Treated Muscles

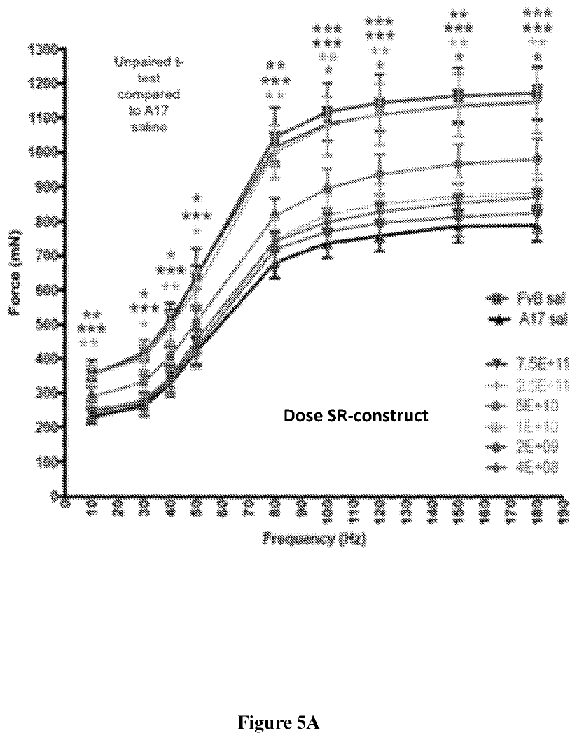

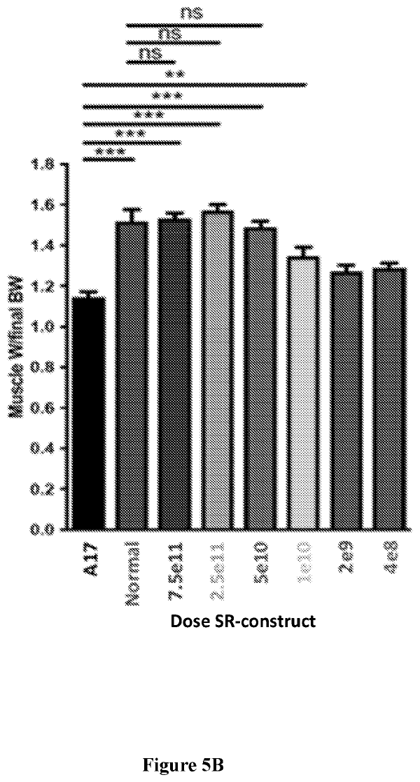

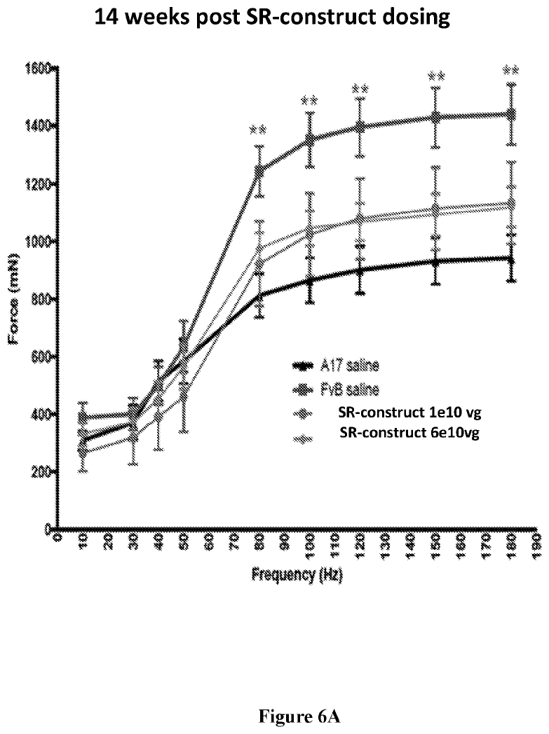

Physiological properties and functionality of treated muscles were measured in the week 14 A17 mice of Example 3. FvB wildtype mice were also included as healthy comparators. Maximal force generated by TA muscles was measured by in situ muscle physiology (FIG. 5A). SR-construct significantly increased the maximal force generated by TA muscles in a dose-dependent manner. Muscle weight normalized to body weight (BW) was also measured 14 weeks post SR-construct dosing (FIG. 5B). Muscle weight normalized to body weight of SR-treated muscles was comparable to that of control FvB mice at doses above 1e10 vg per TA injected (mean±SEM n=10, One-way Anova test with Bonferroni post-doc test, *p<0.05, ***p<0.001, **p<0.01, ns: not significant).

### Example 7—Restoration of Muscle Function Over Time

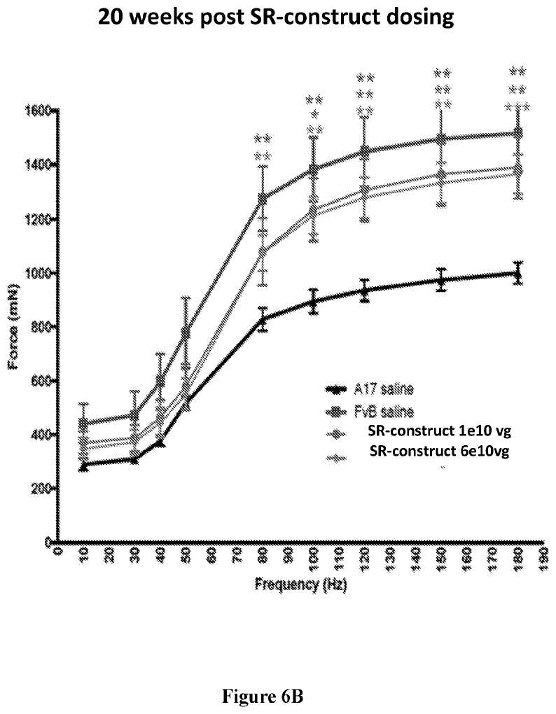

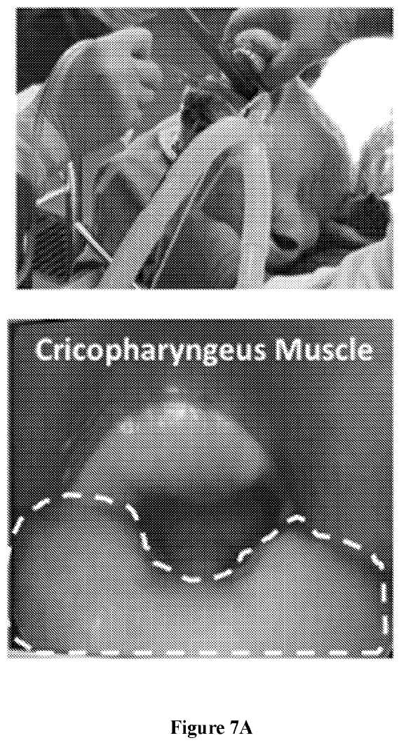

Maximal force generated by TA muscles of SR-construct-treated A17 mice and FvB wildtype mice was measured by in situ muscle physiology at 14 weeks post SR-construct dosing (FIG. 6A) and at 20 weeks post SR-construct dosing (FIG. 6B). For intermediate doses (1e10 vg and 6e10 vg per TA), beneficial effect on muscle force was much more pronounced at 20 weeks compared to 14 weeks after injection (mean±SEM n=10, One-way Anova test with Bonferroni post-doc test, ***p<0.001, **p<0.01).

### Example 8—Direct Administration to Pharyngeal Muscle of Sheep

Direct injection of the SR-construct to the pharyngeal muscles of sheep was tested PABPN1 is highly conserved from sheep to humans including all but one amino acid residue at position 95.

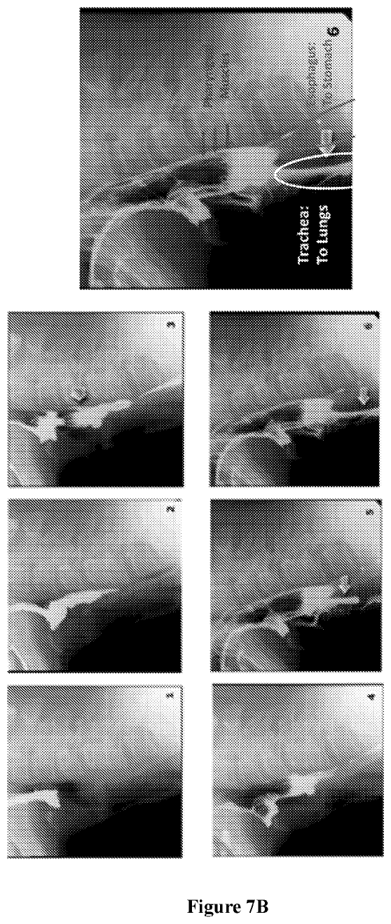

The SR-construct was directly injected into pharyngeal muscles of sheep (FIG. 7A). Two animals in the sheep study were each injected with 1.5e13 vg SR-construct into the cricopharyngeus muscle (CP) and an additional 1.0e13 vg SR-construct into the pharyngeal muscles (pharynx). The remaining 10 animals treated with SR-construct (1.0e10 vg to 1.0e13 vg) only received injections into the CP. The CP was injected with a total volume of 1.5 ml (3 injections of 0.5 ml each). The pharynx was injected with a total volume of 6 ml (2 injections of 1.5 ml on both the right and left sides).

Radioimaging using a radiolabeled cream illustrates the severe dysphagia in human OPMD patients with risks of “fausse route” (FIG. 7B).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for treating a subject suffering from oculopharyngeal muscular dystrophy (OPMD) comprising administering to said subject a composition comprising:
(a) a ddRNAi construct comprising a nucleic acid comprising a DNA sequence which encodes a short hairpin micro-RNA (shmiR); and
(b) a PABPN1 construct comprising a DNA sequence encoding a functional PABPN1 protein having a mRNA transcript which is not targeted by the shmiR(s) encoded by the nucleic acid; wherein the composition is administered by direct injection to a pharyngeal muscle of the subject.

2. (canceled)

3. The method of claim 1, wherein the subject has improved swallowing following administering the composition by direct injection to a pharyngeal muscle of the subject.

4. The method of claim 1, wherein the composition comprises an expression vector comprising the ddRNAi construct, the PABPN1 construct, or a combination thereof.

5. The method of claim 4, wherein the expression vector comprises, in a 5′ to 3′ direction, the ddRNAi construct and the PABPN1 construct.

6. The method of claim 4, wherein the expression vector comprises, in a 5′ to 3′ direction, the PABPN1 construct and the ddRNAi construct.

7. The method of claim 4, wherein the expression vector is a plasmid or minicircle.

8. The method of claim 4, wherein the expression vector is a viral vector selected from the group consisting of an adeno-associated viral (AAV) vector, a retroviral vector, an adenoviral (AdV) vector and a lentiviral (LV) vector.

9. The method of claim 4, wherein the ddRNAi construct and/or PABPN1 construct is/are comprised within an expression construct and the expression construct comprises inverted terminal repeats (ITRs) from an AAV serotype, optionally wherein the AAV serotype is AAV2, AAV8 or AAV9.

10. (canceled)

11. The method of claim 1, wherein the DNA sequence encoding the functional PABPN1 protein is codon optimised such that its mRNA transcript is not targeted by the shmiRs of the ddRNAi construct, optionally wherein the DNA sequence encoding the functional PABPN1 protein is set forth in SEQ ID NO: 73.

12. (canceled)

13. The method of claim 1, wherein the DNA sequence encoding the functional PABPN1 protein is operably-linked to a promoter comprised within the PABPN1 construct and positioned upstream of the DNA sequence encoding the functional PABPN1 protein, optionally wherein the promoter comprised within the PABPN1 construct is a muscle-specific promoter.

14. (canceled)

15. The method of claim 1, wherein the shmiR comprises:
an effector sequence of at least 17 nucleotides in length;
an effector complement sequence;
a stemloop sequence; and
a primary micro RNA (pri-miRNA) backbone;
wherein the effector sequence is substantially complementary to a region of corresponding length in an RNA transcript of human PABPN1.

16. The method of claim 15, wherein:
(i) the shmiR comprises an effector sequence which is substantially complementary to a region of corresponding length within the RNA sequence set forth in SEQ ID NO: 87;
(ii) the shmiR comprises an effector sequence which is substantially complementary to a region of corresponding length in an RNA transcript set forth in any one of SEQ ID NOs: 1-13; and/or
(iii) the shmiR is selected from the group consisting of:
a shmiR comprising an effector sequence set forth in SEQ ID NO: 15 and an effector complement sequence set forth in SEQ ID NO: 14;
a shmiR comprising an effector sequence set forth in SEQ ID NO: 17 and an effector complement sequence set forth in SEQ ID NO: 16;
a shmiR comprising an effector sequence set forth in SEQ ID NO: 19 and an effector complement sequence set forth in SEQ ID NO: 18;
a shmiR comprising an effector sequence set forth in SEQ ID NO: 21 and an effector complement sequence set forth in SEQ ID NO: 20;
a shmiR comprising an effector sequence set forth in SEQ ID NO: 23 and an effector complement sequence set forth in SEQ ID NO: 22;
a shmiR comprising an effector sequence set forth in SEQ ID NO: 25 and an effector complement sequence set forth in SEQ ID NO: 24;
a shmiR comprising an effector sequence set forth in SEQ ID NO: 27 and an effector complement sequence set forth in SEQ ID NO: 26;
a shmiR comprising an effector sequence set forth in SEQ ID NO: 29 and an effector complement sequence set forth in SEQ ID NO: 28;
a shmiR comprising an effector sequence set forth in SEQ ID NO: 31 and an effector complement sequence set forth in SEQ ID NO: 30;
a shmiR comprising an effector sequence set forth in SEQ ID NO: 33 and an effector complement sequence set forth in SEQ ID NO: 32;
a shmiR comprising an effector sequence set forth in SEQ ID NO: 35 and an effector complement sequence set forth in SEQ ID NO: 34;
a shmiR comprising an effector sequence set forth in SEQ ID NO: 37 and an effector complement sequence set forth in SEQ ID NO: 36; and
a shmiR comprising an effector sequence set forth in SEQ ID NO: 39 and an effector complement sequence set forth in SEQ ID NO: 38.

17. (canceled)

18. (canceled)

19. The method of claim 1, wherein the shmiR comprises, in a 5′ to 3′ direction:
a 5′ flanking sequence of the pri-miRNA backbone;
the effector complement sequence;
the stemloop sequence;
the effector sequence; and
a 3′ flanking sequence of the pri-miRNA backbone;
optionally wherein:
the stemloop sequence is the sequence set forth in SEQ ID NO: 40;
the pri-miRNA backbone is a pri-miR-30a backbone; and/or
the 5′ flanking sequence of the pri-miRNA backbone is set forth in SEQ ID NO: 41 and
the 3′ flanking sequence of the pri-miRNA backbone is set forth in SEQ ID NO: 42.

20.-22. (canceled)

23. The method of claim 1, wherein:
the shmiR comprises a sequence set forth in any one of SEQ ID NOs: 43-55; and/or
the DNA sequence which encodes the shmiR is set forth in any one of SEQ ID NO: 56-68.

24. (canceled)

25. The method of claim 1, comprising administering at least two nucleic acids encoding shmiRs, or administering a ddRNAi construct comprising the at least two nucleic acids, wherein each shmiR comprises an effector sequence which is substantially complementary to a RNA transcript corresponding to a PABPN1 protein which is causative of OPMD, and wherein each shmiR comprises a different effector sequence.

26. The method of claim 25, wherein:
(i) each of the at least two nucleic acids encode a shmiR comprising an effector sequence which is substantially complementary to a region of corresponding length in an RNA transcript set forth in one of SEQ ID NOs: 1, 2, 4, 7, 9, 10 and 13;
(ii) the at least two nucleic acids are selected from the group consisting of:
a nucleic acid comprising or consisting of a DNA sequence encoding a shmiR comprising an effector sequence set forth in SEQ ID NO: 15 and an effector complement sequence set forth in SEQ ID NO: 14 (shmiR2);
a nucleic acid comprising or consisting of a DNA sequence encoding a shmiR comprising an effector sequence set forth in SEQ ID NO: 17 and an effector complement sequence set forth in SEQ ID NO: 16 (shmiR3);
a nucleic acid comprising or consisting of a DNA sequence encoding a shmiR comprising an effector sequence set forth in SEQ ID NO: 21 and an effector complement sequence set forth in SEQ ID NO: 20 (shmiR5);
a nucleic acid comprising or consisting of a DNA sequence encoding a shmiR comprising an effector sequence set forth in SEQ ID NO: 27 and an effector complement sequence set forth in SEQ ID NO: 26 (shmiR9);
a nucleic acid comprising or consisting of a DNA sequence encoding a shmiR comprising an effector sequence set forth in SEQ ID NO: 31 and an effector complement sequence set forth in SEQ ID NO: 30 (shmiR13);
a nucleic acid comprising or consisting of a DNA sequence encoding a shmiR comprising an effector sequence set forth in SEQ ID NO: 33 and an effector complement sequence set forth in SEQ ID NO: 32 (shmiR14); and
a nucleic acid comprising or consisting of a DNA sequence encoding a shmiR comprising an effector sequence set forth in SEQ ID NO: 39 and an effector complement sequence set forth in SEQ ID NO: 38 (shmiR17); and/or
(iii) the at least two nucleic acids are selected from the group consisting of:
a nucleic acid comprising or consisting of a DNA sequence set forth in SEQ ID NO: 56 (shmiR2);
a nucleic acid comprising or consisting of a DNA sequence set forth in SEQ ID NO: 57 (shmiR3);
a nucleic acid comprising or consisting of a DNA sequence set forth in SEQ ID NO: 59 (shmiR5);
a nucleic acid comprising or consisting of a DNA sequence set forth in SEQ ID NO: 62 (shmiR9);
a nucleic acid comprising or consisting of a DNA sequence set forth in SEQ ID NO: 64 (shmiR13);
a nucleic acid comprising or consisting of a DNA sequence set forth in SEQ ID NO: 65 (shmiR14); and
a nucleic acid comprising or consisting of a DNA sequence set forth in SEQ ID NO: 68 (shmiR17).

27. (canceled)

28. (canceled)

29. The method of claim 25, wherein:
(i) each of the at least two nucleic acids encode a shmiR comprising an effector sequence which is substantially complementary to a region of corresponding length in an RNA transcript set forth in one of SEQ ID NOs: 2, 9, 10 and 13;
(ii) the at least two nucleic acids are selected from the group consisting of:
a nucleic acid comprising or consisting of a DNA sequence encoding a shmiR comprising an effector sequence set forth in SEQ ID NO: 17 and an effector complement sequence set forth in SEQ ID NO: 16 (shmiR3);
a nucleic acid comprising or consisting of a DNA sequence encoding a shmiR comprising an effector sequence set forth in SEQ ID NO: 31 and an effector complement sequence set forth in SEQ ID NO: 30 (shmiR13);
a nucleic acid comprising or consisting of a DNA sequence encoding a shmiR comprising an effector sequence set forth in SEQ ID NO: 33 and an effector complement sequence set forth in SEQ ID NO: 32 (shmiR14); and
a nucleic acid comprising or consisting of a DNA sequence encoding a shmiR comprising an effector sequence set forth in SEQ ID NO: 39 and an effector complement sequence set forth in SEQ ID NO: 38 (shmiR17); and/or
(iii) the at least two nucleic acids are selected from the group consisting of:
a nucleic acid comprising or consisting of a DNA sequence set forth in SEQ ID NO: 57 (shmiR3);
a nucleic acid comprising or consisting of a DNA sequence set forth in SEQ ID NO: 64 (shmiR13);
a nucleic acid comprising or consisting of a DNA sequence set forth in SEQ ID NO: 65 (shmiR14); and
a nucleic acid comprising or consisting of a DNA sequence set forth in SEQ ID NO: 68 (shmiR17).

30. (canceled)

31. (canceled)

32. The method of claim 1, wherein said ddRNAi construct comprises:
(i) a nucleic acid comprising or consisting of a DNA sequence encoding a shmiR comprising an effector sequence set forth in SEQ ID NO: 31 and an effector complement sequence set forth in SEQ ID NO: 30 (shmiR13); and
a nucleic acid comprising or consisting of a DNA sequence encoding a shmiR comprising an effector sequence set forth in SEQ ID NO: 39 and an effector complement sequence set forth in SEQ ID NO: 38 (shmiR17); and/or
(ii) a nucleic acid comprising or consisting of the DNA sequence set forth in SEQ ID NO: 64 (shmiR13); and
a nucleic acid comprising or consisting of the DNA sequence set forth in SEQ ID NO: 68 (shmiR17).

33. (canceled)

34. The method of claim 1, wherein the composition further comprises one or more pharmaceutically acceptable carriers.

35. The method of claim 1, wherein the pharyngeal muscle comprises one or more of an inferior constrictor muscle, a middle constrictor muscle, a superior constrictor muscle, a palatopharyngeus muscle, a salpingopharyngeus muscle, a stylopharyngeus muscle, or any combination thereof.
